SEQUENCE LISTING (58 sequences)

SEQ ID NO: 1 - DNA, 21 bp, Artificial Sequence
  A synthetic sequence
gggttgggtaaaggggtttta

SEQ ID NO: 2 - DNA, 29 bp, Artificial Sequence
  A synthetic sequence
ccatctaatctaaaatttacaacaaaaca

SEQ ID NO: 3 - DNA, 25 bp, Artificial Sequence
  A synthetic sequence
tttgttgtttaggttggtttgaagt

SEQ ID NO: 4 - DNA, 25 bp, Artificial Sequence
  A synthetic sequence
caaaaatataaccaatatcccaacc

SEQ ID NO: 5 - DNA, 26 bp, Artificial Sequence
  A synthetic sequence
tttggataattttttagaagttgaga

SEQ ID NO: 6 - DNA, 24 bp, Artificial Sequence
  A synthetic sequence
caaaaacatcaaaaacaaaaccac

SEQ ID NO: 7 - DNA, 25 bp, Artificial Sequence
  A synthetic sequence
aagggttatttggatttaggagttg

SEQ ID NO: 8 - DNA, 25 bp, Artificial Sequence
  A synthetic sequence
aacctaaccaatatcacccaaaata

SEQ ID NO: 9 - DNA, 25 bp, Artificial Sequence
  A synthetic sequence
ggggataatggagttaatatttagg

SEQ ID NO: 10 - DNA, 30 bp, Artificial Sequence
  A synthetic sequence
ccaaatttaaacaactactacaacactacc

SEQ ID NO: 11 - DNA, 25 bp, Artificial Sequence
  A synthetic sequence
ggtttgattggtttttttattaggg

SEQ ID NO: 12 - DNA, 24 bp, Artificial Sequence
  A synthetic sequence
acccctacctacctctttaactcc

SEQ ID NO: 13 - DNA, 24 bp, Artificial Sequence
  A synthetic sequence
ggattttggtgttaggtagggatt

SEQ ID NO: 14 - DNA, 25 bp, Artificial Sequence
  A synthetic sequence
atattacacaaaaaccccaaaaaaa

SEQ ID NO: 15 - DNA, 25 bp, Artificial Sequence
  A synthetic sequence
taataggggaagtaaggttttttgt

SEQ ID NO: 16 - DNA, 25 bp, Artificial Sequence
  A synthetic sequence
ccaaaactaaatcctaaaacccaaa

SEQ ID NO: 17 - DNA, 27 bp, Artificial Sequence
  A synthetic sequence
ggtgttgtgttttgtatggtattttat

SEQ ID NO: 18 - DNA, 24 bp, Artificial Sequence
  A synthetic sequence
taccccaaccaaatattcaaaaac

SEQ ID NO: 19 - DNA, 25 bp, Artificial Sequence
  A synthetic sequence
ggtttttatgaggtgtttaaattgg

SEQ ID NO: 20 - DNA, 22 bp, Artificial Sequence
  A synthetic sequence
taaactctcccacccaaacaaa

SEQ ID NO: 21 - DNA, 25 bp, Artificial Sequence
  A synthetic sequence
gtaagtttaagtttggtgttggggt

SEQ ID NO: 22 - DNA, 26 bp, Artificial Sequence
  A synthetic sequence
ccctctctctaaaaatttacattcca

SEQ ID NO: 23 - DNA, 26 bp, Artificial Sequence
  A synthetic sequence
ttgtagaagttgttgaaggaggtttt

SEQ ID NO: 24 - DNA, 27 bp, Artificial Sequence
  A synthetic sequence
aaaatctaaccaaactacaatcctacc

SEQ ID NO: 25 - DNA, 24 bp, Artificial Sequence
  A synthetic sequence
gtttatttttgttaggggtggttg

SEQ ID NO: 26 - DNA, 24 bp, Artificial Sequence
  A synthetic sequence
tatatcaaaaaatccccaaaaact

SEQ ID NO: 27 - DNA, 24 bp, Artificial Sequence
  A synthetic sequence
ggtagtgtaatttgtataaagagg

SEQ ID NO: 28 - DNA, 25 bp, Artificial Sequence
  A synthetic sequence
cacctcattctaaaattcccatatc

SEQ ID NO: 29 - DNA, 26 bp, Artificial Sequence
  A synthetic sequence
tgtttttaatagatagaaattgggtg

SEQ ID NO: 30 - DNA, 30 bp, Artificial Sequence
  A synthetic sequence
accaaaatataaaaataacatatattaccc

SEQ ID NO: 31 - DNA, 22 bp, Artificial Sequence
  A synthetic sequence
tttggtattttgggtagttggg

SEQ ID NO: 32 - DNA, 25 bp, Artificial Sequence
  A synthetic sequence
tatcaaaaacaaacaacaacaatcc

SEQ ID NO: 33 - DNA, 26 bp, Artificial Sequence
  A synthetic sequence
ggtaatttttggtagtgatttggatt

SEQ ID NO: 34 - DNA, 25 bp, Artificial Sequence
  A synthetic sequence
aaaacttattcctcttttcccctct

SEQ ID NO: 35 - DNA, 25 bp, Artificial Sequence
  A synthetic sequence
ataagtttttagataggggagtggg

SEQ ID NO: 36 - DNA, 25 bp, Artificial Sequence
  A synthetic sequence
aaaaaataaaaaaccccctcttaca

SEQ ID NO: 37 - DNA, 21 bp, Artificial Sequence
  A synthetic sequence
gtgtatttggaaggtaggggg

SEQ ID NO: 38 - DNA, 25 bp, Artificial Sequence
  A synthetic sequence
ccctaaacccctaaacctataccat

SEQ ID NO: 39 - DNA, 25 bp, Artificial Sequence
  A synthetic sequence
tgatggtttggagagtatgtaggtt

SEQ ID NO: 40 - DNA, 25 bp, Artificial Sequence
  A synthetic sequence
ttacccctcaactacttaaaaaaca

SEQ ID NO: 41 - DNA, 26 bp, Artificial Sequence
  A synthetic sequence
ggttagtttgattaagggtgaggtta

SEQ ID NO: 42 - DNA, 25 bp, Artificial Sequence
  A synthetic sequence
ccctcaataatcacccaatttctat

SEQ ID NO: 43 - DNA, 25 bp, Artificial Sequence
  A synthetic sequence
ggtggttaagtgtaataggaaatgg

SEQ ID NO: 44 - DNA, 26 bp, Artificial Sequence
  A synthetic sequence
attaccccacaaccttacctcaccta

SEQ ID NO: 45 - DNA, 27 bp, Artificial Sequence
  A synthetic sequence
ggggtgttagagataattagtggagtt

SEQ ID NO: 46 - DNA, 25 bp, Artificial Sequence
  A synthetic sequence
aacaaaaaccaaaacaaataaacca

SEQ ID NO: 47 - DNA, 25 bp, Artificial Sequence
  A synthetic sequence
gtgatggatgttggtaaggtttagt

SEQ ID NO: 48 - DNA, 25 bp, Artificial Sequence
  A synthetic sequence
ttaaaaccttaaaatccaaaaaccc

SEQ ID NO: 49 - DNA, 28 bp, Artificial Sequence
  A synthetic sequence
tttttagggaatgttagattttattaag

SEQ ID NO: 50 - DNA, 25 bp, Artificial Sequence
  A synthetic sequence
ccttctctatccacttaaaaccctt

SEQ ID NO: 51 - DNA, 25 bp, Artificial Sequence
  A synthetic sequence
aagagggggttataaagtttagggt

SEQ ID NO: 52 - DNA, 29 bp, Artificial Sequence
  A synthetic sequence
caatatctacaaatctacatctcctcaaa

SEQ ID NO: 53 - DNA, 27 bp, Artificial Sequence
  A synthetic sequence
ggattattttaattggtaagatttggg

SEQ ID NO: 54 - DNA, 29 bp, Artificial Sequence
  A synthetic sequence
caacaacaatatataaaaattatcacaac

SEQ ID NO: 55 - DNA, 25 bp, Artificial Sequence
  A synthetic sequence
gttgtgtgattgtgttaattttttt

SEQ ID NO: 56 - DNA, 28 bp, Artificial Sequence
  A synthetic sequence
ttttaacccaaaactaaaaactctacct

SEQ ID NO: 57 - DNA, 23 bp, Artificial Sequence
  A synthetic sequence
agggggttttggggagatattta

SEQ ID NO: 58 - DNA, 27 bp, Artificial Sequence
  A synthetic sequence
ccaaaaatcaactacaaacaactaaaa

(19) United States
(12) Patent Application Publication
(10) Pub. No.: US 2023/0053688 A1
(43) Pub. Date: Feb. 23, 2023

(54) METHODS TO ALTER LATENCY IN EBV+ MALIGNANCIES

(71) Applicant: Cornell University, Ithaca, NY (US)

(72) Inventors: Lisa G. Roth, Scarsdale, NY (US); Ethel Cesarman, Jersey City, NJ (US)

(21) Appl. No.: 17/791,754

(22) PCT Filed: Jan. 8, 2021
(86) PCT No.: PCT/US2021/012655

§ 371 Date: Jul. 8, 2022

Related U.S. Application Data
(60) Provisional application No. 62/959,510, filed on Jan. 10, 2020.

(51) Int. Cl. A61K 31/706; A61P 35/00; A61P 31/22; A61K 39/245; G01N 33/50; C12N 7/00

(52) U.S. Cl. CPC A61K 31/706; A61P 35/00; A61P 31/22; A61K 39/245; G01N 33/502; C12N 7/00; C12N 2710/16271; C12N 2710/16234

(57) ABSTRACT

Methods of altering viral latency, sensitizing EBV+ tumors, or modulating viral immunogenicity, are provided.

### CROSS-REFERENCE TO RELATED APPLICATIONS

This application claims the benefit of the filing date of U.S. application No. 62/959,510, filed on Jan. 10, 2020, the disclosure of which is incorporated by reference herein.

### STATEMENT OF GOVERNMENT RIGHTS

This invention was made with government support under grant K08CA219473 awarded by the National Institutes of Health. The government has certain rights in the invention.

### BACKGROUND

The gamma herpes virus EBV is implicated in a variety of malignancies including aggressive B-cell lymphomas (Cesarman, 2014). 200,000 Epstein-Barr virus associated malignancies occur worldwide annually (Cohen et al., 2011; McLaughlin et al., 2008). Three main latency patterns have been described in EBV, which correlate with the immune status of the patient and expression of immunogenic EBV proteins (Carbone et al. 2008). In latency I, the EBV nuclear antigen 1 (EBNA1) and EBV-encoded small RNAs (EBERs) are expressed, in addition to some microRNAs. In contrast, latency III tumors have unrestricted expression of all EBV-encoded latent nuclear antigens (e.g., EBNA1, EBNA2, EBNA3A-C, and LP) and latent membrane proteins (e.g., LMP1, LMP2A, LMP2B). Latency III proteins are highly immunogenic, and this program only persists in severely immunocompromised hosts. Latency II is intermediate with respect to expression of EBNA1 and the latent membrane proteins.

Common EBV-associated lymphomas include Burkitt lymphoma (BL) and HIV-associated diffuse large B-cell lymphoma (HIV-DLBCL), in which the single Epstein-Barr nuclear antigen EBNA1 is produced. EBNA1 is poorly immunogenic, enabling BL and DLBCL to evade otherwise promising cytotoxic T-lymphocyte (CTL) therapeutic approaches. In particular, in EBV+BL and HIV-DLBCL, EBV exists in a latency I pattern, allowing the tumor to evade the immune response to EBV (Burkitt 1958, Arvey, Ojesina et al. 2015). In contrast, EBV+post-transplant proliferative disorder (PITLD) exhibits a latency III profile where the virus expresses its entire latency gene complex (10 latency proteins and two small RNAs). PTLD arises in the context of severe host immune suppression after solid organ or hematopoietic stem cell transplant (LaCasce 2006). Since the latency III program is highly immunogenic, PLD can often be eradicated with restoration of the host immune response through reduction of immunosuppressive therapy (Dierickx, Tousseyn et al. 2015). PTLD has also been successfully treated with ex vivo derived EBV-specific cytotoxic T-lymphocytes (EBV-CTLs) (Prockop, Doubrovina et al., Haque, Wilkie et al. 2002, Haque, Wilkie et al. 2007, Barker, Doubrovina et al. 2010, Doubrovina, Oflaz-Sozmen et al. 2012). EBV-CTLs are generated from healthy donors using EBV-transformed B-lymphoblastoid cell lines as antigen presenting cells. More recently, third party EBV-CTLs have been utilized through the generation of HLA-typed EBV-specific T-cell line banks. Similarly, latency II tumors have been successfully treated with EBV-CTLs directed against the latency II/III antigen LMP1 (Bollard, Gottschalk et al. 2014). This therapeutic approach fails, however, in latency I EBV+ tumors because they express a limited set of viral antigens that are not immunogenic such as immunogenic latency II/III viral antigens.

### SUMMARY

A high throughput drug screen revealed that agents including hypomethylating agents, e.g., 5-azacytadine or decitabine, and other epigenetic modifiers, e.g., proteasome inhibitors, and agents involved in modulation of cell cycle or DNA damage response, induced latency II/III in latency I tumors. Furthermore, this conversion sensitized tumors to T-cell mediated cell killing. Thus, pharmacologic conversion of latency I EBV+ tumors to latency II/III may be employed to sensitize resistant cells to T-cell mediated killing. As a result, those converted tumors cancers may be more sensitive to immunotherapies, for instance, EBV-specific cytotoxic T-cells in the patient or exogenously administered EBV-specific cytotoxic T-cells.

In one embodiment, a method to convert EBV latency I tumors in a mammal to EBV latency II/III tumors is provided. The method includes, in one embodiment, administering to the mammal a composition comprising an effective amount of a hypomethylating agent, e.g., a DNA methyltransferase (DNMT) inhibitor. In one embodiment, the method includes administering to the mammal a composition comprising an effective amount of one or more agents that increase expression of LMP1, LMP2A, LMP2B, EBNA2, EBNA3A, EBNA3B, EBNA3C, or any combination thereof. In one embodiment, the method includes administering to the mammal a composition comprising an effective amount of one or more epigenetic modifying agents. In one embodiment, the method includes administering to the mammal a composition comprising an effective amount of one or more hypomethylating agents. In one embodiment, the hypomethylating agent comprises a cytidine nucleoside analog, e.g., azacytidine, 5,6-dihydro-5-azacytidine, 5-aza-2′-deoxycytidine (5-AZA-CdR, decitabine), S110 (Lavelle et al., J. Transl. Med., 8:92 (2010)), 2′,2′-difluorodeoxycytidine (dFdC), or guadecitabine. In one embodiment, the hypomethylating agent comprises an azanucleoside. In one embodiment, one or more agents involved in modulation of cell cycle or DNA damage response are employed. In one embodiment a combination of any of a hypomethylating agent, an epigenetic modifier, a proteasome inhibitor, or an agent involved in modulation of cell cycle or DNA damage response, may be employed. In one embodiment, any agent, or any combination of agents, listed in Table 2 may be employed. In one embodiment, one or more proteasome inhibitors are employed. In one embodiment, one or more methyltransferase inhibitors are employed. In one embodiment, the agent increases EBV latency III gene expression by at least >2, >5 or >10 fold. In one embodiment, the agent induces increased EBV latency III gene expression in >70% of tumor cells. In one embodiment, the mammal is a human. In one embodiment, the mammal has Burkitt's lymphoma. In one embodiment, the mammal has diffuse large B-cell lymphoma (DLBCL). In one embodiment, the mammal has Hodgkin lymphoma. In one embodiment, the mammal has nasopharyngeal cancer or gastric cancer.

In one embodiment, a method to treat EBV+ tumors in a mammal is provided that includes administering to a mammal having a latency I EBV+ tumor a composition comprising an effective amount of an agent. The method includes, in one embodiment, administering to the mammal a composition comprising an effective amount of a hypomethylating agent. In one embodiment, the hypomethylating agent comprises a cytidine nucleoside analog, e.g., azacytidine, 5,6-dihydro-5-azacytidine, 5-aza-2′-deoxycytidine (5-AZA-CdR, decitabine), S110, 2′,2′-difluorodeoxycytidine (dFdC), or guadecitabine. In one embodiment, the hypomethylating agent or DNMT inhibitor comprises GSK3685032, GSK3484862, NSC-319745, NSC-106084, NSC-14778, CC-486, CM272, RG108, nanamycin A, a maleimide containing molecule or derivative, CP-4200, 4′-thio-2′deoxycytidine, 5′-fluoro-2′deoxycytidine, procaine, procainamide, 5175328, laccaic acid A, SGI-1027, RG108-1, EGCG, genistein, SW155246, quinoline containing compounds, GSK3482364, GSK3484862, SGI-100, methamphetamine, disulfiram, zebularine, SW155246, OR-2003, OR-2100 or hypomethylating agents or DNMT inhibitors disclosed in Zhou et al., Cur. Topics Med. Chem., 18:2448 (2018)), Wee et al., Anticancer Res. 12:759 (2019)), Sharma et al., Mol. Carcino., 55:1843 (2016)), Castillo-Aguilera et al., Biomolecules, 7:3 (2017)), Yuan et al., Bioorg. Chem., 87:200 (2019)), Kang et al., Invest. New Drugs 37:1158 (2019)), Tao et al., Nucl. Acids Res., 39:9508 (2011)), or Hattori et al., Clin. Epigenet., 11:111 (2019)), the disclosures of which are incorporated by reference herein. In one embodiment, one or more agents that increase expression of LMP1, LMP2A, LMP2B, EBNA2, EBNA3A, EBNA3B, EBNA3C, or any combination thereof, may be employed to treat EBV+ tumors. In one embodiment, one or more epigenetic modifying agents are employed. In one embodiment, one or more hypomethylating agents are employed. In one embodiment, one or more proteasome inhibitors are employed. In one embodiment, one or more agents involved in modulation of cell cycle or DNA damage response are employed. In one embodiment, one or more methyltransferase inhibitors are employed. In one embodiment, a combination of any of a hypomethylating agent, an epigenetic modifier, a proteasome inhibitor, or an agent involved in modulation of cell cycle or DNA damage response, may be employed. In one embodiment, any agent, or any combination of agents, listed in Table 2 may be employed. In one embodiment, the mammal is a human. In one embodiment, the mammal has EBV+ lymphoma. In one embodiment, the mammal has Burkitt's lymphoma. In one embodiment, the mammal has diffuse large B-cell lymphoma (DLBCL). In one embodiment, the mammal has Hodgkin lymphoma. In one embodiment, the mammal has nasopharyngeal cancer or gastric cancer. In one embodiment, the mammal is further administered an immunomodulatory agent, e.g., a checkpoint inhibitor, an EZH2 inhibitor, e.g., tazemetostat, CPI-1205, GSK 2816126, SHR2554, CPI-0209, PF-06821497, or DS-32016, or EBV-specific cytotoxic T cells, e.g., after at least some tumor cells in latency I are induced to latency II/III. In one embodiment, prior to administration, a physiological sample of a mammal, e.g., a blood sample or tumor biopsy, is analyzed for the presence or amount of tumor cells in latency I. In one embodiment, mammals having tumor cells in latency I, e.g., at least 10%, 30%, 50%, 70% or more tumor cells in latency I relative to, e.g., latency II/III, are administered an effective amount of a hypomethylating agent or DNMT inhibitor. In one embodiment, the hypomethylating agent or DNMT inhibitor is administered for 1, 2, 3, 4 or 5 days. In one embodiment, a hypomethylating agent or DNMT inhibitor is orally administered to a subject. In one embodiment, a hypomethylating agent or DNMT inhibitor is intravenously administered to a subject. In one embodiment, a subject is infused with hypomethylating agent or DNMT inhibitor. In one embodiment, the hypomethylating agent or DNMT inhibitor is administered at 10 to 20 mg/m², e.g., every 8, 12 or 24 hours. In one embodiment, the hypomethylating agent or DNMT inhibitor is administered at 30 to 60 mg/m²/day. In one embodiment, the hypomethylating agent or DNMT inhibitor is administered at 20 to 40 mg/m², e.g., every 8, 12 or 24 hours. In one embodiment, the hypomethylating agent or DNMT inhibitor is administered at 60 to 120 mg/m²/day. For example, a subject is administered via infusion a hypomethylating agent or DNMT inhibitor at 15 mg/m², e.g., every 8 to 12 hours or per day, for 3 days. In one embodiment, a subject is administered via infusion a hypomethylating agent or DNMT inhibitor at 20 mg/m², e.g., every 8 to 12 hours or per day, for 5 days. In one embodiment, the conversion of tumor cells from latency I to latency II/III is monitored in biopsy samples.

In one embodiment, a method to sensitize EBV+ tumors in a mammal to T-cell mediated killing is provided that includes administering to the mammal a composition comprising an effective amount of an agent. The method includes, in one embodiment, administering to the mammal a composition comprising an effective amount of a hypomethylating agent. In one embodiment, the hypomethylating agent comprises a cytidine nucleoside analog, e.g., azacytidine, 5,6-dihydro-5-azacytidine, 5-aza-2′-deoxycytidine (5-AZA-CdR, decitabine), S110, 2′,2′-difluorodeoxycytidine (dFdC), or guadecitabine. In one embodiment, one or more agents that increase expression of LMP1, LMP2A, LMP2B, EBNA2, EBNA3A, EBNA3B, EBNA3C, or any combination thereof, may be employed to sensitize EBV+ tumors. In one embodiment, one or more epigenetic modifying agents are employed. In one embodiment, one or more hypomethylating agents are employed. In one embodiment, one or more proteasome inhibitors are employed. In one embodiment, one or more agents involved in modulation of cell cycle or DNA damage response are employed. In one embodiment, one or more methyltransferase inhibitors are employed. In one embodiment a combination of any of a hypomethylating agent, an epigenetic modifier, a proteasome inhibitor, or an agent involved in modulation of cell cycle or DNA damage response, may be employed. In one embodiment, any agent, or any combination of agents, listed in Table 2 may be employed. In one embodiment, the mammal is a human. In one embodiment, the mammal has Burkitt's lymphoma. In one embodiment, the mammal has diffuse large B-cell lymphoma (DLBCL). In one embodiment, the mammal has Hodgkin lymphoma. In one embodiment, the mammal has nasopharyngeal cancer or gastric cancer. In one embodiment, prior to administration, a physiological sample of a mammal, e.g., a blood sample or tumor biopsy is analyzed for the presence or amount of tumor cells in latency I.

In one embodiment, a method to modulate viral immunogenicity in a mammal having EBV+ lymphoma is provided. The method includes administering to the mammal a composition comprising an effective amount of, in one embodiment, a hypomethylating agent. In one embodiment, the mammal is a human. In one embodiment, the mammal has Burkitt's lymphoma. In one embodiment, the mammal has diffuse large B-cell lymphoma (DLBCL). In one embodiment, the mammal has Hodgkin lymphoma. In one embodiment, the mammal has nasopharyngeal cancer or gastric cancer. In one embodiment, one or more agents that increase expression of LMP1, LMP2A, LMP2B, EBNA2, EBNA3A, EBNA3B, EBNA3C, or any combination thereof, may be employed. In one embodiment, one or more proteasome inhibitors are employed. In one embodiment, one or more epigenetic modifying agents are employed. In one embodiment, one or more hypomethylating agents are employed. In one embodiment, one or more agents involved in modulation of cell cycle or DNA damage response are employed. In one embodiment, one or more methyltransferase inhibitors are employed. In one embodiment a combination of any of a hypomethylating agent, an epigenetic modifier, a proteasome inhibitor, or an agent involved in modulation of cell cycle or DNA damage response, may be employed. In one embodiment, any agent, or any combination of agents, listed in Table 2 may be employed. In one embodiment, prior to administration, a physiological sample of a mammal, e.g., a blood sample or tumor biopsy, is analyzed for the presence or amount of tumor cells in latency I.

In one embodiment, the method further includes administering one or more immune modulators, e.g., to enhance the immune response (immunotherapy). Immune modulators useful in the methods include but are not limited to PD-1/PD-L1 and CTLA-4 inhibitors, for example, pembrolizumab, nivolumab, REGN2810, BMS-936558, SHR1210, IBI308, PDR001, Anti-PD-1, BGB-A317, BCD-100 or JS001 (anti-PD-1), ipilimumab or tremelimumab (anti-CTLA-4), or avelumab, atezolizumab, durvalumab, or KN035 (Anti-PD-L1) or CTLs. In one embodiment, the CTLs that are administered are allogeneic. In one embodiment, the CTLs that are administered are autologous.

Also provided is an assay to detect agents that convert EBV+ latency I cancers to latency II or latency III cancers. In one embodiment, EBV latency I tumor cells are contacted with one or more agents; and an agent that converts the EBV latency I tumor cells to EBV latency II/III tumor cells, e.g., enhances expression of LMP1, LMP2A, LMP2B, EBNA2, EBNA3A, EBNA3B, EBNA3C, or any combination thereof, is detected. In one embodiment, protein expression is detected. In one embodiment RNA expression is detected. In one embodiment, dose dependent induction of LMP1 or Cp transcripts is detected at doses as low as 25 nM. In one embodiment, the agent that is detected is a hypomethylating agent. In one embodiment, the agent induces induction of LMP1 and Cp at doses <1 μM. In one embodiment, the agent is not 5-azacytidine. In one embodiment, expression of LMP1 and EBNA3C is detected.

### BRIEF DESCRIPTION OF FIGURES

FIG. 1: High throughput drug screen identifies pharmacologic agents that induce latency III antigen expression. A) Immunoblot of BL cell lines to characterize latency. BC2: latency I control, LCL9001: latency III control, Ramos: EBV negative control; B) Heatmap showing the fold change in LMP1 for two replicates across 443 compounds. Dendrogram branches on the right illustrate groupings based on unsupervised clustering, highlighting a cluster of 33 compounds inducing a greater than 2-fold change in both replicates (blue branches). Inset shows the list of 33 compounds grouped based on similarity of pathway targets; C) Network plot showing the pathway enrichments based on drug targets. Each node denotes a sub-pathway, with colors delineating pathway groupings (see table). Nodes with multiple colors denote shared pathway groupings; D) Focused screen of epigenetic modifying agents. qRT-PCR for Cp and LMP1 promoter transcripts in cells were treated with drug vs. vehicle control for 48 hours. Data is shown as fold change in treated cells compared to vehicle control. Experiments were performed in duplicate. Drug doses were as follows: GSK-126 (5 μM), EPZ-6438 (5M), romidepsin (0.25 nM), HDAC3i (5 μM), 5-azacytidine (4M), decitabine (1 μM). Error bars represent SEM.

FIG. 2: Hypomethylating agents induce immunogenic EBV antigens. A, C) qRT-PCR for Cp and LMP1 promoter in cells were treated with drug (decitabine or 5-azacytadine) vs. vehicle control for 48 hours at the following doses listed from L to R: vehicle, 10 nM, 25 nM, 50 nM, 100 nM, 250 nM, 500 nM, 1000 nM. Data is shown as fold change in treated cells compared to vehicle control. Experiments were performed in triplicate. Error bars represent SEM. B, D) Immunoblot for viral proteins as indicated. BL cells were incubated with drug at the indicated doses for 48 hours. LCL-9001 is a latency III positive control. BC2 is a latency I control. Ramos is an EBV-negative BL used as a negative control. Lower panel in 2D represents a longer exposure time for LMP1. E-F) Immunohistochemistry for EBNA2 and LMP1 in cell blocks generated from Mutu I, Kem I, and Rael cells treated as indicated. Cells were exposed to 5-Aza at 4 uM, decitabine at 500 nM, or vehicle control for 48 hours. Experiments were performed in triplicate. Representative images were obtained on an Olympus BX 43 microscope. Camera: Jenoptik ProgResCF; software: ProgRes Mac Capture Pro, 2013. Original magnification×600 with 60/0.80 objective lens. G-F) Image quantification using HALO (Indica labs). Error bars: SEM. *=p-value <0.05, ***=p-value <0.001, ****=p-value <0.0001.

FIG. 3: Decitabine induces expression of viral antigens in BL xenograft models. A-B) Immunohistochemistry for EBNA2 and LMP1 in tumors obtained from Mutu I, Kem I or Rael xenograft mice as indicated. Experiments were performed with 2 mice/condition/cell line for each of the following conditions: vehicle treatment, decitabine 0.5 mg/kg intraperitoneally (IP) daily, decitabine 1 mg/kg IP daily. Representative images were obtained on an Olympus BX 43 microscope. Camera: Jenoptik ProgResCF; software: ProgRes Mac Capture Pro, 2013. Original magnification×600 with 60/0.80 objective lens. C-D) Image quantification using HALO (Indica labs). Error bars: SEM.

FIG. 4: Decitabine induction of viral antigens persists after removal of drug. A) qRT-PCR for LMP1 and Cp in cells were treated with 250 nM decitabine vs. vehicle control for 72 hours and then evaluated after removal of drug at the indicated timepoints. Data is shown as fold change in treated cells compared to vehicle control. Experiments were performed in triplicate. Error bars represent SEM. B) Quantification of EBNA2 positive cells in Rael xenograft tumors as indicated. Error bars: SEM. *=p-value <0.05, **=p-value <0.01. C) IHC for EBNA2 on Rael xenograft tumors after treatment with decitabine or vehicle at the specified timepoints. Mice were treated with decitabine or vehicle control and then evaluated immediately after treatment (n=4), 4 days after discontinuation of drug (n=4) or at the time of sacrifice due to progressive tumor (n=8). Microscope: Olympus BX 43 microscope. Camera: Jenoptik ProgResCF; software: ProgRes Mac Capture Pro, 2013. Original magnification×600 with 60/0.80 objective lens.

FIG. 5: Global EBV DNA hypomethylation is observed after decitabine treatment in latency I EBV+BL. A) EBV genome plot with positions targeted for DNA methylation analysis using MassARRRAY indicated (red numbers). Twenty-eight regions were selected across the genome (1-13 CpGs per region) representing primarily EBV gene promoters. Latent and lytic genes are indicated in brown and blue, respectively. “Capture region” illustrates the area of coverage for Methyl-capture sequencing. B) Heatmap of quantitative DNA methylation levels in vehicle- and decitabine-treated cells. Regions are ordered according to positions in (A) (white; unmethylated, blue; methylated, grey; no data). C) Heatmap of methylation of CpGs, n=1,022 from methyl-capture sequencing. Washout=cells treated with drug×48 hours followed by 7 days of incubation in media without drug.

FIG. 6: Localization of differentially methylated CpGs in decitabine treated BL cell lines and xenografts. Decitabine and vehicle treated cells and xenograft tumors were evaluated with Methyl-Capture sequencing as described above. Differentially methylated areas were mapped to the EBV genome using Integrative Genomics Viewer (Broad Institute, https://software.broadinstitute.org/software/igv). DCB: decitabine, DMC: differentially methylated cytosines.

FIG. 7: Decitabine treatment results in T-cell mediated lysis in-vitro and T-cell trafficking to tumors in-vivo. A-C) Cr release assay in the indicated cell lines incubated with EBV-CTLs reactive to EBNA3C, EBNA3A or LMP1 as labeled. BL cells were treated with decitabine at 250 nM or vehicle control for 72 hours. Controls are as follows: (A) autologous dendritic cells with A0201 HLA loaded with EBNA3C peptide (positive control) and autologous dendritic cells with A0201 HLA alone (negative control); (B) EBV-transformed autologous BLCL (positive control) and autologous dendritic cells (negative control); (C) EBV-transformed autologous BLCL (positive control) and autologous PHA-activated blasts (negative control). D-E) IHC for EBNA2 and CD8 in xenograft tumors as indicated. Microscope: Olympus BX 43 microscope. Camera: Jenoptik ProgResCF; software: ProgRes Mac Capture Pro, 2013. Original magnification×600 with 60/0.80 objective lens. F) Bioluminescence in Rael-luc xenografts. G) HALO quantification of CD8 in Mutu I xenografts in the indicted treatment cohorts. Upon engraftment, Mutu I xenograft mice were randomized to treatment with decitabine at 1 mg/kg daily×3 days or vehicle control followed by EBV-CTLs twice weekly vs. control with 4 mice in each cohort. Mice were humanely sacrificed at the time of tumor growth >2000 mm³ or at day 18 to evaluate for T-cell trafficking to tumor.

FIG. 8: Cell viability after exposure to hypomethylating agents. BL cell lines were exposed to decitabine or 5-azacytadine for 48 hours at a range of doses as follows (from L to R): 0, 500 nM, 1 uM, 2 uM, 4 uM, and 9 uM for 5-azacytidine and 0, 5 nM, 50 nM, 500 nM, 5 uM, 50 uM for decitabine. Cell viability was measured using Cell Titer-Glo. Arrows indicate the dose at which maximal induction of LMP1/Cp transcripts were observed.

FIG. 9: Evaluation of lytic induction after treatment with decitabine. A) Fold change in BZLF1 for BL cells treated with decitabine×72 hours and evaluated at the indicated timepoints. Experiment performed in triplicate. B) Upper panel: Immunohistochemistry for BZLF1 in BL xenografts treated at the indicated doses. Lowe panel: HALO quantification of BZLF1 expression by IHC in xenograft tumors. Mice were treated with vehicle or decitabine at the indicated doses×7 days. Tumors were harvested on day 7. Representative images were obtained on an Olympus BX 43 microscope. Camera: Jenoptik ProgResCF; software: ProgRes Mac Capture Pro, 2013.

FIG. 10: Evaluation of decitabine followed by EBV-CTLs in-vivo in Rael xenografts. A, C) In-vivo imaging of Rael-luc xenograft mice; B) LMP1 and BZLF1 quantification in Rael xenograft tumors obtained after 7 days of treatment with vehicle or decitabine at 1 mg/kg. DCB: decitabine. D) Immunohistochemistry for PD-1 in each of the 4 conditions as indicated. Representative images were obtained on an Olympus BX 43 microscope. Camera: Jenoptik ProgResCF; software: ProgRes Mac Capture Pro, 2013.

FIG. 11: Decitabine induces T-cell homing in Mutu I xenografts. IHC in Mutu I xenograft tumors in the treatment cohorts listed. Microscope: Olympus BX 43 microscope. Camera: Jenoptik ProgResCF; software: ProgRes Mac Capture Pro, 2013. Original magnification×600 with 60/0.80 objective lens.

### DETAILED DESCRIPTION

Three main latency patterns have been described in EBV-associated malignancies, which correlate with immune status of the patient and expression of immunogenic EBV proteins. In latency I, the EBV nuclear antigen1 (EBNA1) and EBV-encoded small RNAs (EBERs) are expressed. In contrast, latency III tumors have unrestricted expression of all EBV-encoded nuclear antigens (e.g., EBNA1, EBNA2, EBNA3A-C, and LP) and latent membrane proteins (e.g., LMP1, LMP2A, LPM2B). These proteins are highly immunogenic, so latency III occurs in severely immunocompromised individuals. Cellular therapy directed at EBV is effective in the post-transplant setting in latency III tumors. BL and HIV-associated DLBCL, however, express a latency I pattern and are resistant to EBV-specific cellular therapies.

Despite advances in T-cell immunotherapy against EBV-infected lymphomas that express the full EBV latency III program, a barrier has been that most EBV+ lymphomas express the latency I program, in which the single Epstein-Barr nuclear antigen (EBNA1) is produced. EBNA1 is poorly immunogenic, enabling tumors to evade immune responses. The present disclosure provides for methods that employ agents that convert latency I EBV+ malignancies to latency II/III and so sensitize to tumors to T-cell mediated killing (e.g., lysis), e.g., by the patient's own T cells or autologous CTLs. Thus, epigenetic reprogramming sensitizes immunologically silent EBV+ lymphomas to viral directed immunotherapy.

As described herein below, using a high throughput screen, agents including decitabine (5-aza-2′-deoxycytidine) and 5-azacytadine were identified as inducing latency II/III antigen expression in latency I EBV+ Burkitt lymphoma, e.g., inducers of immunogenic EBV antigens including LMP1, EBNA2 and EBNA3C. Induction by decitabine occurred at low doses than decitabine (5-aza-2′-deoxycytidine) induced latency II/III in a higher percentage of cells than 5-azacytadine and at lower concentrations, and persisted after removal of decitabine. Moreover, decitabine treatment of latency I EBV+ Burkitt lymphoma sensitized cells to lysis by EBV-specific cytotoxic T-cells (EBV-CTLs). In latency I Burkitt lymphoma xenografts, decitabine followed by EBV-CTLs resulted in T-cell homing to tumors and inhibition of tumor growth. Collectively, these results identify key epigenetic factors required for latency restriction and highlight a therapeutic approach to sensitize EBV+ lymphomas to immunotherapy. Thus, in one embodiment, the method includes decitabine pre-treatment, which converts latency I EBV+ lymphomas to latency II/III and sensitizes cells to T-cell mediated cell death, e.g., with third party EBV-specific T-lymphocytes.

### Compositions for Use in the Methods

The methods employ compositions containing agents, e.g., epigenetic reprogramming agents and/or immunomodulators, such as T cells, e.g., CTLs. Thus, the agent(s) that is/are administered may be, but are not limited to, a small molecule, an antibody, cells, or a combination thereof. The compositions can be pharmaceutical compositions. In some embodiments, the compositions can include a pharmaceutically acceptable carrier. By “pharmaceutically acceptable” it is meant that a carrier, diluent, excipient, and/or salt is compatible with the other ingredients of the formulation, and not deleterious to the recipient thereof.

The composition can be formulated in any convenient form. In some embodiments, the compositions can include one or more small molecules or one or more antibody types.

In some embodiments, the therapeutic agents (e.g., each type of small molecule or antibody or cell), are administered in a “therapeutically effective amount.” Such a therapeutically effective amount is an amount sufficient to obtain the desired physiological effect, such as conversion of EBV+ latency I tumors to EBV+ latency II/III tumors, or inhibition or treatment of EBV+ tumors. For example, in some cases the therapeutic agents can convert at least 5%, or 10%, or 15%, or 20%, or 25%, or 30%, or 35%, or 40%, or 45%, or 50%, or 55%, or 60%, or 65%, or %70, or 80%, or 90%, 95%, or 97%, or 99%, or any numerical percentage between 5% and 100%, of EBV+ latency I tumor cells to latency II/III cells. In some cases, the therapeutic agents can increase expression of LMP1, LMP2A, LMP2B, EBNA2, EBNA3A, EBNA3B, EBNA3C, or any combination thereof, in EBV+ latency I tumor cells by at least 5%, or 10%, or 15%, or 20%, or 25%, or 30%, or 35%, or 40%, or any numerical percentage between 5% and 40%. Administration of therapeutic agents described herein can increase CTL activity, e.g., endogenous CTLs or exogenously administered CTLs, by at least 5%, or 10%, or 15%, or 20%, or 25%, or 30%, or 35%, or 40%, or 45%, or 50%, or 55%, or 60%, or 65%, or %70, or 80%, or 90%, 95%, or 97%, or 99%, or any numerical percentage between 5% and 100%. Such increases are relative to corresponding cells without treatment with the therapeutic agent(s).

To achieve the desired effect(s), the therapeutic agents may be administered as single or divided dosages. For example, therapeutic agents can be administered in dosages of at least about 0.01 mg/kg to about 500 to 750 mg/kg, of at least about 0.01 mg/kg to about 300 to 500 mg/kg, at least about 0.1 mg/kg to about 100 to 300 mg/kg or at least about 1 mg/kg to about 50 to 100 mg/kg of body weight, although other dosages may provide beneficial results. The amount administered will vary depending on various factors including, but not limited to, the type of small molecule, cell, antibody, or combination thereof chosen for administration, the extent or duration of disease, the weight, the physical condition, the health, and the age of the subject animal. Such factors can be readily determined by the clinician employing animal models or other test systems that are available in the art.

Thus, administration of the therapeutic agents in accordance with the present invention may be in a single dose, in multiple doses, in a continuous or intermittent manner, depending, for example, upon the recipient's physiological condition, whether the purpose of the administration is therapeutic or prophylactic, and other factors known to skilled practitioners. The administration of the therapeutic agents and compositions may be essentially continuous over a preselected period of time or may be in a series of spaced doses. Both local and systemic administration is contemplated.

To prepare the composition, small molecules, compounds, antibodies, and/or other agents, e.g., CTLs, are synthesized or otherwise obtained, purified as necessary or desired. These small molecules, compounds, antibodies, and other agents can be suspended in a pharmaceutically acceptable carrier and/or lyophilized or otherwise stabilized. The small molecules, compounds, antibodies, other agents, and combinations thereof, can be adjusted to an appropriate concentration, and optionally combined with other agents. The absolute weight of a given small molecules, compounds, antibodies, and/or other agents included in a unit dose can vary widely. For example, about 0.01 to about 2 g, or about 0.1 to about 500 mg, of at least one compound, molecules, antibody, and/or other agent, or a plurality of compounds, molecules, antibodies, and/or other agents can be administered. Alternatively, the unit dosage can vary from about 0.01 g to about 50 g, from about 0.01 g to about 35 g, from about 0.1 g to about 25 g, from about 0.5 g to about 12 g, from about 0.5 g to about 8 g, from about 0.5 g to about 4 g, or from about 0.5 g to about 2 g. Doses of CTLs may be from 1×10⁶ cells/m² to about 1×10⁹ cells/m², from 1×10⁷ cells/m² to about 1×10⁸ cells/m², from 5×10⁷ cells/m² to about 5×10⁸ cells/m², or from 1×10⁷ cells/m² to about 1×10¹⁹ cells/m².

Daily doses of the therapeutic agents can vary as well. Such daily doses can range, for example, from about 0.1 g/day to about 50 g/day, from about 0.1 g/day to about 25 g/day, from about 0.1 g/day to about 12 g/day, from about 0.5 g/day to about 8 g/day, from about 0.5 g/day to about 4 g/day, and from about 0.5 g/day to about 2 g/day.

It will be appreciated that the amount of therapeutic agent for use in treatment will vary not only with the particular carrier selected but also with the route of administration, and the age and condition of the patient. Ultimately the attendant health care provider can determine proper dosage. In addition, a pharmaceutical composition can be formulated as a single unit dosage form.

Thus, one or more suitable unit dosage forms comprising the therapeutic agent(s) can be administered by a variety of routes including oral, parenteral (including subcutaneous, intravenous, intramuscular and intraperitoneal), rectal, dermal, transdermal, intrathoracic, intrapulmonary and intranasal (respiratory) routes. The therapeutic agent(s) may also be formulated for sustained release (for example, using microencapsulation, see WO 94/07529, and U.S. Pat. No. 4,962,091). The formulations may, where appropriate, be conveniently presented in discrete unit dosage forms and may be prepared by any of the methods available to the pharmaceutical arts. Such methods may include the step of mixing the therapeutic agent with liquid carriers, solid matrices, semi-solid carriers, finely divided solid carriers or combinations thereof, and then, if necessary, introducing or shaping the product into the desired delivery system. For example, the therapeutic agent(s) can be linked to a convenient carrier such as a nanoparticle, albumin, polyalkylene glycol, or be supplied in prodrug form. The therapeutic agent(s), and combinations thereof can be combined with a carrier and/or encapsulated in a delivery vehicle such as a liposome.

The compositions may be prepared in many forms that include aqueous solutions, suspensions, tablets, hard or soft gelatin capsules, and liposomes and other slow-release formulations, such as shaped polymeric gels. Administration can also involve parenteral or local administration of the in an aqueous solution or sustained release vehicle.

Thus, while the therapeutic agent(s) and/or other agents can sometimes be administered in an oral dosage form, that oral dosage form can be formulated so as to protect the small molecules, compounds, antibodies, and combinations thereof from degradation or breakdown before the small molecules, compounds, antibodies, or other agents, and combinations thereof provide therapeutic utility. For example, in some cases the small molecules, compounds, antibodies, and/or other agents can be formulated for release into the intestine after passing through the stomach. Such formulations are described, for example, in U.S. Pat. No. 6,306,434 and in the references contained therein.

Liquid pharmaceutical compositions may be in the form of, for example, aqueous or oily suspensions, solutions, emulsions, syrups or elixirs, dry powders for constitution with water or other suitable vehicle before use. Such liquid pharmaceutical compositions may contain conventional additives such as suspending agents, emulsifying agents, non-aqueous vehicles (which may include edible oils), or preservatives. The pharmaceutical compositions may take such forms as suspensions, solutions, or emulsions in oily or aqueous vehicles, and may contain formulatory agents such as suspending, stabilizing and/or dispersing agents. Suitable carriers include saline solution, encapsulating agents (e.g., liposomes), and other materials. The therapeutic agent(s) and/or other agents can be formulated in dry form (e.g., in freeze-dried form), in the presence or absence of a carrier. If a carrier is desired, the carrier can be included in the pharmaceutical formulation, or can be separately packaged in a separate container, for addition to the inhibitor that is packaged in dry form, in suspension or in soluble concentrated form in a convenient liquid.

Therapeutic agent(s) and/or other agents can be formulated for parenteral administration (e.g., by injection, for example, bolus injection or continuous infusion) and may be presented in unit dosage form in ampoules, prefilled syringes, small volume infusion containers or multi-dose containers with an added preservative.

### Exemplary Routes and Formulations

Administration of compositions having agents that convert EBV+ latency I tumors to latency II/III according to the disclosure can be via any of suitable route of administration, particularly parenterally, for example, orally, intranasal, intravenously, intra-arterially, intraperitoneally, intrathecally, intraventricularly, intrasternally, intracranially, intramuscularly, or subcutaneously. Such administration may be as a single dose or multiple doses, or as a short- or long-duration infusion. Implantable devices (e.g., implantable infusion pumps) may also be employed for the periodic parenteral delivery over time of equivalent or varying dosages of the particular formulation. For such parenteral administration, the therapeutic agent may be formulated as a sterile solution in water or another suitable solvent or mixture of solvents. The solution may contain other substances such as salts, sugars (particularly glucose or mannitol), to make the solution isotonic with blood, buffering agents such as acetic, citric, and/or phosphoric acids and their sodium salts, and preservatives.

The compositions alone or in combination with other active agents can be formulated as pharmaceutical compositions and administered to a vertebrate host, such as a human patient in a variety of forms adapted to the chosen route of administration, e.g., orally or parenterally, by intravenous, intramuscular, topical or subcutaneous routes.

Thus, the compositions having an agent(s) that convert EBV+ latency I tumors to latency II/III may be systemically administered, e.g., orally, in combination with a pharmaceutically acceptable vehicle such as an inert diluent or an assimilable edible carrier. They may be enclosed in hard or soft shell gelatin capsules, may be compressed into tablets, or may be incorporated directly with the food of the vertebrate's diet. For oral therapeutic administration, the composition optionally in combination with another active compound may be combined with one or more excipients and used in the form of ingestible tablets, buccal tablets, troches, capsules, elixirs, suspensions, syrups, wafers, and the like. Such compositions and preparations should contain at least 0.1% of active agent. The percentage of the compositions and preparations may, of course, be varied and may conveniently be between about 2 to about 60% of the weight of a given unit dosage form. The amount of the agent and optionally other active compound in such useful compositions is such that an effective dosage level will be obtained.

The tablets, troches, pills, capsules, and the like may also contain the following: binders such as gum tragacanth, acacia, corn starch or gelatin; excipients such as dicalcium phosphate; a disintegrating agent such as corn starch, potato starch, alginic acid and the like; a lubricant such as magnesium stearate; and a sweetening agent such as sucrose, fructose, lactose or aspartame or a flavoring agent such as peppermint, oil of wintergreen, or cherry flavoring may be added. When the unit dosage form is a capsule, it may contain, in addition to materials of the above type, a liquid carrier, such as a vegetable oil or a polyethylene glycol. Various other materials may be present as coatings or to otherwise modify the physical form of the solid unit dosage form. For instance, tablets, pills, or capsules may be coated with gelatin, wax, shellac or sugar and the like. A syrup or elixir may contain the active agent, sucrose or fructose as a sweetening agent, methyl and propylparabens as preservatives, a dye and flavoring such as cherry or orange flavor. Of course, any material used in preparing any unit dosage form should be pharmaceutically acceptable and substantially non-toxic in the amounts employed. In addition, the agent optionally in combination with another active compound may be incorporated into sustained-release preparations and devices.

The composition having an agent(s) that convert EBV+ latency I tumors to latency II/III optionally in combination with another active compound may also be administered intravenously or intraperitoneally by infusion or injection. Solutions of the agent(s) optionally in combination with another active compound or its salts can be prepared in water, optionally mixed with a nontoxic surfactant. Dispersions can also be prepared in glycerol, liquid polyethylene glycols, triacetin, and mixtures thereof and in oils. Under ordinary conditions of storage and use, these preparations contain a preservative to prevent the growth of microorganisms.

The pharmaceutical dosage forms suitable for injection or infusion can include sterile aqueous solutions or dispersions or sterile powders comprising the active ingredient which are adapted for the extemporaneous preparation of sterile injectable or infusible solutions or dispersions, optionally encapsulated in liposomes. In all cases, the ultimate dosage form should be sterile, fluid and stable under the conditions of manufacture and storage. The liquid carrier or vehicle can be a solvent or liquid dispersion medium comprising, for example, water, ethanol, a polyol (for example, glycerol, propylene glycol, liquid polyethylene glycols, and the like), vegetable oils, nontoxic glyceryl esters, and suitable mixtures thereof. The proper fluidity can be maintained, for example, by the formation of liposomes, by the maintenance of particle size in the case of dispersions or by the use of surfactants. The prevention of the action of microorganisms during storage can be brought about by various antibacterial and antifungal agents, for example, parabens, chlorobutanol, phenol, sorbic acid, thimerosal, and the like. In many cases, it may be useful to include isotonic agents, for example, sugars, buffers or sodium chloride. Prolonged absorption of the injectable compositions can be brought about by the use in the compositions of agents delaying absorption, for example, aluminum monostearate and gelatin.

Sterile injectable solutions are prepared by incorporating agent(s) in the required amount in the appropriate solvent with various of the other ingredients enumerated above, followed by filter sterilization. In the case of sterile powders for the preparation of sterile injectable solutions, one method of preparation includes vacuum drying and the freeze drying techniques, which yield a powder of the active ingredient plus any additional desired ingredient present in the previously sterile-filtered solutions.

For topical administration, the agent(s) optionally in combination with another active compound may be applied in pure form, e.g., when they are liquids.

Useful solid carriers include finely divided solids such as talc, clay, microcrystalline cellulose, silica, alumina and the like. Useful liquid carriers include water, alcohols or glycols or water-alcohol/glycol blends, in which the present agents can be dissolved or dispersed at effective levels, optionally with the aid of non-toxic surfactants. Adjuvants such as fragrances and antimicrobial agents can be added to optimize the properties for a given use. The resultant liquid compositions can be applied from absorbent pads, used to impregnate bandages and other dressings, or sprayed onto the affected area using pump-type or aerosol sprayers.

Thickeners such as synthetic polymers, fatty acids, fatty acid salts and esters, fatty alcohols, modified celluloses or modified mineral materials can also be employed with liquid carriers to form spreadable pastes, gels, ointments, soaps, and the like, for application directly to the skin of the user.

In addition, in one embodiment, the disclosure provides various dosage formulations of the agent(s) optionally in combination with another active compound for inhalation delivery. For example, formulations may be designed for aerosol use in devices such as metered-dose inhalers, dry powder inhalers and nebulizers.

Useful dosages can be determined by comparing their in vitro activity, and in vivo activity in animal models. Methods for the extrapolation of effective dosages in mice, and other animals, to humans are known to the art; for example, see U.S. Pat. No. 4,938,949.

Generally, the concentration of the agent(s) optionally in combination with another active compound in a liquid composition, may be from about 0.1-25 wt-%, e.g., from about 0.5-10 wt-%. The concentration in a semi-solid or solid composition such as a gel or a powder may be be about 0.1-5 wt-%, e.g., about 0.5-2.5 wt-%.

The active ingredient may be administered to achieve peak plasma concentrations of the active agent of, in one embodiment, from about 0.5 to about 75 μM, e.g., about 1 to 50 μM, such as about 2 to about 30 μM. This may be achieved, for example, by the intravenous injection of a 0.05 to 5% solution of the active ingredient, optionally in saline, or orally administered as a bolus containing about 1-100 mg of the active ingredient. Desirable blood levels may be maintained by continuous infusion to provide about 0.01-5.0 mg/kg/hr or by intermittent infusions containing about 0.4-15 mg/kg of the active ingredient(s).

The amount of the agent(s) optionally in combination with another active compound, or an active salt or derivative thereof, for use in treatment may vary not only with the particular salt selected but also with the route of administration, the nature of the condition being treated and the age and condition of the patient and will be ultimately at the discretion of the attendant physician or clinician. In general, however, a suitable dose may be in the range of from about 0.5 to about 100 mg/kg, e.g., from about 10 to about 75 mg/kg of body weight per day, such as 3 to about 50 mg per kilogram body weight of the recipient per day, for instance in the range of 6 to 90 mg/kg/day, e.g., in the range of 15 to 60 mg/kg/day.

The agent(s) optionally in combination with another active compound may be conveniently administered in unit dosage form; for example, containing 5 to 1000 mg, conveniently 10 to 750 mg, most conveniently, 50 to 500 mg of active ingredient per unit dosage form.

The desired dose may conveniently be presented in a single dose or as divided doses administered at appropriate intervals, for example, as two, three, four or more sub-doses per day. The sub-dose itself may be further divided, e.g., into a number of discrete loosely spaced administrations; such as multiple inhalations from an insufflator or by application of a plurality of drops into the eye. The dose, and perhaps the dose frequency, will also vary according to the age, body weight, condition, and response of the individual vertebrate. In general, the total daily dose range for an active agent for the conditions described herein, may be from about 1 mg to about 100 mg, from about 10 mg to about 50 mg, from about 10 mg to about 40 mg, from about 20 mg to about 40 mg, from about 20 mg to about 50 mg, from about 50 mg to about 5000 mg, in single or divided doses. In one embodiment, a daily dose range should be about 100 mg to about 4000 mg, e.g., about 1000-3000 mg, in single or divided doses, e.g., 750 mg every 6 hr of orally administered agent.

### Exemplary Polymer Delivery Vehicles

In one embodiment, the agent(s) may be administered in a delivery vehicle. In one embodiment, the delivery vehicle is a naturally occurring polymer, e.g., formed of materials including but not limited to albumin, collagen, fibrin, alginate, extracellular matrix (ECM), e.g., xenogeneic ECM, hyaluronan (hyaluronic acid), chitosan, gelatin, keratin, potato starch hydrolyzed for use in electrophoresis, or agar-agar (agarose). In one embodiment, the delivery vehicle comprises a hydrogel. In one embodiment, the composition comprises a naturally occurring polymer. Table A provides exemplary materials for delivery vehicles that are formed of naturally occurring polymers and materials for particles.

TABLE A

| Particle class | Materials |
|---|---|
| Natural materials or | Chitosan |
| derivatives | Dextran |
| | Gelatine |
| | Albumin |
| | Alginates |
| | Liposomes |
| | Starch |
| Polymer carriers | Polylactic acid |
| | Poly(cyano)acrylates |
| | Polyethyleneimine |
| | Block copolymers |
| | Polycaprolactone |

An exemplary polycaprolactone is methoxy poly(ethylene glycol)/poly(epsilon caprolactone). An exemplary poly lactic acid is poly(D,L-lactic-co-glycolic)acid (PLGA).

Some examples of materials for particle formation include but are not limited to agar acrylic polymers, polyacrylic acid, poly acryl methacrylate, gelatin, poly(lactic acid), pectin(poly glycolic acid), cellulose derivatives, cellulose acetate phthalate, nitrate, ethyl cellulose, hydroxyl ethyl cellulose, hydroxypropylcellulose, hydroxyl propyl methyl cellulose, hydroxypropylmethylcellulose phthalate, methyl cellulose, sodium carboxymethylcellulose, poly(ortho esters), polyurethanes, poly(ethylene glycol), poly(ethylene vinyl acetate), polydimethylsiloxane, poly(vinyl acetate phthalate), polyvinyl alcohol, polyvinyl pyrollidone, and shellac. Soluble starch and its derivatives for particle preparation include amylodextrin, amylopectin and carboxy methyl starch.

In one embodiment, the polymers in the nanoparticles or microparticles are biodegradable. Examples of biodegradable polymers useful in particles preparation include synthetic polymers, e.g., polyesters, poly(ortho esters), polyanhydrides, or polyphosphazenes; natural polymers including proteins (e.g., collagen, gelatin, and albumin), or polysaccharides (e.g., starch, dextran, hyaluronic acid, and chitosan). For instance, a biocompatible polymer includes poly (lactic) acid (PLA), poly (glycolic acid) (PLGA). Natural polymers that may be employed in particles (or as the delivery vehicle) include but are not limited to albumin, chitin, starch, collagen, chitosan, dextrin, gelatin, hyaluronic acid, dextran, fibrinogen, alginic acid, casein, fibrin, and polyanhydrides.

In one embodiment, the delivery vehicle is a hydrogel. Hydrogels can be classified as those with chemically crosslinked networks having permanent junctions or those with physical networks having transient junctions arising from polymer chain entanglements or physical interactions, e.g., ionic interactions, hydrogen bonds or hydrophobic interactions. Natural materials useful in hydrogels include natural polymers, which are biocompatible, biodegradable, support cellular activities, and include proteins like fibrin, collagen and gelatin, and polysaccharides like starch, alginate and agarose.

In one embodiment, the delivery vehicle comprises inorganic nanoparticles, e.g., calcium phosphate or silica particles; polymers including but not limited to poly(lactic-co-glycolic acid) (PLGA), polylactic acid (PLA), linear and/or branched PEI with differing molecular weights (e.g., 2, 22 and 25 kDa), dendrimers such as polyamidoamine (PAMAM) and polymethoacrylates; lipids including but not limited to cationic liposomes, cationic emulsions, DOTAP, DOTMA, DMRIE, DOSPA, distearoylphosphatidylcholine (DSPC), DOPE, or DC-cholesterol; peptide based vectors including but not limited to Poly-L-lysine or protamine; or poly(β-amino ester), chitosan, PEI-polyethylene glycol, PEI-mannose-dextrose, or DOTAP-cholesterol.

In one embodiment, the delivery vehicle comprises polyethyleneimine (PEI), Polyamidoamine (PAMAM), PEI-PEG, PEI-PEG-mannose, dextran-PEI, OVA conjugate, PLGA microparticles, or PLGA microparticles coated with PAMAM.

Lipids having two linear fatty acid chains, such as DOTMA, DOTAP and SAINT-2, or DODAC, may be employed as a delivery vehicle, as well as tetraalkyl lipid chain surfactant, the dimer of N,N-dioleyl-N,N-dimethylammonium chloride (DODAC). All the trans-orientated lipids regardless of their hydrophobic chain lengths (C_{16:1}, C_{18:1} and C_{20:1}) appear to enhance the transfection efficiency compared with their cis-orientated counterparts.

### EXEMPLARY EMBODIMENTS

In one embodiment, a method to convert EBV⁺ latency I tumors in a mammal to EBV⁺ latency II/III tumors is provided. The method includes administering to a mammal identified as having EBV⁺ latency I tumor a composition comprising an effective amount of one or more hypomethylating agents. In one embodiment, the mammal is a human. In one embodiment, the mammal has EBV⁺ lymphoma. In one embodiment, the mammal has Burkitt's lymphoma. In one embodiment, the mammal has diffuse large B-cell lymphoma (DLBCL). In one embodiment, the mammal has Hodgkin lymphoma. In one embodiment, the mammal has nasopharyngeal cancer or gastric cancer. In one embodiment, the agent increases expression of LMP1, EBNA3C, or both. In one embodiment, the hypomethylating agent comprises decitabine or azacytidine. In one embodiment, the agent is a methyltransferase inhibitor. In one embodiment, the hypomethylating agent is systemically administered. In one embodiment, the hypomethylating agent is orally administered. In one embodiment, the hypomethylating agent is injected. In one embodiment, the method further comprises administering an immunotherapeutic. In one embodiment, the immunotherapeutic comprises EBV-specific cytotoxic T-cells. In one embodiment, the immunotherapeutic is a checkpoint inhibitor. In one embodiment, the immunotherapeutic is injected. In one embodiment, the immunotherapeutic is systemically administered. In one embodiment, the immunotherapeutic is orally administered.

In one embodiment, a method to sensitize EBV+ tumors in a mammal to T-cell mediated killing is provided. The method includes administering to a mammal identified as having EBV⁺ latency I tumor a composition comprising an effective amount of one or more hypomethylating agents. In one embodiment, the mammal is a human. In one embodiment, the mammal has Burkitt's lymphoma. In one embodiment, the mammal has diffuse large B-cell lymphoma (DLBCL). In one embodiment, the mammal has Hodgkin lymphoma. In one embodiment, the mammal has nasopharyngeal cancer or gastric cancer. In one embodiment, the agent increases expression of LMP1, EBNA3C, or both. In one embodiment, the hypomethylating agent comprises decitabine or azacytidine. In one embodiment, the agent is a methyltransferase inhibitor. In one embodiment, the hypomethylating agent is systemically administered. In one embodiment, the hypomethylating agent is orally administered. In one embodiment, the hypomethylating agent is injected. In one embodiment, the method further comprises administering an immunotherapeutic. In one embodiment, the immunotherapeutic comprises EBV-specific cytotoxic T-cells. In one embodiment, the immunotherapeutic is a checkpoint inhibitor. In one embodiment, the immunotherapeutic is injected. In one embodiment, the immunotherapeutic is systemically administered. In one embodiment, the immunotherapeutic is orally administered.

In one embodiment, a method to modulate viral immunogenicity in a mammal having EBV+ lymphoma. The method includes administering to a mammal identified as having EBV+ latency I tumor a composition comprising an effective amount of one or more hypomethylating agents. In one embodiment, the mammal is a human. In one embodiment, the mammal has Burkitt's lymphoma. In one embodiment, the mammal has diffuse large B-cell lymphoma (DLBCL). In one embodiment, the mammal has Hodgkin lymphoma. In one embodiment, the mammal has nasopharyngeal cancer or gastric cancer. In one embodiment, the agent increases expression of LMP1, EBNA3C, or both. In one embodiment, the hypomethylating agent comprises decitabine or azacytidine. In one embodiment, the agent is a methyltransferase inhibitor. In one embodiment, the hypomethylating agent is systemically administered. In one embodiment, the hypomethylating agent is orally administered. In one embodiment, the hypomethylating agent is injected. In one embodiment, the method further comprises administering an immunotherapeutic. In one embodiment, the immunotherapeutic comprises EBV-specific cytotoxic T-cells. In one embodiment, the immunotherapeutic is a checkpoint inhibitor. In one embodiment, the immunotherapeutic is injected. In one embodiment, the immunotherapeutic is systemically administered. In one embodiment, the immunotherapeutic is orally administered.

Also provided is use of a hypomethylating agent or DNA methyl transferase inhibitor to induce latency II/III in EBV⁺ latency I tumor cells.

Further provided is an in vitro method to detect an agent that converts EBV latency I tumor cells to EBV latency II/III tumors, comprising: contacting EBV latency I tumor cells with one or more agents; and determining whether the one or more agents convert the EBV latency I tumor cells to EBV latency II/III tumor cells. In one embodiment, the cells are from a patient having an EBV+ tumor. In one embodiment, the agent increases expression of LMP1, EBNA3C, EBNA3A, LMP2, or any combination thereof. In one embodiment, the agent increases expression of BLZF1. In one embodiment, the agent is a hypomethylating agent, DNA methyl transferase inhibitor or a proteasome inhibitor. In one embodiment, RNA expression of one or more EBV proteins is detected.

In one embodiment, a method to determine the latency status of a mammal having an EBV+ tumor is provided. The method includes obtaining a biopsy sample from a mammal having an EBV+ tumor and subjected to a hypomethylating agent therapy and determining the latency status of EBV+ tumor cells in the sample. In one embodiment, the latency status of the sample of the mammal is compared to a sample obtained at an earlier point in time, e.g., pre-therapy or earlier in therapy.

The invention will be further described by the following non-limiting example.

### Example

It was hypothesized that pharmacologic modulation of latency I tumors could induce immunogenic latent viral antigen expression and that this would sensitize resistant tumors to EBV-directed immunotherapy. As described below, the hypomethylating agent decitabine was identified as a potent inducer of the immunogenic antigens LMP1, EBNA2, and EBNA3C in EBV+BL tumors. Induction of these antigens resulted in homing of EBV-specific T cells into tumor tissues, and sensitized tumor cells to T-cell lysis, suggesting that hypomethylating agents followed by EBV-CTLs may be a therapeutic approach in latency I EBV+ lymphomas.

### Methods

Cell culture, immunoblot, immunohistochemistry, qRT-PCR and reagents: Cell lines were obtained from ATCC or collaborators. Cell typing was confirmed by short tandem repeat profiling performed by Idex Bioresearch (Westbrook, Me.). Cells were used within 3 months of thawing. Cell viability was determined using CellTiter-Glo (Promega) and the GloMax® Multi+ detection system (Promega). IC50 was calculated using Prism6 software. qRT-PCR was performed on the ABI 7500 Fast PCR system (Thermo Fisher Scientific) using Taqman primers and probes for BZLF1, LMP1, and Cp as described Bell et al. (2006). Further details on cell lines, drugs, qRT-PCR methods, and antibodies are outlined below.
High throughput drug screen: Kem I cells were incubated in 100 uL of culture media at indicated drug concentrations. After 48-hours, cells were washed with phosphate-buffered saline and resuspended in TRI Reagent (Zymo Research). RNA was extracted using the Direct-zol-96 RNA kit (Zymo Research). DNase-treated total RNA was reverse transcribed with the High-Capacity cDNA Reverse Transcription Kit (Thermo Fisher Scientific). qRT-PCR was performed as described above.
Xenograft Models: Non-obese diabetic/severe combined immunodeficiency (NOD-SCID) and NSG mice were obtained from Jackson Laboratories. Six to eight-week-old mice were injected subcutaneously in the flank with 1×10⁷ BL cells in PBS with matrigel. Tumors were measured by calipers and/or bioluminescent imaging performed using the IVIS Spectrum, with retroorbital luciferin injections. At sacrifice, tumors were harvested for RNA, DNA, protein, and sectioned for immunohistochemistry.
EBV-CTLs and Cr release assay: EBV-CTLs were generated from peripheral blood mononuclear cells separated by low density separation from peripheral blood of normal consented donors by stimulation with autologous B cells transformed with B95.8 EBV as previously described (Roskrow, Suzuki et al. 1998, Doubrovina, Oflaz-Sozmen et al. 2012).
DNA methylation analysis using MassARRAY and MethylomeCapture: Details are described in supplemental materials and methods. PCR primers specific for EBV are listed in Table 1.

TABLE 1
Primers used and genomic locations for the MassARRAY EBV methylation assay.
EBV genomic loci

| | | | EBV genome |
|---|---|---|---|
| Primer | | | location |
| Name | Forward sequence | Reverse sequence | (NC_007605) |
| Cp | GGGTTGGGTAAAGGGGTTTTA | CCATCTAATCTAAAATTTACAA | 11179-11353 |
| | | CAAAACA | |
| BdRF1 | TTTGTTGTTTAGGTTGGTTTGAAGT | CAAAAATATAACCAATATCCCA | 136300-136543 |
| | | ACC | |
| BFRF1 | TTTGGATAATTTTTTAGAAGTTGAGA | CAAAAACATCAAAAACAAAAC | 47300-47608 |
| | | CAC | |
| BMRF1 | AAGGGTTATTTGGATTTAGGAGTTG | AACCTAACCAATATCACCCAAA | 67245-67404 |
| | | ATA | |
| BZLF1 | GGGGATAATGGAGTTAATATTTAGG | CCAAATTTAAACAACTACTACA | 89971-90179 |
| | | ACACTACC | |
| Zp | GGTTTGATTGGTTTTTTTATTAGGG | ACCCCTACCTACCTCTTTAACT | 91358-91520 |
| | | CC | |
| OriLyt | GGATTTTGGTGTTAGGTAGGGATT | ATATTACACAAAAACCCCAAA | 40392-40562 |
| | | AAAA | |
| OriLyt 2 | TAATAGGGGAAGTAAGGTTTTTTGT | CCAAAACTAAATCCTAAAACCC | 140576-140771 |
| | | AAA | |
| Rp | GGTGTTGTGTTTTGTATGGTATTTTAT | TACCCCAACCAAATATTCAAAA | 93866-94077 |
| | | AC | |
| BBRF1 | GGTTTTTATGAGGTGTTTAAATTGG | TAAACTCTCCCACCCAAACAAA | 101042-101247 |
| RPMS1 | GTAAGTTTAAGTTTGGTGTTGGGGT | CCCTCTCTCTAAAAATTTACAT | 150463-150617 |
| | | TCCA | |
| BARF0 | TTGTAGAAGTTGTTGAAGGAGGTTTT | AAAATCTAACCAAACTACAATC | 159134-159590 |
| | | CTACC | |
| BORF1 | GTTTATTTTTGTTAGGGGTGGTTG | TATATCAAAAAATCCCCAAAAA | 62881-63243 |
| | | CT | |
| TR | GGTAGTGTAATTTGTATAAAGAGG | CACCTCATTCTAAAATTCCCAT | 169359-169564 |
| | | ATC | |
| Qp | TGTTTTTAATAGATAGAAATTGGGTG | ACCAAAATATAAAAATAACAT | 50030-50205 |
| (EBNA) | | ATATTACCC | |
| Qp | TTTGGTATTTTGGGTAGTTGGG | TATCAAAAACAAACAACAACA | 50200-50379 |
| (EBNA) | | ATCC | |
| EBNA | GGTAATTTTTGGTAGTGATTTGGATT | AAAACTTATTCCTCTTTTCCCCT | 13996-14208 |
| (intergenic) | | CT | 17068-17280 |
| | | | 20140-20352 |
| | | | 23212-23424 |
| | | | 26284-26496 |
| | | | 29356-29568 |
| | | | 32428-32640 |
| Wp | ATAAGTTTTTAGATAGGGGAGTGGG | AAAAAATAAAAAACCCCCTCTT | 14200-14441 |
| (EBNA) | | ACA | 17272-17513 |
| | | | 20344-20585 |
| | | | 23416-23657 |
| | | | 26488-26729 |
| | | | 29560-29801 |
| | | | 32632-32873 |
| BHLF1 | GTGTATTTGGAAGGTAGGGGG | CCCTAAACCCCTAAACCTATAC | 40195-40299 |
| | | CAT | |
| BVRF1 | TGATGGTTTGGAGAGTATGTAGGTT | TTACCCCTCAACTACTTAAAAA | 133411-133707 |
| | | ACA | |
| Fp | GGTTAGTTTGATTAAGGGTGAGGTTA | CCCTCAATAATCACCCAATTTC | 49898-50066 |
| | | TAT | |
| LMP1 | GGTGGTTAAGTGTAATAGGAAATGG | ATTACCCCACAACCTTACCTCA | 169285-169471 |
| | | CCTA | |
| OriP | GGGGTGTTAGAGATAATTAGTGGAGTT | AACAAAAACCAAAACAAATAA | 8413-8519 |
| | | ACCA | |
| EBNA3C | GTGATGGATGTTGGTAAGGTTTAGT | TTAAAACCTTAAAATCCAAAAA | 88234-88566 |
| | | CCC | |
| LMP2B | TTTTTAGGGAATGTTAGATTTTATTAAG | CCTTCTCTATCCACTTAAAACC | 168560-168792 |
| | | CTT | |
| EBER1 | AAGAGGGGGTTATAAAGTTTAGGGT | CAATATCTACAAATCTACATCT | 6454-6626 |
| | | CCTCAAA | |
| LMP2A | GGATTATTTTAATTGGTAAGATTTGGG | CAACAACAATATATAAAAATTA | 165834-165982 |
| | | TCACAAC | |
| BXLF1 | GTTGTGTGATTGTGTTAATTTTTTT | TTTTAACCCAAAACTAAAAACT | 132930-133073 |
| | | CTACCT | |
| BGLF4 | AGGGGGTTTTGGGGAGATATTTA | CCAAAAATCAACTACAAACAA | 111246-111429 |
| | | CTAAAA | |

Statistics: Two-tailed unpaired t-test was used unless otherwise specified. All statistical analyses were performed using Prism software (GraphPad).
Study Approval: The research and animal resource center of Weill Cornell Medical College and Memorial Sloan Kettering Cancer Center approved all murine studies.
Cell culture, immunoblot, immunohistochemistry and reagents: Kem I, Rael, and Mutu I were obtained from Wayne Tam in 2017. Kem III and Mutu III were obtained from Ben Gewurz in 2017. Daudi, Raji, and Ramos were purchased from American Type Culture Collection (ATCC) in 2013. Jiyoye was purchased from ATCC in 2014. LCL9001 was obtained by infection of peripheral blood lymphocytes with EBV. Cells were cultured in RPMI-1640 media (Invitrogen) supplemented with 10% heat inactivated fetal bovine serum and gentamicin 50 ug/mL (Sigma Aldrich). Drugs were obtained from vendors as follows: Decitabine (Selleckchem), 5-azacytidine (Selleckchem), and EPZ-6438 (Selleckchem), BEZ-235 (Selleckchem), Cladribine (Selleckchem), Cytarabine (Selleckchem), EPZ-011989 (Epizyme), EPZ-6438 (Selleckchem), Ganetespib (gifted from Leandro Cerchietti), GDC-0032 (gifted from Lewis Cantley), NSCE (Cornell Chemistry Core), Obatoclax (Selleckchem), PU-H71 (gifted from Gabriella Chiosis), Venetoclax (Selleckchem), GSK-126 (GlaxoSmithKline), doxorubicin (Selleckchem), BYL-719 (provided by Lewis Cantley). Cell viability was determined using an ATP based luminescent assay (CellTiter-Glo, Promega) and the GloMax® Multi+ detection system (Promega). IC50 values were calculated using Prism 6 software.

Immunoblot was performed with the standard procedure using the following antibodies: β-actin (GeneTex), BZLF1 (Santa Cruz), EBNA1 (Santa Cruz), EBNA2 (AbCam), EBNA3C (gift from Benjamin Gewurz), GAPDH (GeneTex), and LMP1 (AbCam).

Cell blocks were generated from cell lines in suspension by fixation in 10% formalin. Immunohistochemistry on cell blocks and mouse tumors was performed with the following antibodies: EBNA2 (abcam #ab90543), LMP1 (Dako #M0897), BZLF1 (Santa Cruz #SC-53904), CD8 (Leica #PA0183), PD-1 (Dako #M3653), PD-L1 (Cell Marque #315M-98). The Halo® image analysis software program (Indica Labs) was used to quantify immunohistochemical stains.

qRT-PCR: RNA was extracted using the Direct-zol-96 RNA kit (Zymo Research). DNase-treated total RNA was reverse transcribed with the High-Capacity cDNA Reverse Transcription Kit (Thermo Fisher Scientific). Reactions were performed in triplicate and the change in threshold cycle number (ΔCt) was calculated for each sample, normalized to a housekeeping gene (GAPDH). The ΔCt in drug treated cells was normalized to the ΔCt in vehicle treated cells to obtain ΔΔCt. Fold change in mRNA levels was calculated as 2\hat{ }(ΔΔCt).
EBV-CTLs and Cr release assay: EBV-CTLs were generated from peripheral blood mononuclear cells separated by low density separation from peripheral blood of normal consented donors by stimulation with autologous B cells transformed with B95.8 Epstein Bar Virus described in Doubrovina et al. (2012) and Roskrow et al. (1998). After 4 weeks of culture in Yssel's medium supplemented with 5% human AB serum in the presence of IL2 (50 Un/ml) and weekly re-stimulations with autologous EBV BLCLs; the T cells were characterized for their EBV specificity and HLA restriction in a standard Cr51 release assay against both autologous and a panel of EBV-positive and EBV negative targets each matching one-two HLA alleles expressed by the CTL donor. Cytotoxic activity of CTLs against each target was calculated as % of lysis=100%×(Avg CTL induced release, cpm−Avg spontaneous release (SR), cpm)/(Avg Maximum release (MR), cpm−Avg spontaneous release (SR), cpm), where the average is calculated for 3 replicates for the test wells and for 5-replicates for SR and MR wells. The HLA-A0201 restricted EBV CTLs were also characterized for the specificity to EBV antigens in Cr51 release assay against autologous EBV-negative antigen-presenting cells loaded with the A0201 EBV epitopes. The effect of hypomethylation on the susceptibility of the EBV+ Burkitt lymphoma cells (Mutu and Rael) to the EBV CTL mediated killing was tested in Cr51 assay after co-incubation of these cells with decitabine. In xenograft models EBV-CTLs were given a dose of 1-2×10⁷ T-cells/mouse. The animals were also treated with 2000 Un of Interleukin-2/mouse/dose injected i.p. twice/week.
Quantitative DNA methylation analysis using MassARRAY: DNA was isolated using the Qiagen DNeasy Blood and Tissue Kit (Qiagen) and 1 μg of DNA was converted with sodium bisulfite using the EZ DNA methylation kit (Zymo Research). DNA methylation analysis of the EBV loci was carried out using the MassARRAY EpiTYPER assay (Agena Biosciences). In brief, DNA regions of interest were amplified with PCR primers specific for the EBV gene loci (primers and genome EBV positions listed in Table 1) using the reference (B95-8 strain) genome (NCBI GenBank Accession: V01555.2). PCR products were in-vitro transcribed and fragmented with RNase A (Agena) and RNA oligonucleotide fragments were analyzed via Matrix-Assisted Laser Desorption/Ionization-Time of Flight (MALDI-ToF) mass spectrometry. Ratios of unmethylated versus methylated mass peaks were used to calculate the percentage of DNA methylation for individual CpG dinucleotides.
Agilent SureSelect MethylomeCapture (custom panel): Library preparation for methylome capture, sequencing and post-processing of the raw data was performed at the Epigenomics Core at Weill Cornell Medical College as follows: Libraries were made using SureSelect^{XT} Methyl Reagent kit (G9651B), following manufacturer's recommendations (Agilent Technologies Inc. Santa Clara, Calif.). Briefly, 1000 ng from each DNA, were sonicated using a Covaris S220 sonicator (Covaris, Woburn, Mass.) to approximately 100-175 bp fragments, end-repaired, phosphorylated, A-tailed and ligated to SureSelect methylated adaptors to create pre-capture libraries. At each step, products were cleaned by the use of Agencourt AMPure XP beads following manufacturer's recommendation (Beckman Coulter, Indianapolis, Ind.). Pre-capture libraries were hybridization to an EBV custom capture library (SureDesign ID 3189341) for 16 hrs. at 65° C. Hybridized products were recovered by purification on Dynabeads MyOne Streptavidin T1 magnetic beads, and then subjected to bisulfite conversion (64° C. for 2.5 hr) using the Zymo EZ DNA Methylation Gold kit (Cat # D5005, Zymo Research, Irvine Calif.). The post-capture bisulfite treated libraries were first PCR amplified for 8 cycles and Illumina indexes for multiplexed sequencing were added through 6 cycles of PCR amplification. Final yields were quantified in a Qubit 2.0 Fluorometer (Life Technologies, Grand Island, N.Y.), and quality of the library was assessed on a DNA1000 Bioanalyzer chip (Agilent Technologies, Santa Clara, Calif.). Libraries were normalized to 2 nM, pooled and 10% phiX added before clustering at 10 μM on a V2 pair end read flow cell and sequenced for 150 cycles on an Illumina MiSeq. Primary processing of sequencing images was done using Illumina's Real Time Analysis software (RTA) as suggested by Illumina. CASAVA 1.8.2 software was then used to demultiplex samples, generate raw reads and respective quality scores. Analysis of bisulfite treated sequence reads, was carried out as described in Garrett-Bakelman et al., except alignment was done to the EBV genome. https://www.ncbi.nlm.nih.gov/assembly/GCF 002402265.1. The percentage of bisulfite converted cytosines (representing unmethylated cytosines) and non-converted cytosines (representing methylated cytosines) were recorded for each cytosine position in CpG, CHG, and CHH contexts (with H corresponding to A, C, or T nucleotides).

### Results

High Throughput Screen Identifies Small Molecules that Induce Expression of Latency III Viral Genes in EBV+ Burkitt Lymphoma

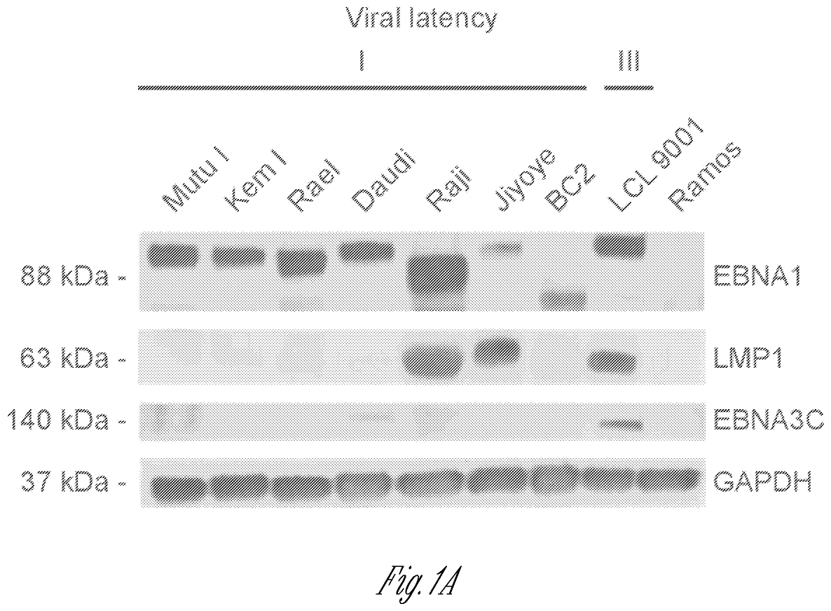

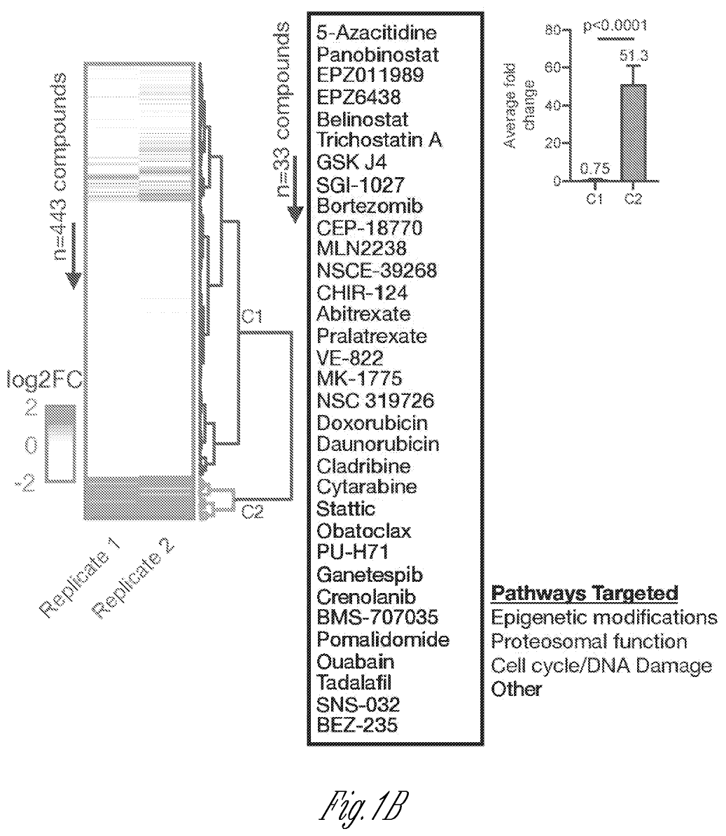

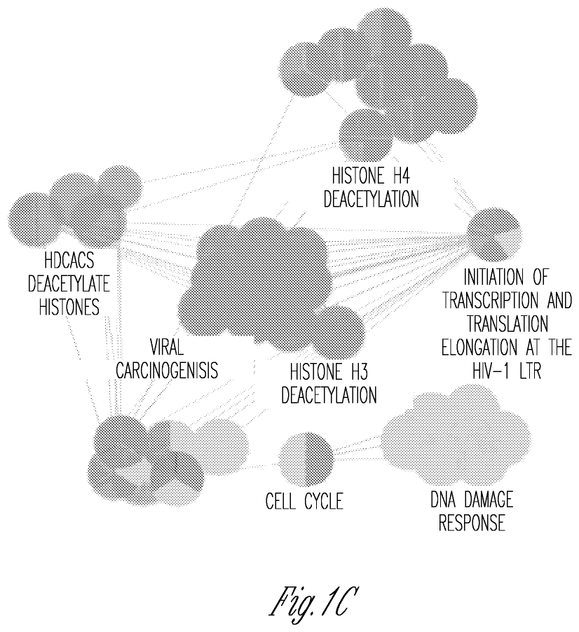

To identify small molecules that might convert a latency I EBV+ lymphoma to the latency II or III program, a high-throughput pharmacologic screen in latency I EBV+BL cells was performed. To select an appropriate cell line for the screen, a panel of EBV+BL cell lines was utilized to characterize latency. Mutu I, Kem I, Rael, Daudi, Raji, and Jiyoye BL cells were probed by immunoblot for EBNA1, LMP1, and EBNA3C. Kem I, Mutu I, and Rael expressed EBNA1 alone, indicative of latency I pattern. Raji and Jiyoye expressed high levels of LMP1 and Daudi expressed low levels of EBNA3C, likely due a latency switch in culture (FIG. 1A). Based on this, Kem I was selected for the screen and Rael and Mutu I for validation. LMP1 transcript level, as measured by qRT-PCR, was selected as the readout for the screen as this would identify cells upon conversion to latency II or latency III.

Kem I cells were incubated in 96-well format with small molecules using drug plates containing 447 validated cancer compounds (Table 2, adapted from Selleckchem Cat #L3500). This library was selected to include structurally diverse compounds covering over 200 targets including drugs targeting apoptosis, proteasome function, and epigenetic targets, as well as PIMKAKT, MAPK, JAK, and others. Cells were exposed to agents at LpM or 2.5 μM, for 48 hours. LMP1 expression was quantified by in-well qRT-PCR. The screen was performed twice, each time with technical triplicates. A compound was considered a hit if it induced a two-fold or greater change in LMP1 expression. Unsupervised clustering analysis of fold change in LMP1 revealed a group of 33 compounds inducing >2 fold change in both replicates (FIG. 1B). To assess significance a t-test was run comparing the fold changes of the non-hits (C) versus the hits (0), yielding a p-value <0.00001. Compounds on the list of hits included epigenetic modifiers, proteosome inhibitors, agents involved in modulation of cell cycle or DNA damage response as well as others (see Table 2A).

TABLE 2A

| | | Replicate 1-LMP1 | Replicate 2-LMP1 | Average LMP1 |
|---|---|---|---|---|
| Product Name | Dose (uM) | Fold Increase | Fold Increase | Fold Increase |
| (−)-MK 801 Maleate | 2.5 | 0.94 | 0.81 | 0.88 |
| (−)-Parthenolide | 2.5 | 0.26 | 0.87 | 0.57 |
| (+)-Bicuculline | 2.5 | 0.86 | 0.93 | 0.89 |
| (+)-JQ1 | 2.5 | 0.65 | 1.31 | 0.98 |
| (S)-crizotinib | 2.5 | 0.57 | 0.84 | 0.70 |
| 17-AAG (Tanespimycin) | 2.5 | 0.36 | 0.55 | 0.45 |
| 2-Methoxyestradiol | 2.5 | 0.65 | 1.34 | 1.00 |
| (2-MeOE2) | | | | |
| 4E1RCat | 2.5 | 0.30 | 1.12 | 0.71 |
| 4EGI-1 | 2.5 | 0.41 | 0.86 | 0.64 |
| 4μ8C | 2.5 | 0.19 | 7.96 | 4.08 |
| 5-Azacytidine | 1 | 5.77 | 2.43 | 4.10 |
| 5-hydroxymethyl | 2.5 | 0.49 | 0.23 | 0.36 |
| Tolterodine (PNU | | | | |
| 200577, 5-HMT, 5-HM) | | | | |
| A-769662 | 2.5 | 0.41 | 0.39 | 0.40 |
| Abitrexate | 2.5 | 3.70 | 2.64 | 3.17 |
| (Methotrexate) | | | | |
| ABT-199 (GDC-0199) | 2.5 | 0.51 | 0.98 | 0.75 |
| ABT-263 (Navitoclax) | 2.5 | 0.21 | 0.33 | 0.27 |
| Acadesine | 2.5 | 0.57 | 0.59 | 0.58 |
| ADL5859 HCl | 2.5 | 0.54 | 1.50 | 1.02 |
| ADX-47273 | 2.5 | 0.81 | 0.35 | 0.58 |
| AG-14361 | 2.5 | 0.39 | 0.22 | 0.30 |
| AGI-5198 | 2.5 | 0.71 | 0.93 | 0.82 |
| AGI-6780 | 2.5 | 0.42 | 2.60 | 1.51 |
| Agomelatine | 2.5 | 0.34 | 0.52 | 0.43 |
| Allopurinol | 2.5 | 0.09 | 0.13 | 0.11 |
| Aloxistatin | 2.5 | 0.10 | 0.12 | 0.11 |
| AM1241 | 2.5 | 1.01 | 0.56 | 0.79 |
| AMG-517 | 2.5 | 0.56 | 0.48 | 0.52 |
| Amlodipine | 2.5 | 0.51 | 3.61 | 2.06 |
| Anacetrapib (MK-0859) | 2.5 | 0.80 | 0.46 | 0.63 |
| Anastrozole | 2.5 | 0.45 | 1.42 | 0.94 |
| Aniracetam | 2.5 | 0.72 | 0.39 | 0.55 |
| AP26113 | 2.5 | 1.07 | 0.77 | 0.92 |
| Apatinib | 2.5 | 1.23 | 0.50 | 0.87 |
| Apigenin | 2.5 | 0.69 | 0.47 | 0.58 |
| Apixaban | 2.5 | 0.40 | 0.24 | 0.32 |
| Apoptosis Activator 2 | 2.5 | 0.87 | 0.78 | 0.82 |
| Aprepitant | 2.5 | 1.58 | 0.41 | 0.99 |
| AS-252424 | 2.5 | 0.57 | 0.49 | 0.53 |
| AT101 | 2.5 | 0.51 | 1.58 | 1.04 |
| Ataluren (PTC124) | 2.5 | 0.50 | 0.48 | 0.49 |
| Atglistatin | 2.5 | 0.75 | 1.23 | 0.99 |
| Atorvastatin Calcium | 2.5 | 0.05 | 0.14 | 0.09 |
| AUY922 (NVP-AUY922) | 2.5 | 1.62 | 0.96 | 1.29 |
| AVL-292 | 2.5 | 0.48 | 0.56 | 0.52 |
| AZ 3146 | 2.5 | 0.66 | 0.57 | 0.61 |
| AZ191 | 2.5 | 0.50 | 0.24 | 0.37 |
| AZ20 | 2.5 | 1.56 | 1.22 | 1.39 |
| AZD1981 | 2.5 | 1.13 | 0.46 | 0.80 |
| AZD2461 | 2.5 | 0.50 | 1.51 | 1.00 |
| AZD3463 | 2.5 | 0.28 | 0.24 | 0.26 |
| AZD4547 | 2.5 | 0.11 | 0.15 | 0.13 |
| AZD6482 | 2.5 | 0.06 | 0.37 | 0.22 |
| AZD7545 | 2.5 | 0.40 | 0.94 | 0.67 |
| AZD7762 | 2.5 | 9.74 | 1.94 | 5.84 |
| BAM7 | 2.5 | 0.51 | 1.35 | 0.93 |
| Batimastat (BB-94) | 2.5 | 0.38 | 0.55 | 0.46 |
| Bazedoxifene HCl | 2.5 | 0.52 | 0.56 | 0.54 |
| Belinostat (PXD101) | 2.5 | 17.40 | 85.87 | 51.63 |
| Bergenin | 2.5 | 0.65 | 1.07 | 0.86 |
| BEZ 235 | 1 | 17.37 | 9.2 | 13.29 |
| BGJ398 (NVP-BGJ398) | 2.5 | 1.02 | 0.69 | 0.85 |
| BI 2536 | 2.5 | 23.30 | 1.89 | 12.60 |
| BI-D1870 | 2.5 | 0.47 | 0.52 | 0.50 |
| BIBR 1532 | 2.5 | 0.63 | 0.91 | 0.77 |
| Birinapant | 2.5 | 1.05 | 1.02 | 1.03 |
| Bisoprolol fumarate | 2.5 | 0.41 | 1.73 | 1.07 |
| BML-190 | 2.5 | 1.01 | 0.53 | 0.77 |
| BMS-378806 | 2.5 | 0.75 | 0.53 | 0.64 |
| BMS-707035 | 2.5 | 18.24 | 67.95 | 43.09 |
| Bortezomib (PS-341) | 2.5 | 150.06 | 4.38 | 77.22 |
| Bosentan Hydrate | 2.5 | 1.22 | 0.40 | 0.81 |
| Bosutinib (SKI-606) | 2.5 | 0.36 | 1.26 | 0.81 |
| Brinzolamide | 2.5 | 0.23 | 0.20 | 0.21 |
| BTB06584 | 2.5 | 1.23 | 1.20 | 1.22 |
| BTZ043 Racemate | 2.5 | 0.62 | 0.67 | 0.64 |
| Bupivacaine HCl | 2.5 | 0.64 | 0.53 | 0.59 |
| BX-912 | 2.5 | 0.40 | 0.35 | 0.37 |
| C646 | 2.5 | 0.30 | 0.29 | 0.30 |
| Caffeic Acid Phenethyl | 2.5 | 0.59 | 0.33 | 0.46 |
| Ester | | | | |
| Canagliflozin | 2.5 | 0.52 | 0.75 | 0.63 |
| Candesartan | 2.5 | 0.34 | 0.52 | 0.43 |
| Captopril | 2.5 | 0.71 | 0.52 | 0.62 |
| Carvedilol | 2.5 | 0.89 | 0.87 | 0.88 |
| CCT128930 | 2.5 | 0.40 | 0.78 | 0.59 |
| Celecoxib | 2.5 | 0.63 | 0.62 | 0.62 |
| CEP-18770 | 2.5 | 98.21 | 95.63 | 96.92 |
| (Delanzomib) | | | | |
| CGK 733 | 2.5 | 0.58 | 0.84 | 0.71 |
| CGP 57380 | 2.5 | 0.81 | 1.86 | 1.33 |
| CGS 21680 HCl | 2.5 | 0.72 | 0.68 | 0.70 |
| CHIR-124 | 2.5 | 105.57 | 31.35 | 68.46 |
| CHIR-98014 | 2.5 | 0.81 | 0.54 | 0.67 |
| Cilomilast | 2.5 | 0.95 | 1.18 | 1.06 |
| Cinacalcet HCl | 2.5 | 1.17 | 0.34 | 0.75 |
| CK-636 | 2.5 | 0.69 | 1.84 | 1.27 |
| Cladribine | 1 | 71.44 | 6.02 | 38.73 |
| Clemastine Fumarate | 2.5 | 0.64 | 0.69 | 0.67 |
| CNX-774 | 2.5 | 0.51 | 0.73 | 0.62 |
| Costunolide | 2.5 | 0.79 | 1.62 | 1.21 |
| CP-673451 | 2.5 | 0.52 | 0.45 | 0.49 |
| CP-91149 | 2.5 | 0.49 | 0.80 | 0.64 |
| Crenolanib (CP-868596) | 2.5 | 2.56 | 5.53 | 4.04 |
| CRT0044876 | 2.5 | 0.55 | 0.33 | 0.44 |
| Cryptotanshinone | 2.5 | 0.94 | 0.77 | 0.85 |
| Cyproterone Acetate | 2.5 | 0.27 | 0.24 | 0.26 |
| Cytarbine | 1 | 2.61 | 2.01 | 2.31 |
| Dabrafenib | 2.5 | 0.61 | 1.22 | 0.92 |
| (GSK2118436) | | | | |
| Dalcetrapib (JTT-705, | 2.5 | 0.61 | 1.34 | 0.98 |
| RO4607381) | | | | |
| Dapagliflozin | 2.5 | 0.69 | 1.34 | 1.01 |
| Daunorubicin HCl | 2.5 | 220.95 | 262.46 | 241.71 |
| DBeQ | 2.5 | 0.72 | 1.25 | 0.99 |
| DCC-2036 (Rebastinib) | 2.5 | 0.50 | 0.25 | 0.38 |
| DMH1 | 2.5 | 0.21 | 1.28 | 0.75 |
| DMXAA (Vadimezan) | 2.5 | 0.44 | 1.29 | 0.87 |
| Doxazosin Mesylate | 2.5 | 0.67 | 0.95 | 0.81 |
| Doxorubicin | 1 | 39.18 | 54.78 | 46.98 |
| Dutasteride | 2.5 | 0.38 | 0.41 | 0.39 |
| Dynasore | 2.5 | 0.64 | 0.89 | 0.77 |
| E-64 | 2.5 | 0.48 | 0.63 | 0.55 |
| EHop-016 | 2.5 | 0.70 | 1.43 | 1.06 |
| Elvitegravir (GS-9137, | 2.5 | 1.25 | 1.20 | 1.23 |
| JTK-303) | | | | |
| Embelin | 2.5 | 0.43 | 0.74 | 0.59 |
| Empagliflozin (BI 10773) | 2.5 | 0.67 | 0.50 | 0.58 |
| Enalaprilat Dihydrate | 2.5 | 0.51 | 0.52 | 0.51 |
| Entacapone | 2.5 | 0.49 | 0.71 | 0.60 |
| Enzalutamide | 2.5 | 0.88 | 1.05 | 0.96 |
| (MDV3100) | | | | |
| Enzastaurin (LY317615) | 2.5 | 0.48 | 49.80 | 25.14 |
| EPZ 011989 | 1 | 4.1 | 56.88 | 30.49 |
| EPZ 6438 | 1 | 2.14 | 46.85 | 24.50 |
| Erastin | 2.5 | 0.70 | 0.75 | 0.73 |
| Esomeprazole Sodium | 2.5 | 0.16 | 0.44 | 0.30 |
| Etodolac | 2.5 | 0.57 | 1.10 | 0.84 |
| Etomidate | 2.5 | 0.56 | 0.44 | 0.50 |
| EUK 134 | 2.5 | 0.85 | 0.95 | 0.90 |
| Everolimus (RAD001) | 2.5 | 2.00 | 1.31 | 1.65 |
| EX 527 (Selisistat) | 2.5 | 0.24 | 1.38 | 0.81 |
| Exemestane | 2.5 | 0.53 | 0.86 | 0.70 |
| Felodipine | 2.5 | 0.84 | 0.69 | 0.77 |
| Ferrostatin-1 (Fer-1) | 2.5 | 0.24 | 0.19 | 0.22 |
| FG-4592 | 2.5 | 0.19 | 0.24 | 0.22 |
| Finasteride | 2.5 | 0.51 | 0.81 | 0.66 |
| Fingolimod (FTY720) | 2.5 | 0.33 | 1.06 | 0.69 |
| HCl | | | | |
| FLI-06 | 2.5 | 0.19 | 0.73 | 0.46 |
| Fluvastatin Sodium | 2.5 | 0.84 | 1.04 | 0.94 |
| Fluvoxamine maleate | 2.5 | 0.97 | 0.37 | 0.67 |
| Formoterol | 2.5 | 1.71 | 1.96 | 1.83 |
| Hemifumarate | | | | |
| Forskolin | 2.5 | 1.39 | 1.11 | 1.25 |
| Fostamatinib (R788) | 2.5 | 0.24 | 1.11 | 0.67 |
| Fulvestrant | 2.5 | 0.16 | 0.18 | 0.17 |
| Ganetespib | 1 | 8.07 | 38.8 | 23.44 |
| GDC-0032 | 1 | 1.86 | 0.59 | 1.23 |
| GDC-0068 | 2.5 | 0.27 | 0.27 | 0.27 |
| GDC-0152 | 2.5 | 0.76 | 0.59 | 0.68 |
| GDC-0941 | 2.5 | 0.75 | 1.09 | 0.92 |
| Ginkgolide A | 2.5 | 1.33 | 0.49 | 0.91 |
| Ginkgolide B | 2.5 | 1.04 | 0.49 | 0.76 |
| Gliclazide | 2.5 | 0.98 | 0.49 | 0.73 |
| Gliquidone | 2.5 | 0.81 | 0.73 | 0.77 |
| GNF-2 | 2.5 | 0.21 | 1.91 | 1.06 |
| Go 6983 | 2.5 | 0.22 | 0.45 | 0.34 |
| Golgicide A | 2.5 | 0.34 | 0.19 | 0.27 |
| Granisetron HCl | 2.5 | 0.25 | 0.24 | 0.25 |
| GSK J4 HCl | 2.5 | 48.53 | 16.15 | 32.34 |
| GSK1292263 | 2.5 | 0.20 | 0.34 | 0.27 |
| GSK1904529A | 2.5 | 0.18 | 0.23 | 0.20 |
| GSK2656157 | 2.5 | 0.58 | 0.84 | 0.71 |
| GSK429286A | 2.5 | 0.60 | 0.54 | 0.57 |
| GSK461364 | 2.5 | 1.73 | 1.50 | 1.62 |
| GSK690693 | 2.5 | 0.85 | 0.41 | 0.63 |
| GW0742 | 2.5 | 0.53 | 0.99 | 0.76 |
| GW2580 | 2.5 | 0.50 | 0.48 | 0.49 |
| GW3965 HCl | 2.5 | 0.20 | 0.35 | 0.27 |
| GW4064 | 2.5 | 0.94 | 0.50 | 0.72 |
| GW441756 | 2.5 | 0.70 | 0.89 | 0.80 |
| GW9508 | 2.5 | 0.52 | 0.45 | 0.48 |
| GW9662 | 2.5 | 0.26 | 0.89 | 0.58 |
| H 89 2HCl | 2.5 | 0.54 | 0.82 | 0.68 |
| HA14-1 | 2.5 | 0.62 | 0.83 | 0.73 |
| HC-030031 | 2.5 | 0.32 | 0.24 | 0.28 |
| I-BET151 | 2.5 | 1.41 | 1.38 | 1.39 |
| (GSK1210151A) | | | | |
| Ibrutinib (PCl-32765) | 2.5 | 0.09 | 0.22 | 0.15 |
| ICG-001 | 2.5 | 1.17 | 0.92 | 1.04 |
| Icotinib | 2.5 | 15.74 | 0.34 | 8.04 |
| Ifenprodil Tartrate | 2.5 | 0.58 | 1.35 | 0.96 |
| IKK-16 (IKK Inhibitor VII) | 2.5 | 0.26 | 0.35 | 0.30 |
| Ilomastat (GM6001, | 2.5 | 0.05 | 0.12 | 0.08 |
| Galardin) | | | | |
| Imatinib (STI571) | 2.5 | 0.60 | 0.25 | 0.42 |
| IMD 0354 | 2.5 | 0.74 | 0.50 | 0.62 |
| Imidapril HCl | 2.5 | 0.88 | 0.44 | 0.66 |
| Iniparib (BSl-201) | 2.5 | 0.51 | 0.76 | 0.63 |
| IOX2 | 2.5 | 0.40 | 0.38 | 0.39 |
| IPA-3 | 2.5 | 0.21 | 0.33 | 0.27 |
| Irinotecan | 2.5 | 0.51 | 0.68 | 0.59 |
| Irinotecan HCl | 2.5 | 0.69 | 2.06 | 1.38 |
| Trihydrate | | | | |
| Isotretinoin | 2.5 | 1.42 | 2.10 | 1.76 |
| Ispinesib (SB-715992) | 2.5 | 2.72 | 0.43 | 1.57 |
| Istradefylline | 2.5 | 0.35 | 1.06 | 0.70 |
| Ivacaftor (VX-770) | 2.5 | 2.22 | 0.49 | 1.35 |
| JNJ-1661010 | 2.5 | 0.31 | 0.70 | 0.51 |
| JSH-23 | 2.5 | 0.86 | 0.69 | 0.77 |
| Ki16198 | 2.5 | 1.53 | 1.12 | 1.33 |
| Ki16425 | 2.5 | 0.80 | 0.45 | 0.62 |
| KPT-185 | 2.5 | 0.56 | 0.80 | 0.68 |
| KPT-276 | 2.5 | 0.97 | 2.91 | 1.94 |
| KPT-330 | 2.5 | 0.28 | 0.42 | 0.35 |
| KU-55933 (ATM Kinase | 2.5 | 0.60 | 0.95 | 0.78 |
| Inhibitor) | | | | |
| KU-60019 | 2.5 | 0.49 | 0.53 | 0.51 |
| KX2-391 | 2.5 | 2.01 | 1.25 | 1.63 |
| Lafutidine | 2.5 | 0.94 | 0.76 | 0.85 |
| LB42708 | 2.5 | 0.46 | 0.16 | 0.31 |
| LDE225 (NVP- | 2.5 | 0.50 | 0.29 | 0.39 |
| LDE225, Erismodegib) | | | | |
| LDK378 | 2.5 | 0.85 | 0.49 | 0.67 |
| LDN-212854 | 2.5 | 0.96 | 0.27 | 0.62 |
| Lenalidomide (CC-5013) | 2.5 | 1.57 | 1.91 | 1.74 |
| Letrozole | 2.5 | 0.66 | 1.44 | 1.05 |
| Levosulpiride | 2.5 | 0.70 | 0.30 | 0.50 |
| Linagliptin | 2.5 | 1.01 | 0.81 | 0.91 |
| Lomeguatrib | 2.5 | 0.57 | 1.61 | 1.09 |
| Loratadine | 2.5 | 0.24 | 0.32 | 0.28 |
| Losartan Potassium | 2.5 | 0.21 | 0.48 | 0.35 |
| (DuP 753) | | | | |
| Lovastatin | 2.5 | 0.34 | 1.59 | 0.96 |
| Loxistatin Acid (E-64C) | 2.5 | 0.62 | 0.48 | 0.55 |
| LY2157299 | 2.5 | 0.30 | 0.30 | 0.30 |
| LY2228820 | 2.5 | 0.63 | 0.56 | 0.60 |
| LY2603618 | 2.5 | 0.94 | 1.47 | 1.21 |
| LY2784544 | 2.5 | 1.24 | 0.65 | 0.94 |
| LY411575 | 2.5 | 0.41 | 0.31 | 0.36 |
| Maraviroc | 2.5 | 0.44 | 1.72 | 1.08 |
| Mdivi-1 | 2.5 | 0.51 | 0.77 | 0.64 |
| Memantine HCl | 2.5 | 0.55 | 0.30 | 0.42 |
| Mirabegron | 2.5 | 0.74 | 0.76 | 0.75 |
| MK-1775 | 2.5 | 6.01 | 4.43 | 5.22 |
| MK-2206 2HCl | 2.5 | 0.44 | 0.59 | 0.51 |
| MK-2866 (GTx-024) | 2.5 | 0.72 | 0.56 | 0.64 |
| MK-8245 | 2.5 | 1.51 | 0.84 | 1.17 |
| ML130 (Nodinitib-1) | 2.5 | 0.43 | 1.19 | 0.81 |
| ML133 HCl | 2.5 | 0.42 | 0.67 | 0.54 |
| ML161 | 2.5 | 1.11 | 0.54 | 0.83 |
| ML347 | 2.5 | 0.19 | 0.49 | 0.34 |
| MLN2238 | 2.5 | 86.75 | 137.73 | 112.24 |
| MLN8054 | 2.5 | 0.27 | 0.47 | 0.37 |
| MM-102 | 2.5 | 0.27 | 0.27 | 0.27 |
| MNS (3,4- | 2.5 | 0.54 | 1.68 | 1.11 |
| Methylenedioxy-β- | | | | |
| nitrostyrene, MDBN) | | | | |
| Moclobemide | 2.5 | 0.28 | 0.81 | 0.55 |
| (Ro 111163) | | | | |
| Mozavaptan | 2.5 | 0.32 | 0.90 | 0.61 |
| MRS 2578 | 2.5 | 0.64 | 0.31 | 0.47 |
| Mubritinib (TAK 165) | 2.5 | 0.74 | 0.62 | 0.68 |
| Naftopidil | 2.5 | 1.36 | 0.95 | 1.15 |
| Naltrexone HCl | 2.5 | 0.34 | 0.19 | 0.27 |
| Naproxen | 2.5 | 0.69 | 0.56 | 0.62 |
| Nebivolol | 2.5 | 0.83 | 1.49 | 1.16 |
| Necrostatin-1 | 2.5 | 0.99 | 1.17 | 1.08 |
| NH125 | 2.5 | 0.94 | 0.46 | 0.70 |
| Nilotinib (AMN-107) | 2.5 | 0.20 | 0.33 | 0.27 |
| Nilvadipine | 2.5 | 0.96 | 0.47 | 0.72 |
| NLG919 | 2.5 | 0.79 | 0.49 | 0.64 |
| NMS-873 | 2.5 | 1.05 | 0.95 | 1.00 |
| NPS-2143 | 2.5 | 0.87 | 0.62 | 0.74 |
| NSC 23766 | 2.5 | 0.62 | 1.01 | 0.82 |
| NSC 319726 | 2.5 | 7.26 | 32.48 | 19.87 |
| NSC 405020 | 2.5 | 0.50 | 0.28 | 0.39 |
| NSC697923 | 2.5 | 0.10 | 0.09 | 0.09 |
| NSCE-39268 | 1 | 3.16 | 2.19 | 2.68 |
| NU7026 | 2.5 | 0.47 | 1.07 | 0.77 |
| NVP-ADW742 | 2.5 | 0.27 | 0.53 | 0.40 |
| Obatoclax | 1 | 24.31 | 3.03 | 13.67 |
| OC000459 | 2.5 | 0.64 | 0.48 | 0.56 |
| Odanacatib (MK-0822) | 2.5 | 0.58 | 0.54 | 0.56 |
| OG-L002 | 2.5 | 1.03 | 1.27 | 1.15 |
| Oligomycin A | 2.5 | 1.03 | 1.76 | 1.40 |
| Org 27569 | 2.5 | 0.49 | 0.53 | 0.51 |
| OSI-420 | 2.5 | 0.84 | 0.49 | 0.66 |
| OSI-906 (Linsitinib) | 2.5 | 0.68 | 0.94 | 0.81 |
| OSU-03012 (AR-12) | 2.5 | 0.79 | 1.12 | 0.95 |
| OTX015 | 2.5 | 0.14 | 1.12 | 0.63 |
| Ouabain | 2.5 | 22.82 | 161.38 | 92.10 |
| Oxcarbazepine | 2.5 | 0.54 | 0.38 | 0.46 |
| Oxymetazoline HCl | 2.5 | 0.60 | 0.72 | 0.66 |
| Ozagrel | 2.5 | 1.27 | 1.55 | 1.41 |
| Ozagrel HCl | 2.5 | 0.85 | 0.72 | 0.79 |
| P22077 | 2.5 | 0.20 | 0.29 | 0.25 |
| PAC-1 | 2.5 | 0.78 | 0.78 | 0.78 |
| Pacritinib (SB1518) | 2.5 | 0.24 | 0.49 | 0.37 |
| Palbociclib (PD- | 2.5 | 0.77 | 1.91 | 1.34 |
| 0332991) HCl | | | | |
| Pancuronium dibromide | 2.5 | 0.25 | 0.31 | 0.28 |
| Panobinostat (LBH589) | 2.5 | 451.65 | 314.88 | 383.26 |
| PD0325901 | 2.5 | 0.68 | 0.27 | 0.47 |
| PD128907 HCl | 2.5 | 0.69 | 1.20 | 0.94 |
| PD184352 (CI-1040) | 2.5 | 0.65 | 0.66 | 0.66 |
| PF-04217903 | 2.5 | 0.96 | 1.11 | 1.03 |
| PF-3845 | 2.5 | 0.16 | 0.48 | 0.32 |
| PF-4708671 | 2.5 | 1.30 | 0.18 | 0.74 |
| PF-5274857 | 2.5 | 1.12 | 0.62 | 0.87 |
| PF-562271 | 2.5 | 0.81 | 0.19 | 0.50 |
| PF-573228 | 2.5 | 0.87 | 0.45 | 0.66 |
| PFI-1 (PF-6405761) | 2.5 | 0.42 | 1.15 | 0.78 |
| PHA-665752 | 2.5 | 0.55 | 0.84 | 0.69 |
| PHA-793887 | 2.5 | 0.58 | 5.05 | 2.82 |
| Piceatannol | 2.5 | 0.83 | 0.66 | 0.75 |
| Pifithrin-μ | 2.5 | 0.40 | 1.17 | 0.78 |
| Pimobendan | 2.5 | 1.11 | 0.64 | 0.87 |
| PluriSln #1 (NSC 14613) | 2.5 | 1.70 | 0.94 | 1.32 |
| PNU-120596 | 2.5 | 0.17 | 0.44 | 0.30 |
| Pomalidomide | 2.5 | 3.72 | 7.87 | 5.80 |
| PP2 | 2.5 | 0.73 | 1.28 | 1.01 |
| PR-619 | 2.5 | 0.23 | 0.39 | 0.31 |
| Pralatrexate | 2.5 | 3.95 | 3.02 | 3.49 |
| Pramipexole | 2.5 | 0.60 | 1.27 | 0.93 |
| Propranolol HCl | 2.5 | 0.60 | 0.72 | 0.66 |
| PRT062607(P505-15, | 2.5 | 0.25 | 0.83 | 0.54 |
| BIIB057) HCl | | | | |
| PTC-209 | 2.5 | 0.34 | 1.46 | 0.90 |
| PU-H71 | 1 | 15.27 | 2.79 | 9.03 |
| PYR-41 | 2.5 | 0.50 | 0.49 | 0.49 |
| Pyrimethamine | 2.5 | 0.70 | 0.90 | 0.80 |
| Quizartinib (AC220) | 2.5 | 0.71 | 1.15 | 0.93 |
| Raltegravir (MK-0518) | 2.5 | 0.59 | 2.02 | 1.31 |
| Ramelteon | 2.5 | 0.45 | 0.22 | 0.33 |
| Ranitidine | 2.5 | 0.90 | 1.18 | 1.04 |
| Rasagiline Mesylate | 2.5 | 0.86 | 0.51 | 0.69 |
| Rebamipide | 2.5 | 0.48 | 1.23 | 0.85 |
| RepSox | 2.5 | 0.96 | 0.33 | 0.64 |
| Ridaforolimus | 2.5 | 6.08 | 0.67 | 3.37 |
| (Deforolimus, MK-8669) | | | | |
| Rigosertib (ON-01910) | 2.5 | 0.69 | 0.46 | 0.57 |
| Rimonabant | 2.5 | 0.24 | 0.54 | 0.39 |
| Rivaroxaban | 2.5 | 0.08 | 0.20 | 0.14 |
| Rizatriptan Benzoate | 2.5 | 0.10 | 0.20 | 0.15 |
| RKI-1447 | 2.5 | 0.75 | 0.33 | 0.54 |
| Rolipram | 2.5 | 1.13 | 0.75 | 0.94 |
| Rotundine | 2.5 | 0.30 | 0.27 | 0.28 |
| Roxatidine Acetate HCl | 2.5 | 0.15 | 0.24 | 0.20 |
| Ruxolitinib | 2.5 | 0.48 | 0.34 | 0.41 |
| (INCB018424) | | | | |
| S3I-201 | 2.5 | 0.63 | 0.67 | 0.65 |
| Safinamide Mesylate | 2.5 | 0.20 | 1.71 | 0.95 |
| Sal003 | 2.5 | 0.76 | 0.53 | 0.64 |
| SANT-1 | 2.5 | 0.31 | 0.97 | 0.64 |
| SAR131675 | 2.5 | 0.30 | 0.49 | 0.40 |
| SB203580 | 2.5 | 0.54 | 0.59 | 0.57 |
| SB408124 | 2.5 | 0.81 | 0.53 | 0.67 |
| SB415286 | 2.5 | 0.59 | 0.84 | 0.71 |
| SB431542 | 2.5 | 0.82 | 0.61 | 0.71 |
| SB705498 | 2.5 | 0.38 | 0.48 | 0.43 |
| SB742457 | 2.5 | 1.09 | 0.55 | 0.82 |
| SB743921 | 2.5 | 1.10 | 2.41 | 1.76 |
| SC-514 | 2.5 | 0.61 | 1.49 | 1.05 |
| SC144 | 2.5 | 1.00 | 2.02 | 1.51 |
| Selumetinib (AZD6244) | 2.5 | 0.64 | 0.44 | 0.54 |
| Semagacestat | 2.5 | 0.31 | 0.59 | 0.45 |
| (LY450139) | | | | |
| Sertraline HCl | 2.5 | 0.81 | 1.33 | 1.07 |
| SGC 0946 | 2.5 | 0.26 | 0.31 | 0.29 |
| SGI-1027 | 2.5 | 13.12 | 93.95 | 53.53 |
| SGI-1776 free base | 2.5 | 1.94 | 0.57 | 1.26 |
| Sirtinol | 2.5 | 0.28 | 0.92 | 0.60 |
| Sitaxentan sodium | 2.5 | 0.16 | 0.34 | 0.25 |
| SKI II | 2.5 | 0.96 | 1.03 | 1.00 |
| SMI-4a | 2.5 | 0.29 | 0.53 | 0.41 |
| SN-38 | 2.5 | 0.95 | 1.26 | 1.10 |
| SNS-032 (BMS-387032) | 2.5 | 23.56 | 24.39 | 23.97 |
| SNS-314 Mesylate | 2.5 | 0.71 | 0.32 | 0.51 |
| Sodium 4- | 2.5 | 0.19 | 0.53 | 0.36 |
| Aminosalicylate | | | | |
| Sorafenib | 2.5 | 0.43 | 0.64 | 0.54 |
| Sotrastaurin | 2.5 | 0.30 | 0.31 | 0.31 |
| SP600125 | 2.5 | 0.67 | 0.53 | 0.60 |
| SRPIN340 | 2.5 | 1.24 | 0.44 | 0.84 |
| SRT1720 | 2.5 | 0.58 | 0.70 | 0.64 |
| SSR128129E | 2.5 | 0.36 | 0.52 | 0.44 |
| Stattic | 2.5 | 50.36 | 2.79 | 26.57 |
| Stavudine (d4T) | 2.5 | 0.83 | 0.63 | 0.73 |
| STF-118804 | 2.5 | 0.08 | 0.11 | 0.10 |
| SU11274 | 2.5 | 1.05 | 0.82 | 0.94 |
| Suvorexant (MK-4305) | 2.5 | 0.37 | 2.57 | 1.47 |
| T0070907 | 2.5 | 0.63 | 0.50 | 0.56 |
| T0901317 | 2.5 | 0.41 | 0.47 | 0.44 |
| Tadalafil | 2.5 | 3.80 | 2.32 | 3.06 |
| TAE226 (NVP-TAE226) | 2.5 | 1.38 | 1.31 | 1.35 |
| TAK-700 (Orteronel) | 2.5 | 0.75 | 1.06 | 0.90 |
| TAK-875 | 2.5 | 0.55 | 0.59 | 0.57 |
| Tandutinib (MLN518) | 2.5 | 0.21 | 0.41 | 0.31 |
| Tariquidar | 2.5 | 0.56 | 0.87 | 0.71 |
| TCID | 2.5 | 0.66 | 0.38 | 0.52 |
| TCS 359 | 2.5 | 0.62 | 0.19 | 0.40 |
| Telmisartan | 2.5 | 0.29 | 0.45 | 0.37 |
| Temsirolimus (CCI-779, | 2.5 | 1.06 | 310.48 | 155.77 |
| NSC 683864) | | | | |
| Tenofovir | 2.5 | 0.18 | 0.19 | 0.18 |
| Tenofovir Disoproxil | 2.5 | 0.21 | 0.90 | 0.56 |
| Fumarate | | | | |
| Tenovin-6 | 2.5 | 1.55 | 2.03 | 1.79 |
| TG100-115 | 2.5 | 0.33 | 0.73 | 0.53 |
| Thiazovivin | 2.5 | 1.05 | 1.04 | 1.05 |
| Ticagrelor | 2.5 | 0.61 | 0.70 | 0.66 |
| Ticlopidine HCl | 2.5 | 0.52 | 1.78 | 1.15 |
| Tie2 kinase inhibitor | 2.5 | 0.36 | 1.81 | 1.08 |
| Tioxolone | 2.5 | 0.70 | 0.41 | 0.55 |
| Tofacitinib (CP- | 2.5 | 0.09 | 0.82 | 0.46 |
| 690550,Tasocitinib) | | | | |
| Tolazoline HCl | 2.5 | 0.26 | 0.27 | 0.26 |
| Tolfenamic Acid | 2.5 | 0.11 | 0.20 | 0.15 |
| Tolvaptan | 2.5 | 1.11 | 8.24 | 4.67 |
| Torcetrapib | 2.5 | 0.40 | 0.60 | 0.50 |
| Toremifene Citrate | 2.5 | 0.55 | 0.54 | 0.54 |
| Tosedostat (CHR2797) | 2.5 | 0.56 | 0.72 | 0.64 |
| TPCA-1 | 2.5 | 0.59 | 0.36 | 0.48 |
| Tranylcypromine | 2.5 | 0.22 | 0.45 | 0.33 |
| (2-PCPA) HCl | | | | |
| Trelagliptin | 2.5 | 0.96 | 0.31 | 0.63 |
| Triamterene | 2.5 | 0.56 | 1.26 | 0.91 |
| Trichostatin A (TSA) | 2.5 | 44.18 | 132.40 | 88.29 |
| Trimebutine | 2.5 | 2.11 | 0.63 | 1.37 |
| Tropicamide | 2.5 | 0.73 | 1.40 | 1.06 |
| Trospium chloride | 2.5 | 0.33 | 0.37 | 0.35 |
| TWS119 | 2.5 | 0.62 | 0.49 | 0.56 |
| Tyrphostin AG 879 | 2.5 | 0.53 | 0.95 | 0.74 |
| U-104 | 2.5 | 0.47 | 0.59 | 0.53 |
| U0126-EtOH | 2.5 | 0.51 | 0.65 | 0.58 |
| UNC2250 | 2.5 | 0.54 | 0.29 | 0.41 |
| UNC669 | 2.5 | 0.75 | 0.25 | 0.50 |
| URB597 | 2.5 | 0.52 | 0.55 | 0.54 |
| Vandetanib (ZD6474) | 2.5 | 0.81 | 1.47 | 1.14 |
| Varespladib (LY315920) | 2.5 | 0.35 | 0.69 | 0.52 |
| VE-821 | 2.5 | 0.20 | 0.14 | 0.17 |
| VE-822 | 2.5 | 3.28 | 4.78 | 4.03 |
| Veliparib (ABT-888) | 2.5 | 0.71 | 1.45 | 1.08 |
| Vemurafenib (PLX4032, | 2.5 | 0.33 | 0.50 | 0.41 |
| RG7204) | | | | |
| Vildagliptin (LAF-237) | 2.5 | 0.58 | 0.87 | 0.72 |
| Voriconazole | 2.5 | 1.02 | 1.94 | 1.48 |
| VU 0357121 | 2.5 | 0.51 | 0.46 | 0.48 |
| VU 0364439 | 2.5 | 1.34 | 0.45 | 0.90 |
| VU 0364770 | 2.5 | 0.51 | 1.55 | 1.03 |
| VX-680 (Tozasertib, | 2.5 | 0.96 | 0.52 | 0.74 |
| MK-0457) | | | | |
| VX-745 | 2.5 | 0.75 | 0.24 | 0.49 |
| VX-765 | 2.5 | 0.45 | 0.37 | 0.41 |
| VX-809 (Lumacaftor) | 2.5 | 0.55 | 1.18 | 0.87 |
| Wnt-C59 (C59) | 2.5 | 0.60 | 1.35 | 0.98 |
| WZ4002 | 2.5 | 0.58 | 1.23 | 0.90 |
| WZ4003 | 2.5 | 0.79 | 0.64 | 0.72 |
| WZ811 | 2.5 | 0.08 | 0.08 | 0.08 |
| XAV-939 | 2.5 | 0.73 | 0.73 | 0.73 |
| XL335 | 2.5 | 0.39 | 0.14 | 0.27 |
| YM155 (Sepantronium | 2.5 | 0.00 | 0.00 | 0.00 |
| Bromide) | | | | |
| YO-01027 | 2.5 | 0.44 | 0.27 | 0.35 |
| ZCL278 | 2.5 | 1.26 | 0.44 | 0.85 |
| Zebularine | 2.5 | 0.24 | 0.79 | 0.52 |
| Zibotentan (ZD4054) | 2.5 | 0.57 | 0.97 | 0.77 |
| ZM 306416 | 2.5 | 0.70 | 1.00 | 0.85 |
| ZM 447439 | 2.5 | 0.34 | 0.40 | 0.37 |
| Zosuquidar (LY335979) | 2.5 | 0.64 | 0.94 | 0.79 |
| 3HCl | | | | |

TABLE 2B
Targets and Pathways of the Agents in Table 2A.

| Target(s) | Pathway |
|---|---|
| GluR | Protein Tyrosine Kinase |
| E3 Ligase | PI3K/Akt/mTOR |
| GABA Receptor | Cytoskeletal Signaling |
| Epigenetic Reader Domain | Metabolism |
| MTH | Protein Tyrosine Kinase |
| HSP (e.g. HSP90) | Proteases |
| HIF | Cell Cycle/DNA Damage |
| ELF4 | Neuronal Signaling |
| ELF4 | Metabolism |
| Others | GPCR & G Protein |
| DNMT1 methyltransferase | Epigenetics |
| AChR | PI3K/Akt/mTOR |
| AMPK | MAPK |
| DHFR | Cell Cycle/DNA Damage |
| Bcl-2 | Apoptosis |
| Bcl-2 | Apoptosis |
| AMPK | Cell Cycle/DNA Damage |
| Opioid Receptor | Others |
| GluR | Protein Tyrosine Kinase |
| PARP | JAK/STAT |
| Dehydrogenase | Angiogenesis |
| IDH2 | Apoptosis |
| 5-HT Receptor | MAPK |
| OX Receptor | Metabolism |
| Cysteine protease | GPCR & G Protein |
| Cannabinoid Receptor | Neuronal Signaling |
| TRPV | Transmembrane Transporters |
| Calcium Channel | GPCR & G Protein |
| CETP | Cytoskeletal Signaling |
| Aromatase | Others |
| AMPA Receptor-kainate | Protein Tyrosine Kinase |
| Receptor-NMDA Receptor | |
| ALK | Protein Tyrosine Kinase |
| VEGFR | Protein Tyrosine Kinase |
| P450 (e.g. CYP17) | Neuronal Signaling |
| Factor Xa | Endocrinology & Hormones |
| Caspase | Microbiology |
| Substance P | Proteases |
| PI3K | Epigenetics |
| Bcl-2 | Endocrinology & Hormones |
| CFTR | MAPK |
| ATGL | Apoptosis |
| HMG-CoA Reductase | Angiogenesis |
| HSP (e.g. HSP90) | Proteases |
| BTK | GPCR & G Protein |
| Kinesin | Protein Tyrosine Kinase |
| Others | GPCR & G Protein |
| ATM/ATR | Transmembrane Transporters |
| GPR | Ubiquitin |
| PARP | Apoptosis |
| ALK | Cell Cycle/DNA Damage |
| FGFR | Endocrinology & Hormones |
| PI3K | Metabolism |
| PDHK | Epigenetics |
| Chk | PI3K/Akt/mTOR |
| Bcl-2 | Metabolism |
| MMP | Metabolism |
| Estrogen/progestogen Receptor | Neuronal Signaling |
| HDAC | Epigenetics |
| Others | Apoptosis |
| p110α/γ/β/δ, mTOR | PI3K/Akt/mTOR |
| FGFR | Neuronal Signaling |
| PLK | Ubiquitin |
| S6 Kinase | Others |
| Telomerase | Ubiquitin |
| IAP | Protein Tyrosine Kinase |
| Adrenergic Receptor | Epigenetics |
| Cannabinoid | Receptor Others |
| gp120/CD4 | Transmembrane Transporters |
| Integrase | Others |
| Proteasome | Proteases |
| Endothelin Receptor | Microbiology |
| Src | Others |
| Carbonic Anhydrase | Neuronal Signaling |
| Fo-ATPase | Angiogenesis |
| Others | GPCR & G Protein |
| cAMP | Neuronal Signaling |
| PDK-1 | Stem Cells & Wnt |
| Histone Acetyltransferase | Endocrinology & Hormones |
| NF-κB | TGF-beta/Smad |
| SGLT | Others |
| RAAS | Angiogenesis |
| RAAS | Cell Cycle/DNA Damage |
| Adrenergic Receptor | TGF-beta/Smad |
| Akt | Epigenetics |
| COX | Transmembrane Transporters |
| Proteasome | Proteases |
| ATM/ATR | Cell Cycle/DNA Damage |
| MNK | NF-κB |
| Adenosine A2 | Angiogenesis |
| Chk | Cell Cycle/DNA Damage |
| GSK-3 | Neuronal Signaling |
| PDE | Apoptosis |
| CaSR | JAK/STAT |
| Arp2/3 | Epigenetics |
| Adenosine Deaminase | Cell Cycle/DNA Damage |
| Histamine Receptor | GPCR & G Protein |
| BTK | GPCR & G Protein |
| Telomerase | Others |
| PDGFR | Epigenetics |
| Phosphorylase | Apoptosis |
| PDGFR | Others |
| APE | PI3K/Akt/mTOR |
| STAT | Proteases |
| Androgen Receptor | Metabolism |
| DNA synthesis | Cell Cycle/DNA Damage |
| Raf | Metabolism |
| CETP | Metabolism |
| SGLT | Cell Cycle/DNA Damage |
| Toposiomerase II | Cell Cycle/DNA Damage |
| p97 | GPCR & G Protein |
| Bcr-Abl | Cell Cycle/DNA Damage |
| BMP | Angiogenesis |
| VDA | Neuronal Signaling |
| Adrenergic Receptor | Protein Tyrosine Kinase |
| DNA topoisomerase II | Cell Cycle/DNA Damage |
| 5-alpha Reductase | Cell Cycle/DNA Damage |
| Dynamin | Endocrinology & Hormones |
| Cathepsin K | Microbiology |
| Rac | Epigenetics |
| Integrase | Stem Cells & Wnt |
| IAP | Cytoskeletal Signaling |
| SGLT | PI3K/Akt/mTOR |
| RAAS | GPCR & G Protein |
| Histone Methyltransferase | PI3K/Akt/mTOR |
| Androgen Receptor | Cell Cycle/DNA Damage |
| PKC | Epigenetics |
| EZH2 | Epigenetics |
| EZH2 | Epigenetics |
| Ferroptosis | Metabolism |
| ATPase | Metabolism |
| COX | Transmembrane Transporters |
| GABA Receptor | JAK/STAT |
| Beta Amyloid | Metabolism |
| mTOR | Protein Tyrosine Kinase |
| Sirtuin | Others |
| Aromatase | Cell Cycle/DNA Damage |
| Calcium Channel | Neuronal Signaling |
| Ferroptosis | Neuronal Signaling |
| HIF | Angiogenesis |
| 5-alpha Reductase | Apoptosis |
| S1P Receptor | Others |
| Notch | Cell Cycle/DNA Damage |
| HMG-CoA Reductase | Others |
| 5-HT Receptor | Proteases |
| Adrenergic Receptor | PI3K/Akt/mTOR |
| cAMP | Neuronal Signaling |
| Syk | Stem Cells & Wnt |
| Estrogen/progestogen Receptor | Angiogenesis |
| HSP90 | Others |
| p110α/γ/δ | PI3K/Akt/mTOR |
| Akt | Metabolism |
| IAP | Cell Cycle/DNA Damage |
| PI3K | PI3K/Akt/mTOR |
| GABA Receptor | Protein Tyrosine Kinase |
| PAFR | Proteases |
| Potassium Channel | Protein Tyrosine Kinase |
| Potassium Channel | Ubiquitin |
| Bcr-Abl | TGF-beta/Smad |
| PKC | Endocrinology & Hormones |
| ATPase | Ubiquitin |
| 5-HT Receptor | Cytoskeletal Signaling |
| Histone demethylases | Epigenetics |
| GPR | Metabolism |
| IGF-1R | Metabolism |
| PERK | Cytoskeletal Signaling |
| ROCK | Transmembrane Transporters |
| PLK | Ubiquitin |
| Akt | Protein Tyrosine Kinase |
| PPAR | Epigenetics |
| CSF-1R | Cell Cycle/DNA Damage |
| Liver X Receptor | Angiogenesis |
| FXR | Cell Cycle/DNA Damage |
| Trk receptor | GPCR & G Protein |
| GPR | Neuronal Signaling |
| PPAR | Apoptosis |
| PKA | Transmembrane Transporters |
| Bcl-2 | Others |
| Others | Angiogenesis |
| Epigenetic Reader Domain | Microbiology |
| BTK | Endocrinology & Hormones |
| Wnt/beta-catenin | Proteases |
| EGFR | Neuronal Signaling |
| GluR | Apoptosis |
| IκB/IKK | Others |
| MMP | Cell Cycle/DNA Damage |
| PDGFR | Neuronal Signaling |
| IκB/IKK | JAK/STAT |
| RAAS | Ubiquitin |
| PARP | Apoptosis |
| HIF | Neuronal Signaling |
| PAK | Angiogenesis |
| Topoisomerase | Cell Cycle/DNA Damage |
| Topoisomerase | Endocrinology & Hormones |
| Hydroxylase | Transmembrane Transporters |
| Kinesin | PI3K/Akt/mTOR |
| Others | Metabolism |
| CFTR | MAPK |
| FAAH | Neuronal Signaling |
| NF-κB | GPCR & G Protein |
| LPA Receptor | Transmembrane Transporters |
| LPA Receptor | NF-κB |
| CRM1 | Metabolism |
| CRM1 | Metabolism |
| CRM1 | Metabolism |
| ATM/ATR | Neuronal Signaling |
| ATM/ATR | Cytoskeletal Signaling |
| Src | Others |
| Histamine Receptor | PI3K/Akt/mTOR |
| Ftase | PI3K/Akt/mTOR |
| Hedgehog/Smoothened | Neuronal Signaling |
| ALK | Protein Tyrosine Kinase |
| BMP | Endocrinology & Hormones |
| TNF-alpha | Proteases |
| Aromatase | PI3K/Akt/mTOR |
| Dopamine Receptor | Cell Cycle/DNA Damage |
| DPP-4 | PI3K/Akt/mTOR |
| DNA Methyltransferase | Transmembrane Transporters |
| Histamine Receptor | Metabolism |
| RAAS | Angiogenesis |
| HMG-CoA Reductase | PI3K/Akt/mTOR |
| Cysteine protease | Cell Cycle/DNA Damage |
| TGF-beta/Smad | Metabolism |
| p38 MAPK | Neuronal Signaling |
| Chk | Metabolism |
| JAK | Neuronal Signaling |
| Gamma-secretase | JAK/STAT |
| CCR | Microbiology |
| Dynamin | Endocrinology & Hormones |
| AMPA Receptor-kainate | Others |
| Receptor-NMDA Receptor | |
| Adrenergic Receptor | Protein Tyrosine Kinase |
| Wee1 | Cell Cycle/DNA Damage |
| Akt | PI3K/Akt/mTOR |
| Androgen Receptor | PI3K/Akt/mTOR |
| Dehydrogenase | Protein Tyrosine Kinase |
| NOD1 | GPCR & G Protein |
| Potassium Channel | TGF-beta/Smad |
| Others | GPCR & G Protein |
| BMP | Neuronal Signaling |
| Proteasome | Proteases |
| Aurora Kinase | Endocrinology & Hormones |
| Histone Methyltransferase | Neuronal Signaling |
| p97 | Ubiquitin |
| MAO | PI3K/Akt/mTOR |
| Vasopressin Receptor | Angiogenesis |
| P2 Receptor | NF-κB |
| HER2 | Transmembrane Transporters |
| Adrenergic Receptor | TGF-beta/Smad |
| Opioid Receptor | Angiogenesis |
| COX | Cell Cycle/DNA Damage |
| Adrenergic Receptor | Cell Cycle/DNA Damage |
| TNF-alpha | JAK/STAT |
| ELF2 | Transmembrane Transporters |
| Bcr-Abl | Endocrinology & Hormones |
| Calcium Channel | DNA Damage |
| IDO | PI3K/Akt/mTOR |
| p97 | Transmembrane Transporters |
| CaSR | Metabolism |
| Rac | Metabolism |
| p53 | Cell Cycle/DNA Damage |
| MMP | Neuronal Signaling |
| E2 | Neuronal Signaling |
| DNA synthesis | Cell Cycle/DNA Damage |
| DNA-PK | Apoptosis |
| IGF-1R | Metabolism |
| Bcl2 | Apoptosis |
| GPR | JAK/STAT |
| Cathepsin K | Transmembrane Transporters |
| Histone demethylases | Metabolism |
| ATPase | Proteases |
| Cannabinoid Receptor | Neuronal Signaling |
| EGFR | Neuronal Signaling |
| IGF-1R | GPCR & G Protein |
| PDK-1 | Protein Tyrosine Kinase |
| BET | Neuronal Signaling |
| Sodium Channel | Others |
| Sodium Channel | NF-κB |
| Adrenergic Receptor | Cytoskeletal Signaling |
| Factor Xa | Metabolism |
| Others | Apoptosis |
| DUB | Neuronal Signaling |
| Caspase | Cell Cycle/DNA Damage |
| JAK | Epigenetics |
| CDK | Microbiology |
| AChR | Angiogenesis |
| HDAC | Epigenetics |
| MEK | NF-κB |
| Dopamine Receptor | Protein Tyrosine Kinase |
| MEK | Neuronal Signaling |
| c-Met | PI3K/Akt/mTOR |
| FAAH | Endocrinology & Hormones |
| S6 Kinase | GPCR & G Protein |
| Hedgehog/Smoothened | Neuronal Signaling |
| FAK | Neuronal Signaling |
| FAK | Others |
| Epigenetic Reader Domain | Microbiology |
| c-Met | Neuronal Signaling |
| CDK | Metabolism |
| Syk | NF-κB |
| p53 | Apoptosis |
| PDE | PI3K/Akt/mTOR |
| Dehydrogenase | Epigenetics |
| AChR | Angiogenesis |
| Cereblon | Others |
| Src | Others |
| DUB | Neuronal Signaling |
| DHFR | Cell Cycle/DNA Damage |
| Dopamine Receptor | PI3K/Akt/mTOR |
| Adrenergic Receptor | Apoptosis |
| Syk | Others |
| BMI | Epigenetics |
| HSP90 | Others |
| E1 Activating | Endocrinology & Hormones |
| DHFR | GPCR & G Protein |
| FLT3 | Endocrinology & Hormones |
| Integrase | Epigenetics |
| MT Receptor | Angiogenesis |
| Histamine Receptor | Others |
| MAO | Protein Tyrosine Kinase |
| Others | Protein Tyrosine Kinase |
| TGF-beta/Smad | Cytoskeletal Signaling |
| mTOR | Cell Cycle/DNA Damage |
| PLK | Cell Cycle/DNA Damage |
| Cannabinoid Receptor | Neuronal Signaling |
| Factor Xa | Neuronal Signaling |
| 5-HT Receptor | MAPK |
| ROCK | Others |
| PDE | DNA Damage |
| Dopamine Receptor | Endocrinology & Hormones |
| Histamine Receptor | Angiogenesis |
| JAK | Metabolism |
| STAT | Epigenetics |
| MAO | Metabolism |
| ELF2 | Transmembrane Transporters |
| Hedgehog/Smoothened | GPCR & G Protein |
| VEGFR | Others |
| p38 | MAPK Neuronal Signaling |
| OX Receptor | Transmembrane Transporters |
| GSK-3 | GPCR & G Protein |
| TGF-beta/Smad | Transmembrane Transporters |
| TRPV | Angiogenesis |
| 5-HT Receptor | PI3K/Akt/mTOR |
| Kinesin | Apoptosis |
| IκB/IKK | Proteases |
| Others | TGF-beta/Smad |
| MEK | Neuronal Signaling |
| Gamma-secretase | Angiogenesis |
| 5-HT Receptor | Apoptosis |
| Histone Methyltransferase | Angiogenesis |
| DNA | Methyltransferase Metabolism |
| Pim | Others |
| Sirtuin | Others |
| Endothelin Receptor | Neuronal Signaling |
| S1P Receptor | Epigenetics |
| Pim | Others |
| Topoisomerase | Cell Cycle/DNA Damage |
| CDK | Cell Cycle/DNA Damage |
| Aurora Kinase | Cell Cycle/DNA Damage |
| NF-κB | Angiogenesis |
| Raf | Transmembrane Transporters |
| PKC | Ubiquitin |
| JNK | MAPK |
| Others | Proteases |
| Sirtuin | Cell Cycle/DNA Damage |
| FGFR | Neuronal Signaling |
| STAT | Others |
| Reverse Transcriptase | Transmembrane Transporters |
| Others | Cell Cycle/DNA Damage |
| c-Met | MAPK |
| OX Receptor | Metabolism |
| PPAR | TGF-beta/Smad |
| Liver X Receptor | Endocrinology & Hormones |
| PDE | Others |
| FAK | Metabolism |
| P450 (e.g. CYP17) | Neuronal Signaling |
| GPR | Others |
| FLT3 | Endocrinology & Hormones |
| P-gp | Transmembrane Transporters |
| DUB | GPCR & G Protein |
| FLT3 | Stem Cells & Wnt |
| RAAS | Metabolism |
| mTOR | Neuronal Signaling |
| Reverse Transcriptase | Endocrinology & Hormones |
| Reverse Transcriptase | Angiogenesis |
| p53 | NF-κB |
| PI3K | Epigenetics |
| ROCK | Epigenetics |
| P2 Receptor | GPCR & G Protein |
| P2 Receptor | Neuronal Signaling |
| Tie-2 | Others |
| Carbonic Anhydrase | PI3K/Akt/mTOR |
| JAK | Angiogenesis |
| Adrenergic Receptor | MAPK |
| COX | MAPK |
| Vasopressin Receptor | Cell Cycle/DNA Damage |
| CETP | GPCR & G Protein |
| Estrogen/progestogen Receptor | Neuronal Signaling |
| Aminopeptidase | Protein Tyrosine Kinase |
| IκB/IKK | NF-kB |
| Histone demethylases | Neuronal Signaling |
| DPP-4 | Proteases |
| Sodium Channel | Others |
| HDAC | Epigenetics |
| Opioid Receptor | Others |
| AChR | Epigenetics |
| AChR | MAPK |
| GSK-3 | GPCR & G Protein |
| HER2 | Metabolism |
| Carbonic Anhydrase | NF-κB |
| MEK | Neuronal Signaling |
| Others | Epigenetics |
| MBT | GPCR & G Protein |
| FAAH | Others |
| VEGFR | Apoptosis |
| Phospholipase (e.g. PLA) | PI3K/Akt/mTOR |
| ATM/ATR | Cell Cycle/DNA Damage |
| ATM/ATR | Cell Cycle/DNA Damage |
| PARP | Apoptosis |
| Raf | Metabolism |
| DPP-4 | Metabolism |
| P450 (e.g. CYP17) | Epigenetics |
| GluR | Protein Tyrosine Kinase |
| GluR | TGF-beta/Smad |
| GluR | Cell Cycle/DNA Damage |
| Aurora Kinase | Protein Tyrosine Kinase |
| p38 MAPK | Transmembrane Transporters |
| Caspase | Endocrinology & Hormones |
| CFTR | Cell Cycle/DNA Damage |
| Wnt/beta-catenin | Metabolism |
| EGFR | Neuronal Signaling |
| AMPK | Cell Cycle/DNA Damage |
| CXCR | Angiogenesis |
| Wnt/beta-catenin | JAK/STAT |
| FXR | Neuronal Signaling |
| Survivin | MAPK |
| Gamma-secretase | Cell Cycle/DNA Damage |
| Rac | TGF-beta/Smad |
| DNA Methyltransferase | Neuronal Signaling |
| Endothelin Receptor | Neuronal Signaling |
| VEGFR | Apoptosis |
| Aurora Kinase | Stem Cells & Wnt |
| P-gp | Neuronal Signaling |

To characterize the common pathways targeted by hits on our screen, the targets of the 33 compounds were imported into ClueGo and the pathway enrichments were assessed based on GO:biological processes, KEGG, Reactome pathways and Wikipathways. Key relevant pathways included “viral carcinogensis”, histone H4 deacetylation, histone deacetylases, cell cycle, and DNA damage response (FIG. 1C, Table 3).

TABLE 3

| | | | | % Associated | Nr. | |
|---|---|---|---|---|---|---|
| | GOID | GOLevels | GOGroups | Genes | Genes | Associated Genes Found |
| Initiation of | GO:0004110 | [−1] | Group2 | 4.03 | 5.00 | [ATM, ATR, CDK2, HDAC1, |
| transcription | | | | | | HDAC2] |
| and translation | GO:0000179 | [−1] | Group2 | 10.48 | 11.00 | [ATM, ATR, CDK2, HDAC1, |
| elongation at | | | | | | HDAC2, HDAC3, HDAC4, HDAC5, |
| the HIV-1 LTR | | | | | | HDAC6, HDAC7, HDAC8] |
| | GO:0003414 | [−1] | Group2 | 21.62 | 8.00 | [HDAC1, HDAC2, HDAC3, HDAC4, |
| | | | | | | HDAC5, HDAC7, HDAC8, HDAC9] |
| histone H4 | GO:0001181 | [−1] | Group3 | 4.29 | 3.00 | [HDAC1, HDAC2, HDAC4] |
| deacetylation | GO:0003300 | [6] | Group3 | 4.17 | 3.00 | [EZH2, HDAC2, HDAC4] |
| | GO:0051153 | [5, 6, 7] | Group3 | 4.00 | 4.00 | [EZH2, HDAC4, HDAC5, HDAC9] |
| | GO:0070933 | [7, 8, 9, 10] | Group3 | 44.44 | 4.00 | [HDAC1, HDAC2, HDAC4, HDAC9] |
| Initiation of | GO:0003414 | [−1] | Group4 | 21.62 | 8.00 | [HDAC1, HDAC2, HDAC3, HDAC4, |
| transcription | | | | | | HDAC5, HDAC7, HDAC8, HDAC9] |
| and translation | GO:0000523 | [−1] | Group4 | 50.00 | 3.00 | [HDAC1, HDAC2, HDAC3] |
| elongation at | GO:0001697 | [−1] | Group4 | 5.00 | 7.00 | [EZH2, HDAC1, HDAC2, HDAC3, |
| the HIV-1 LTR | | | | | | HDAC5, HDAC7, PSMC1] |
| | GO:0002013 | [−1] | Group4 | 9.84 | 6.00 | [EZH2, HDAC1, HDAC2, HDAC3, |
| | | | | | | HDAC5, HDAC7] |
| | GO:0045814 | [5, 6, 7] | Group4 | 4.59 | 5.00 | [DNMT1, EZH2, HDAC1, HDAC2, |
| | | | | | | HDAC5] |
| histone H4 | GO:0001544 | [−1] | Group5 | 4.90 | 5.00 | [HDAC4, HDAC5, HDAC7, HDAC9, |
| deacetylation | | | | | | STAT3] |
| | GO:0002795 | [−1] | Group5 | 7.02 | 4.00 | [HDAC4, HDAC5, HDAC7, HDAC9] |
| | GO:0003414 | [−1] | Group5 | 21.62 | 8.00 | [HDAC1, HDAC2, HDAC3, HDAC4, |
| | | | | | | HDAC5, HDAC7, HDAC8, HDAC9] |
| | GO:0002005 | [−1] | Group5 | 9.38 | 3.00 | [HDAC3, HDAC4, HDAC6] |
| | GO:0003300 | [6] | Group5 | 4.17 | 3.00 | [EZH2, HDAC2, HDAC4] |
| | GO:0051153 | [5, 6, 7] | Group5 | 4.00 | 4.00 | [EZH2, HDAC4, HDAC5, HDAC9] |
| | GO:0034983 | [8, 9] | Group5 | 42.86 | 3.00 | [HDAC4, HDAC6, HDAC9] |
| | GO:0070933 | [7, 8, 9, 10] | Group5 | 44.44 | 4.00 | [HDAC1, HDAC2, HDAC4, HDAC9] |
| | GO:0090049 | [6, 7, 8, 9, | Group5 | 15.79 | 3.00 | [HDAC5, HDAC7, HDAC9] |
| | | 10, 11, 12, 13] | | | | |
| cell cycle/ | GO:0004110 | [−1] | Group6 | 4.03 | 5.00 | [ATM, ATR, CDK2, HDAC1, |
| DNA Damage | | | | | | HDAC2] |
| | GO:0004115 | [−1] | Group6 | 4.35 | 3.00 | [ATM, ATR, CDK2] |
| | GO:0000179 | [−1] | Group6 | 10.48 | 11.00 | [ATM, ATR, CDK2, HDAC1, |
| | | | | | | HDAC2, HDAC3, HDAC4, HDAC5, |
| | | | | | | HDAC6, HDAC7, HDAC8] |
| | GO:0001971 | [−1] | Group6 | 6.52 | 3.00 | [ATM, ATR, CDK2] |
| | GO:0003878 | [−1] | Group6 | 6.52 | 3.00 | [ATM, ATR, HDAC4] |
| | GO:0000707 | [−1] | Group6 | 4.35 | 3.00 | [ATM, ATR, CDK2] |
| | GO:0001757 | [−1] | Group6 | 4.55 | 3.00 | [ATM, CDK2, PSMC1] |
| | GO:0001758 | [−1] | Group6 | 4.55 | 3.00 | [ATM, CDK2, PSMC1] |
| | GO:0001762 | [−1] | Group6 | 4.41 | 3.00 | [ATM, CDK2, PSMC1] |
| HDASs deacetylate | GO:0003414 | [−1] | Group7 | 21.62 | 8.00 | [HDAC1, HDAC2, HDAC3, HDAC4, |
| histones | | | | | | HDAC5, HDAC7, HDAC8, HDAC9] |
| | GO:0000061 | [−1] | Group7 | 4.76 | 3.00 | [HDAC1, HDAC2, STAT3] |
| | GO:0000523 | [−1] | Group7 | 50.00 | 3.00 | [HDAC1, HDAC2, HDAC3] |
| | GO:0000818 | [−1] | Group7 | 5.32 | 5.00 | [HDAC1, HDAC10, HDAC2, HDAC3, |
| | | | | | | HDAC8] |
| | GO:0001181 | [−1] | Group7 | 4.29 | 3.00 | [HDAC1, HDAC2, HDAC4] |
| | GO:0001697 | [−1] | Group7 | 5.00 | 7.00 | [EZH2, HDAC1, HDAC2, HDAC3, |
| | | | | | | HDAC5, HDAC7, PSMC1] |
| | GO:0002013 | [−1] | Group7 | 9.84 | 6.00 | [EZH2, HDAC1, HDAC2, HDAC3, |
| | | | | | | HDAC5, HDAC7] |
| | GO:0003300 | [6] | Group7 | 4.17 | 3.00 | [EZH2, HDAC2, HDAC4] |
| | GO:0010870 | [4, 5, 6, 7, 8] | Group7 | 25.00 | 3.00 | [HDAC1, HDAC2, HDAC6] |
| | GO:0034983 | [8, 9] | Group7 | 42.86 | 3.00 | [HDAC4, HDAC6, HDAC9] |
| | GO:0070933 | [7, 8, 9, 10] | Group7 | 44.44 | 4.00 | [HDAC1, HDAC2, HDAC4, |
| | | | | | | HDAC9] |
| | GO:0042531 | [7, 8, 9, 10, | Group7 | 4.69 | 3.00 | [FLT3, HDAC2, STAT3] |
| | | 11, 12] | | | | |
| Viral | GO:0005034 | [−1] | Group8 | 6.11 | 11.00 | [HDAC1, HDAC10, HDAC11, HDAC2, |
| carcinomagenesis/ | | | | | | HDAC3, HDAC4, HDAC5, HDAC6, |
| Hsitone H3 | | | | | | HDAC7, HDAC8, HDAC9] |
| Deacetylation | GO:0005203 | [−1] | Group8 | 6.97 | 14.00 | [CDK2, HDAC1, HDAC10, HDAC11, |
| | | | | | | HDAC2, HDAC3, HDAC4, HDAC5, |
| | | | | | | HDAC6, HDAC7, HDAC8, HDAC9, |
| | | | | | | PSMC1, STAT3] |
| | GO:0001544 | [−1] | Group8 | 4.90 | 5.00 | [HDAC4, HDAC5, HDAC7, HDAC9, |
| | | | | | | STAT3] |
| | GO:0000179 | [−1] | Group8 | 10.48 | 11.00 | [ATM, ATR, CDK2, HDAC1, |
| | | | | | | HDAC2, HDAC3, HDAC4, HDAC5, |
| | | | | | | HDAC6, HDAC7, HDAC8] |
| | GO:0002064 | [−1] | Group8 | 10.78 | 11.00 | [HDAC1, HDAC10, HDAC11, HDAC2, |
| | | | | | | HDAC3, HDAC4, HDAC5, HDAC6, |
| | | | | | | HDAC7, HDAC8, HDAC9] |
| | GO:0002795 | [−1] | Group8 | 7.02 | 4.00 | [HDAC4, HDAC5, HDAC7, HDAC9] |
| | GO:0003414 | [−1] | Group8 | 21.62 | 8.00 | [HDAC1, HDAC2, HDAC3, HDAC4, |
| | | | | | | HDAC5, HDAC7, HDAC8, HDAC9] |
| | GO:0000221 | [−1] | Group8 | 9.91 | 11.00 | [HDAC1, HDAC10, HDAC11, HDAC2, |
| | | | | | | HDAC3, HDAC4, HDAC5, HDAC6, |
| | | | | | | HDAC7, HDAC8, HDAC9] |
| | GO:0000523 | [−1] | Group8 | 50.00 | 3.00 | [HDAC1, HDAC2, HDAC3] |
| | GO:0000562 | [−1] | Group8 | 15.07 | 11.00 | [HDAC1, HDAC10, HDAC11, HDAC2, |
| | | | | | | HDAC3, HDAC4, HDAC5, HDAC6, |
| | | | | | | HDAC7, HDAC8, HDAC9] |
| | GO:0000667 | [−1] | Group8 | 23.40 | 11.00 | [HDAC1, HDAC10, HDAC11, HDAC2, |
| | | | | | | HDAC3, HDAC4, HDAC5, HDAC6, |
| | | | | | | HDAC7, HDAC8, HDAC9] |
| | GO:0000770 | [−1] | Group8 | 18.97 | 11.00 | [HDAC1, HDAC10, HDAC11, HDAC2, |
| | | | | | | HDAC3, HDAC4, HDAC5, HDAC6, |
| | | | | | | HDAC7, HDAC8, HDAC9] |
| | GO:0000771 | [−1] | Group8 | 18.97 | 11.00 | [HDAC1, HDAC10, HDAC11, HDAC2, |
| | | | | | | HDAC3, HDAC4, HDAC5, HDAC6, |
| | | | | | | HDAC7, HDAC8, HDAC9] |
| | GO:0000773 | [−1] | Group8 | 18.97 | 11.00 | [HDAC1, HDAC10, HDAC11, HDAC2, |
| | | | | | | HDAC3, HDAC4, HDAC5, HDAC6, |
| | | | | | | HDAC7, HDAC8, HDAC9] |
| | GO:0000791 | [−1] | Group8 | 18.97 | 11.00 | [HDAC1, HDAC10, HDAC11, HDAC2, |
| | | | | | | HDAC3, HDAC4, HDAC5, HDAC6, |
| | | | | | | HDAC7, HDAC8, HDAC9] |
| | GO:0000792 | [−1] | Group8 | 18.97 | 11.00 | [HDAC1, HDAC10, HDAC11, HDAC2, |
| | | | | | | HDAC3, HDAC4, HDAC5, HDAC6, |
| | | | | | | HDAC7, HDAC8, HDAC9] |
| | GO:0000818 | [−1] | Group8 | 5.32 | 5.00 | [HDAC1, HDAC10, HDAC2, |
| | | | | | | HDAC3, HDAC8] |
| | GO:0001181 | [−1] | Group8 | 4.29 | 3.00 | [HDAC1, HDAC2, HDAC4] |
| | GO:0001697 | [−1] | Group8 | 5.00 | 7.00 | [EZH2, HDAC1, HDAC2, HDAC3, |
| | | | | | | HDAC5, HDAC7, PSMC1] |
| | GO:0002013 | [−1] | Group8 | 9.84 | 6.00 | [EZH2, HDAC1, HDAC2, HDAC3, |
| | | | | | | HDAC5, HDAC7] |
| | GO:0070932 | [7, 8, 9, 10] | Group8 | 52.38 | 11.00 | [HDAC1, HDAC10, HDAC11, HDAC2, |
| | | | | | | HDAC3, HDAC4, HDAC5, HDAC6, |
| | | | | | | HDAC7, HDAC8, HDAC9] |
| | GO:0070933 | [7, 8, 9, 10] | Group8 | 44.44 | 4.00 | [HDAC1, HDAC2, HDAC4, HDAC9] |
| | GO:0090049 | [6, 7, 8, 9, | Group8 | 15.79 | 3.00 | [HDAC5, HDAC7, HDAC9] |
| | | 10, 11, 12, 13] | | | | |

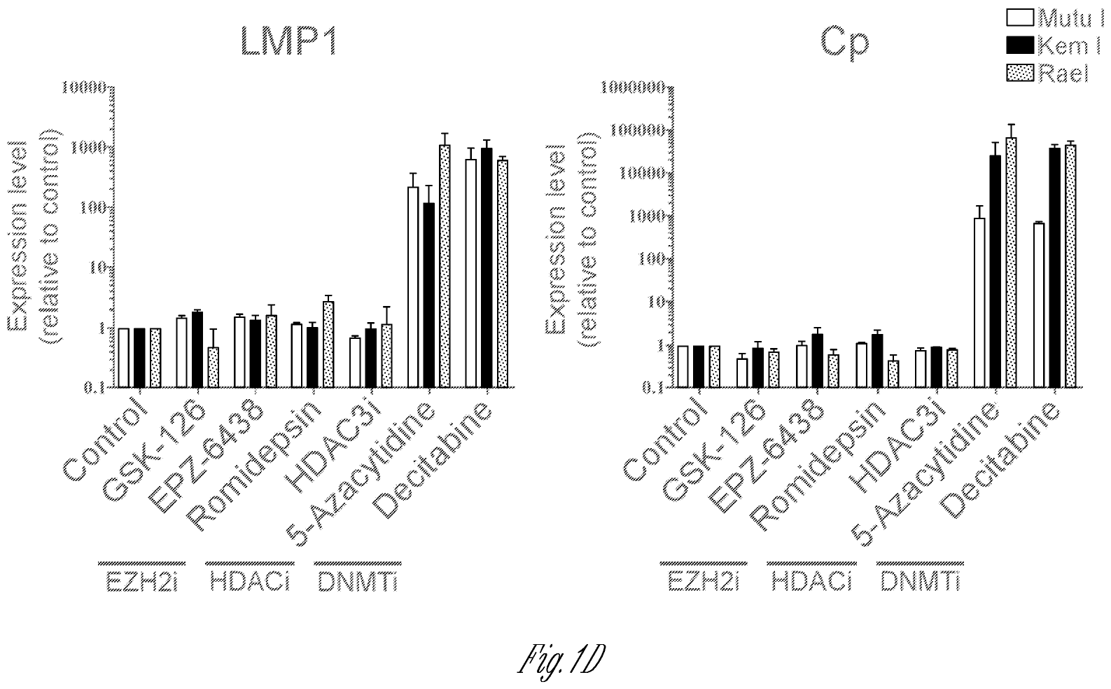

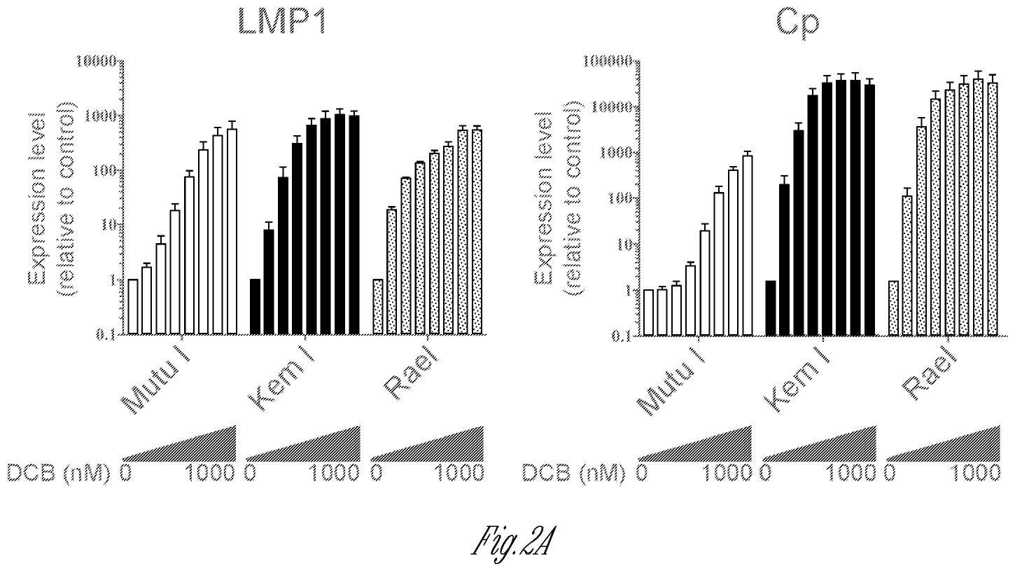

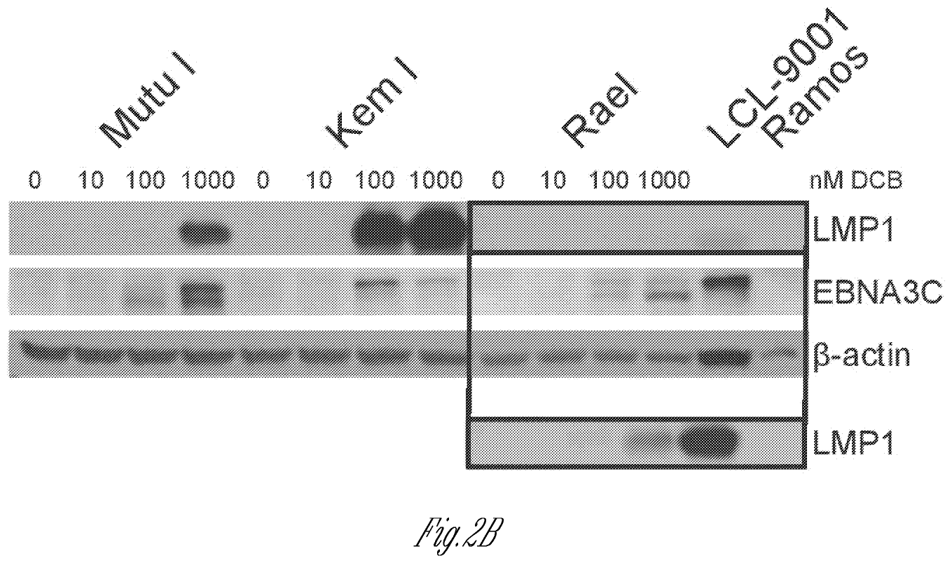

Since epigenetic modifiers were among the top hits in both the screen and pathway analyses, a focused screen of epigenetic modifying agents in Kern I, Mutu I, and Rael cell lines (FIG. 2) was performed. Cells were exposed to histone deacetylase inhibitors (HDACi), EZH2 inhibitors (EZH2i), or hypomethylating agents. To evaluate induction of latency II/III programming, qRT-PCR for LMP1 and Cp, the promoter for latency III EBNA expression, was performed. Robust induction was observed with hypomethylating agents 5-azacytidine and decitabine; which induced LMP1 and Cp>100 fold and >1000 fold respectively in all 3 cell lines.

### Decitabine Treatment Induces Expression of EBV Latency III Antigens

Since decitabine and 5-azacytadine were the top hits on the epigenetic screen, the effect of these agents on latency II/III transcript and protein expression was investigated as well as the dose-response in a panel of BL cell lines. BL cells were treated with decitabine or 5-azacytidine over a range of doses. Viral antigen expression was evaluated by qRT-PCR, immunoblot, and immunohistochemistry. After 48 hours of treatment with decitabine, dose dependent induction of LMP1 and Cp transcripts was observed in BL cells at doses as low as 25 nM (FIG. 2A). This was associated with upregulation of LMP1 and EBNA3C proteins (FIG. 2B). In contrast, with 5-azacytidine, induction of LMP1 and Cp was minimal at doses <1 μM and was not associated with significant induction of LMP1 or EBNA3C proteins (FIGS. 2A, 2B).

To determine if induction of LMP1 and EBNA3 were linked to hypomethylating agent-induced cell death, cells were exposed to decitabine or 5-azacytadine over a range of doses and cell viability evaluated with the ATP-based Cell Titer-Glo® assay. The decitabine dose that induced maximal latency II/III EBV antigen expression was 25 nM-500 nM, which was far below the IC50 of the drug, which was >5 μM (Table 4). The viability relative to untreated cells in Mutu I, Kem I and Rael cells treated with decitabine at the optimal induction dose was 62%, 128%, and 102% respectively (FIG. 8). For 5-azacytadine, the optimal dose for induction was 1-4 μM. This was closer to the IC50 of 2.2 μM->5 μM, however a similar trend is observed with minimal change in cell viability at the optimal dose for induction (FIG. 8, Table 4). This suggests that the escape from latency I in response to hypomethylating agents is not due to cell death.

TABLE 4
IC50 of hypomethylating agents in BL cells

| | 48 Hour IC50 | |
|---|---|---|
| Cell Line | 5-Azacytidine | Decitabine |
| Mutu I | 4.0 uM | >5 uM |
| Kem I | >5.0 uM | >5 uM |
| Rael | 2.2 uM | >5 uM |

Next it was determined the percentage of cells that convert to expressing latency II/II antigens after treatment with decitabine or 5-azacytadine. To do this, EBV antigen expression at the single-cell level was evaluated by immunohistochemistry (IHC) from cell blocks. Cells were treated with 5-azacytidine, decitabine, or vehicle control. Cell blocks were then evaluated by IHC for LMP1 and EBNA2. The percentage of positive cells was quantified with HALO image analysis. Decitabine treatment resulted in a significant increase in expression of EBNA2 in all three cell lines (FIG. 2E): In Mutu I, the percentage of EBNA2 increased from 0.13% to 28.3% (p=0.0004); In Kem I, 0.03% to 57.8% (p<0.0001) and in Rael, 0.24% to 37.2% (p=0.0005). The percentage of LMP1 positive cells also increased with decitabine treatment: 1.04% to 41.8% (p=0.0005), 0.31% to 54.9% (p=0.034), and 0.04% to 27.4% (p=0.048) in Mutu I, Kem I, and Rael respectively (FIG. 2F). 5-azacytidine induced a more modest expression of EBNA2 and LMP1 across the three cell lines (FIGS. 2E-F). Based on these observations, decitabine was further investigated as a potential agent to induce expression of immunogenic viral antigens in latency I tumors.

### Decitabine Induces Latency III Antigen Expression In-Vivo

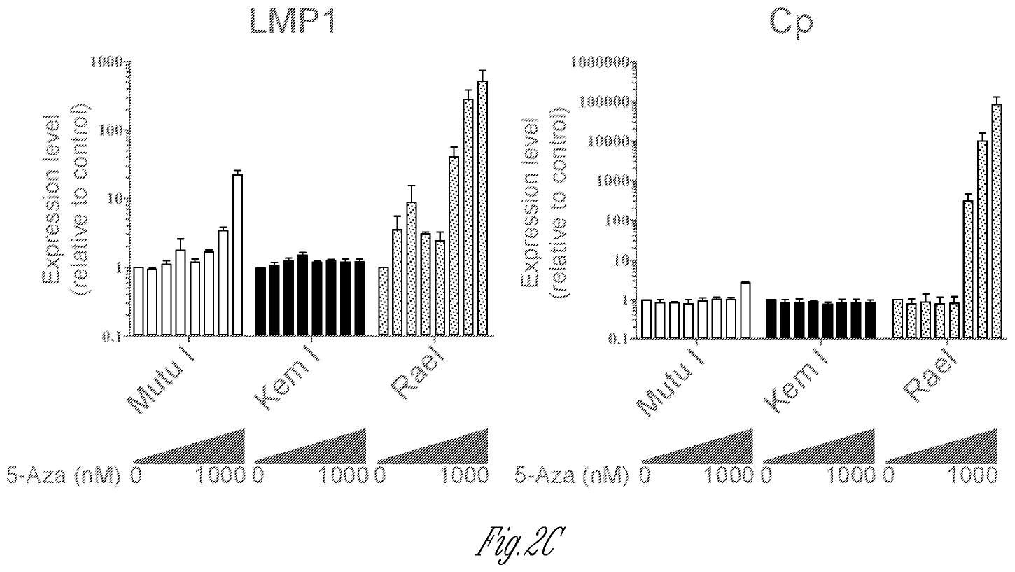

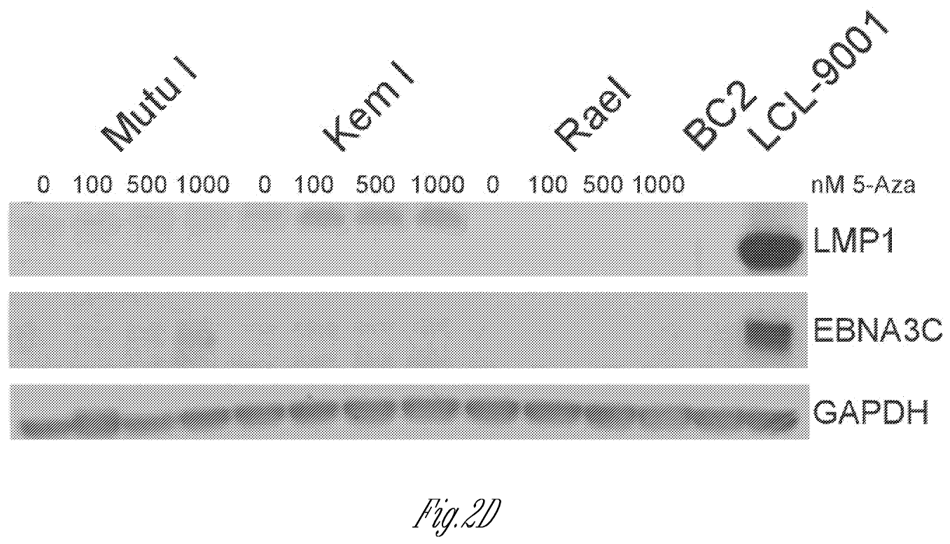

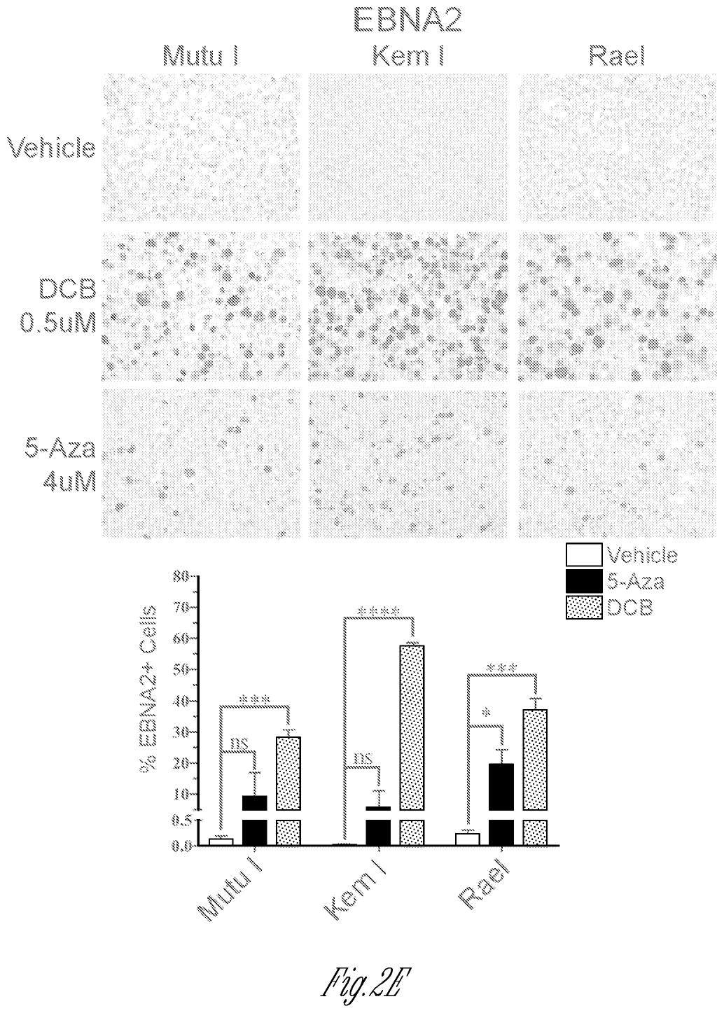

To evaluate the effect of decitabine on viral antigen expression in-vivo, we generated Mutu I, Kem I, and Rael xenografts. Upon engraftment, mice were treated with a 7-day course of decitabine (0.5 mg/kg or 1 mg/kg daily) or vehicle control. After treatment tumors were evaluated by immunohistochemistry. Vehicle treated mice had minimal or no expression of EBNA2 and LMP1 (FIGS. 3A-B). In decitabine-treated mice, EBNA2 upregulation was observed in Mutu and Rael xenografts (p=0.044 and 0.017 respectively, FIG. 3A). LMP1 expression was increased in Kem xenografts (p=0.04, FIG. 3B).

Induction of Latency III Antigens with Decitabine Persists after Removal of Drug

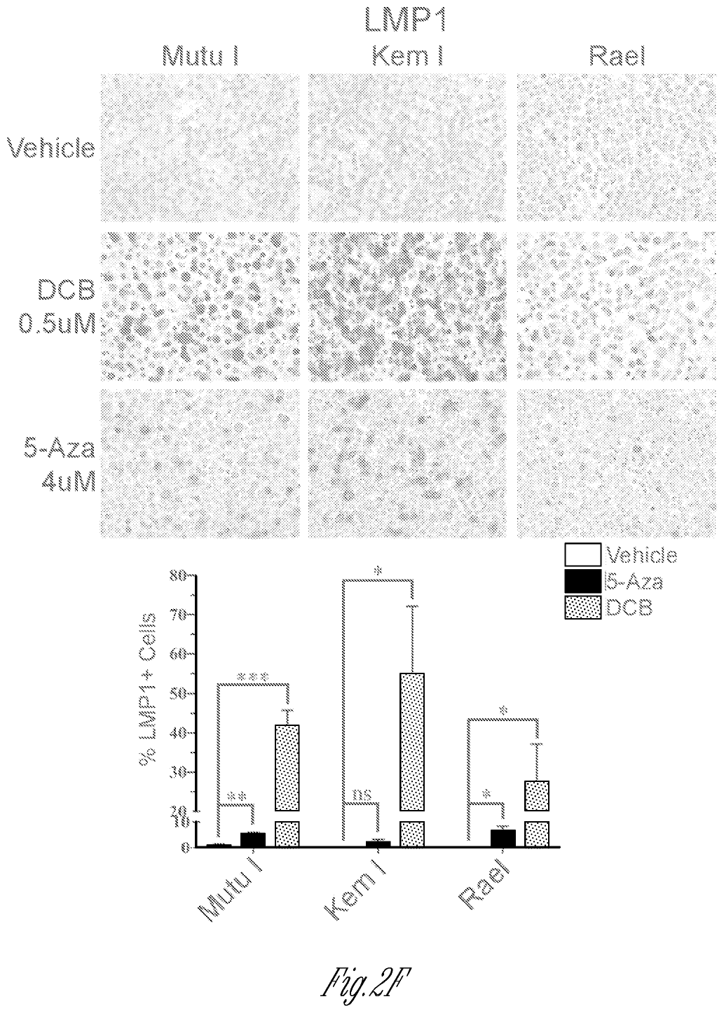

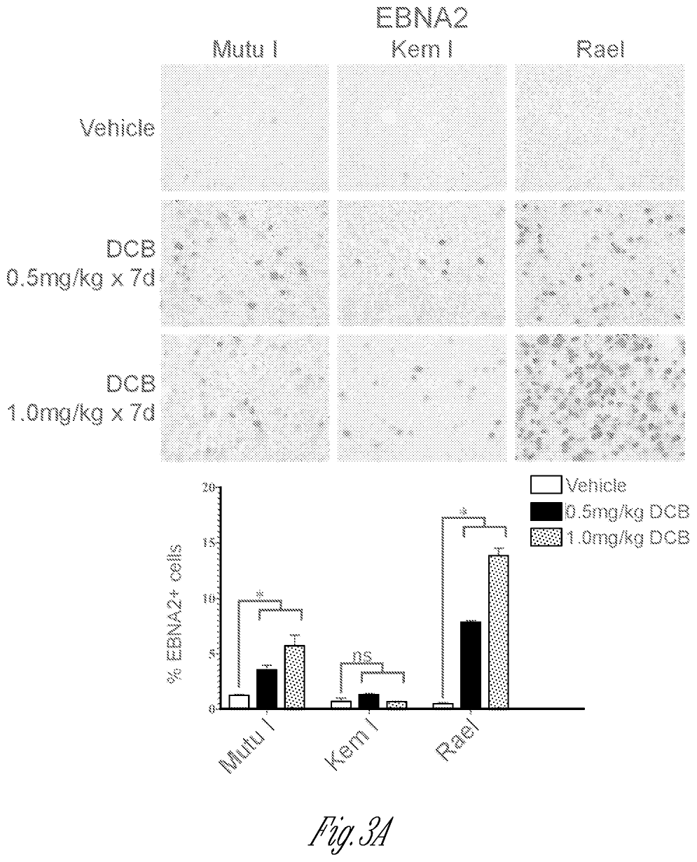

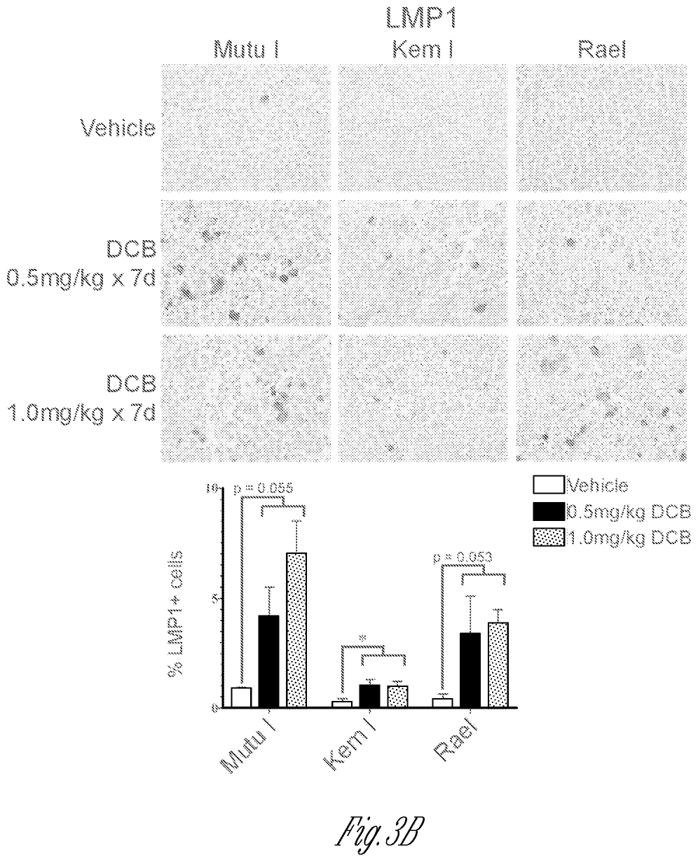

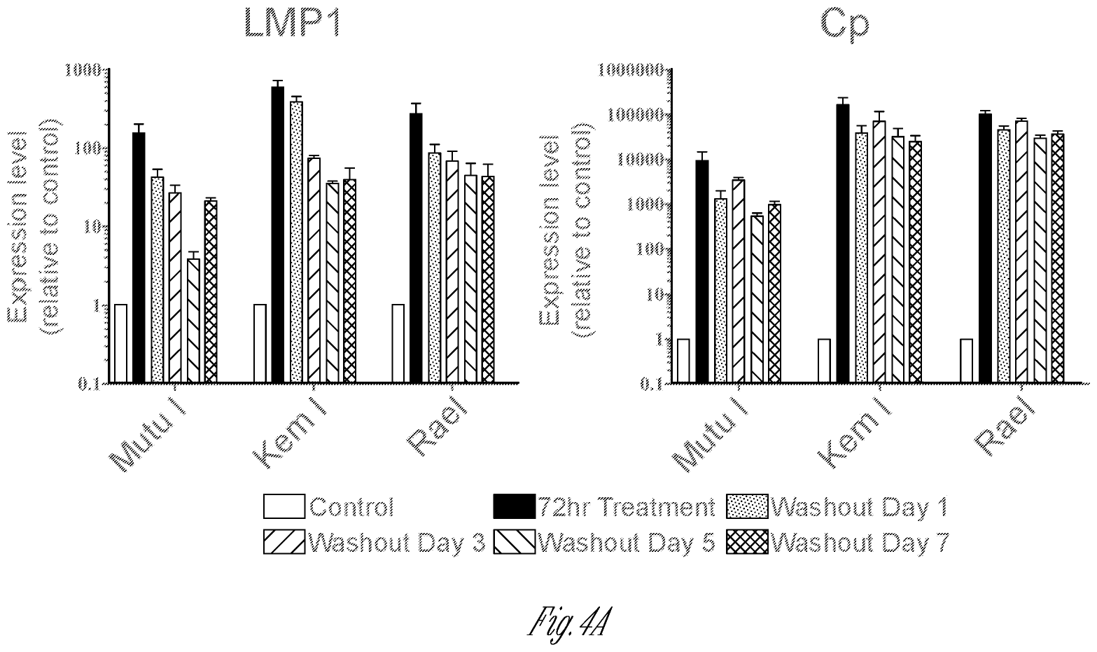

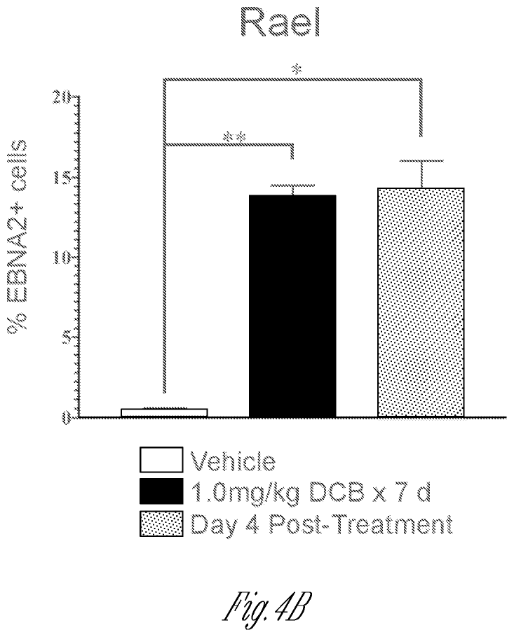

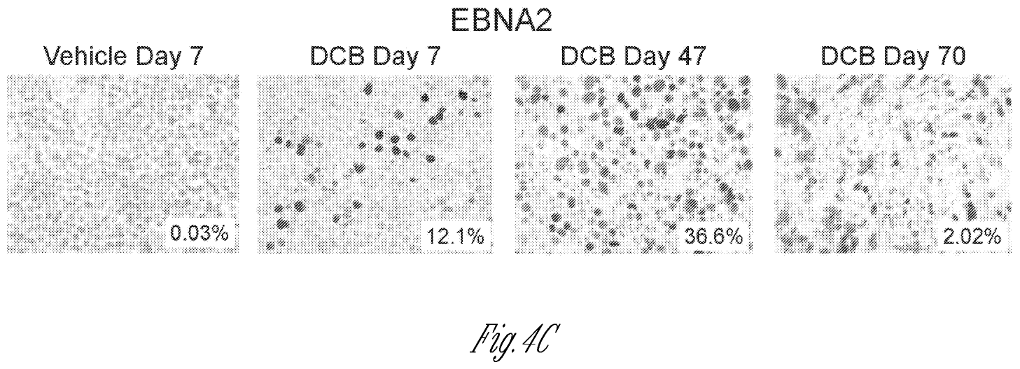

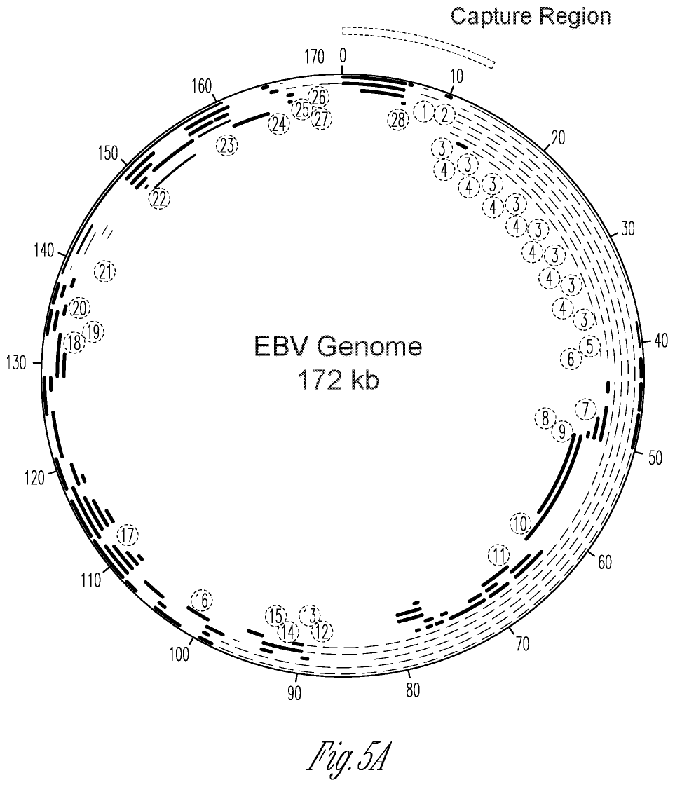

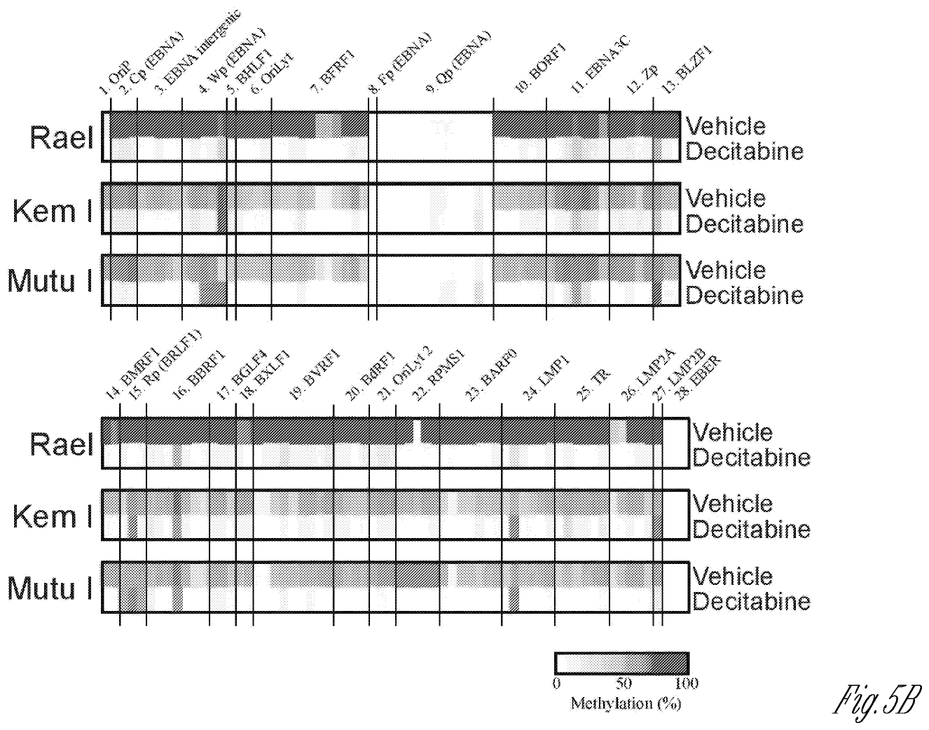

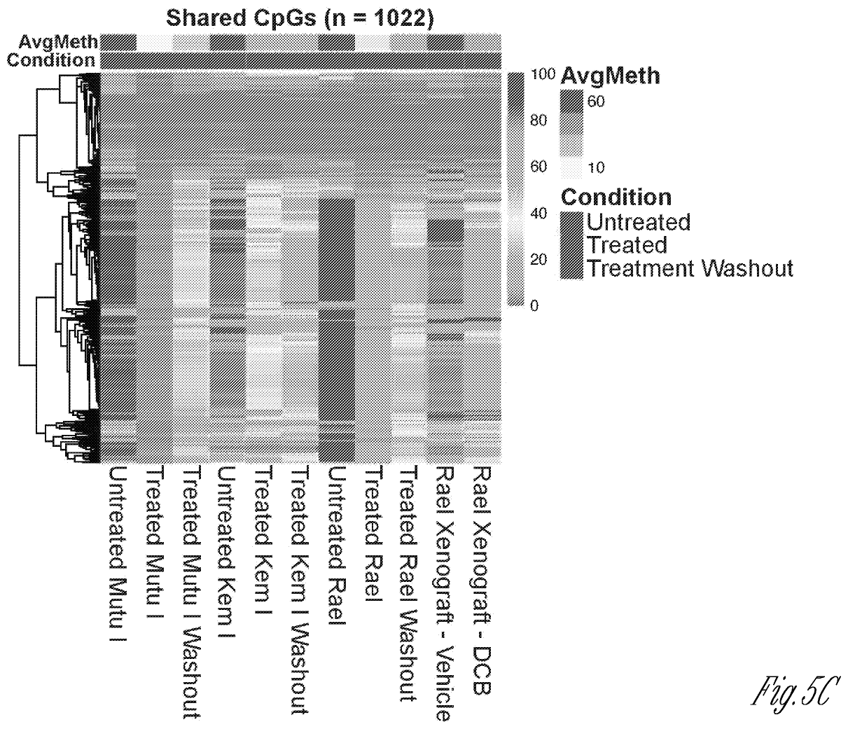

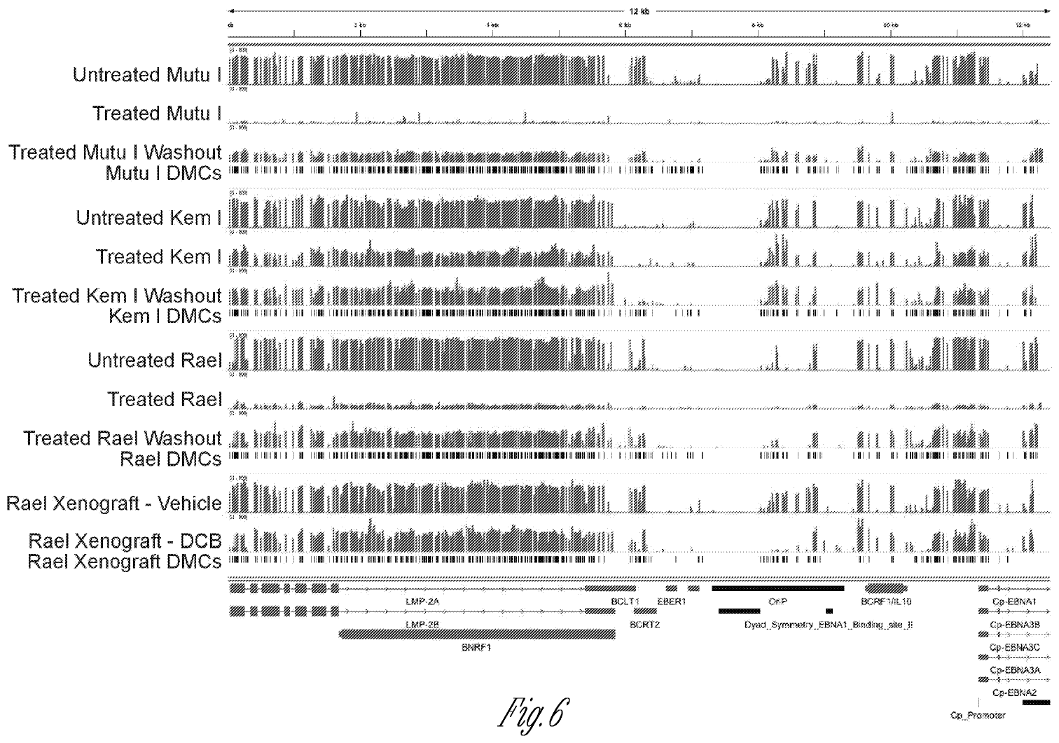

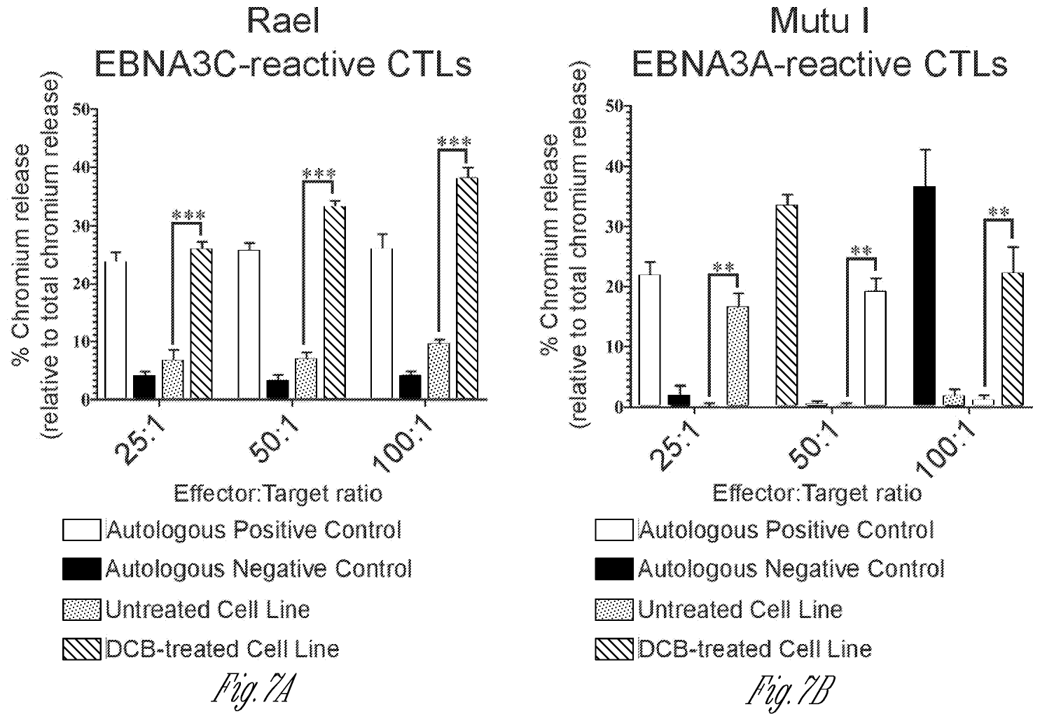

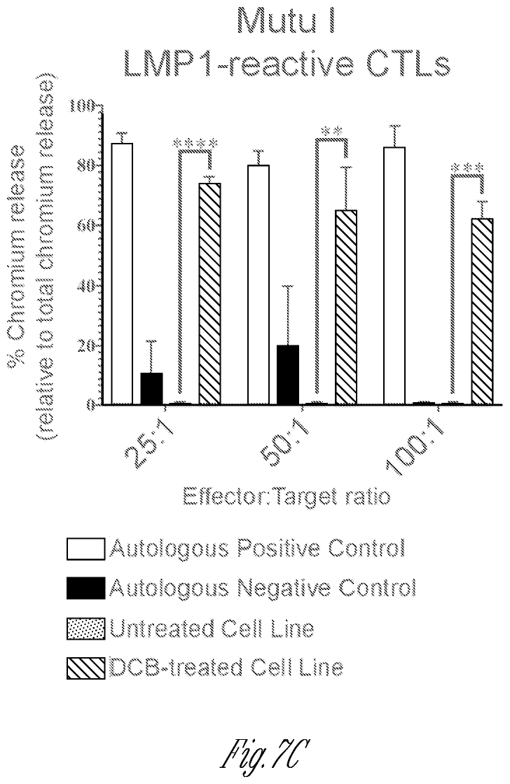

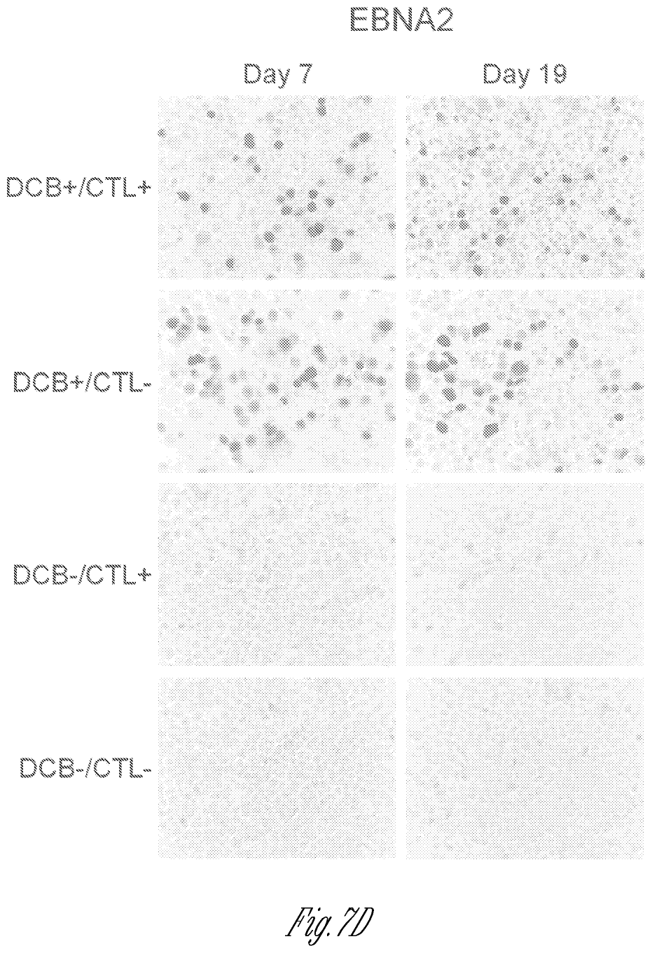

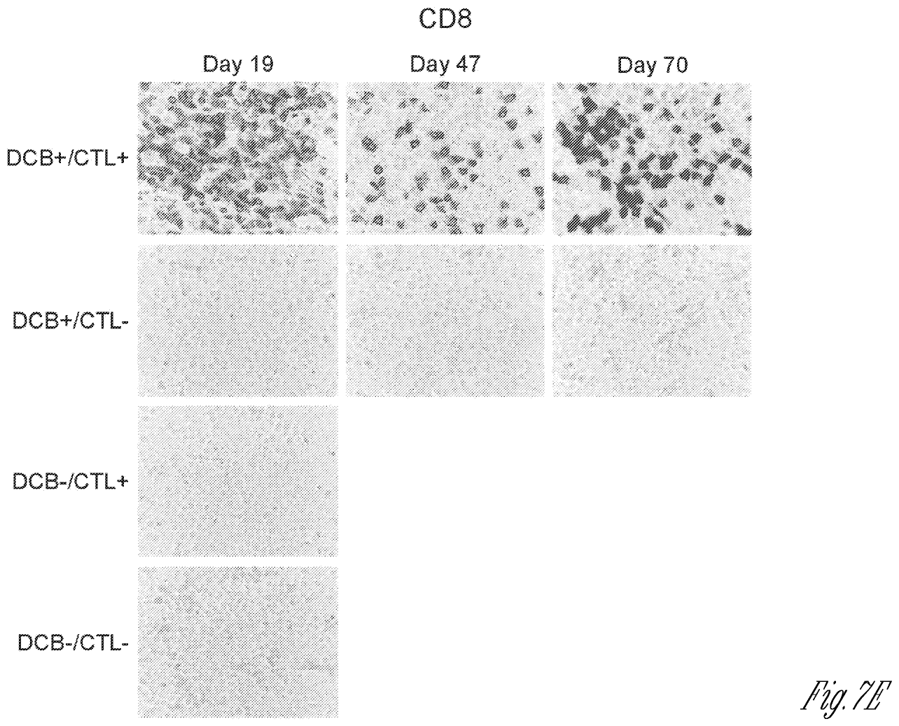

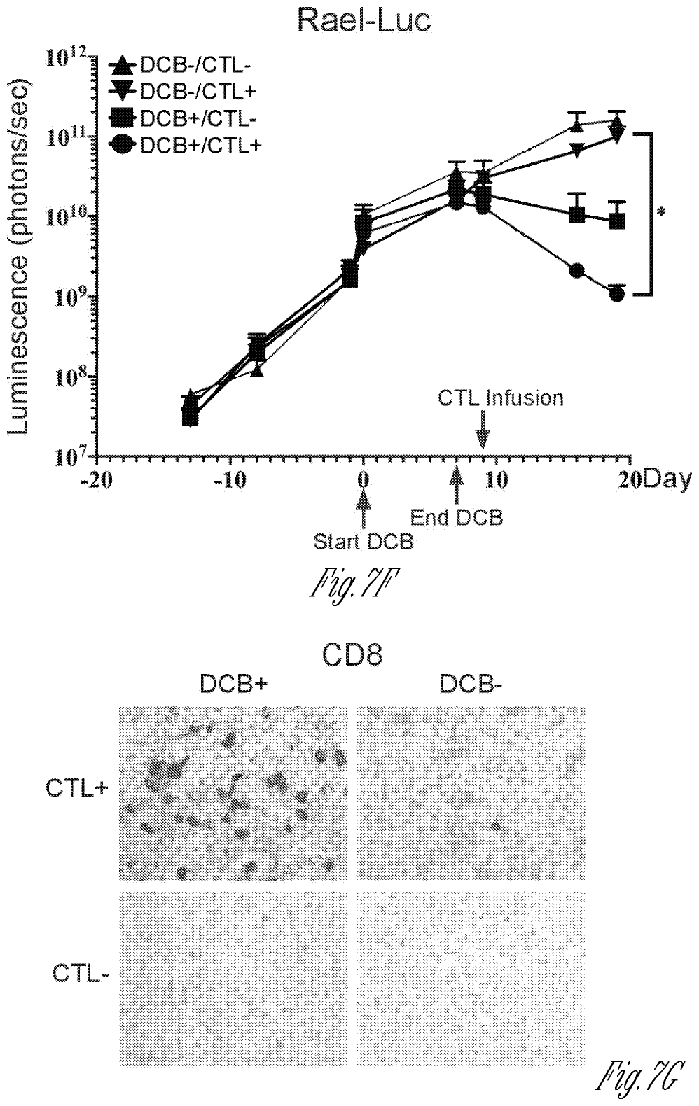

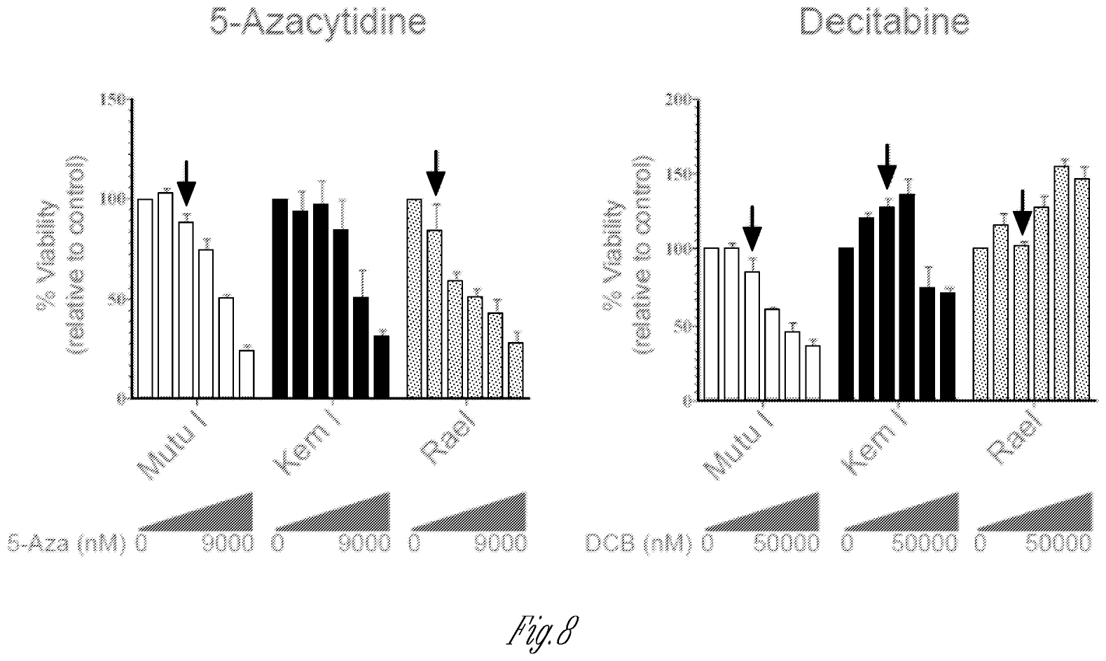

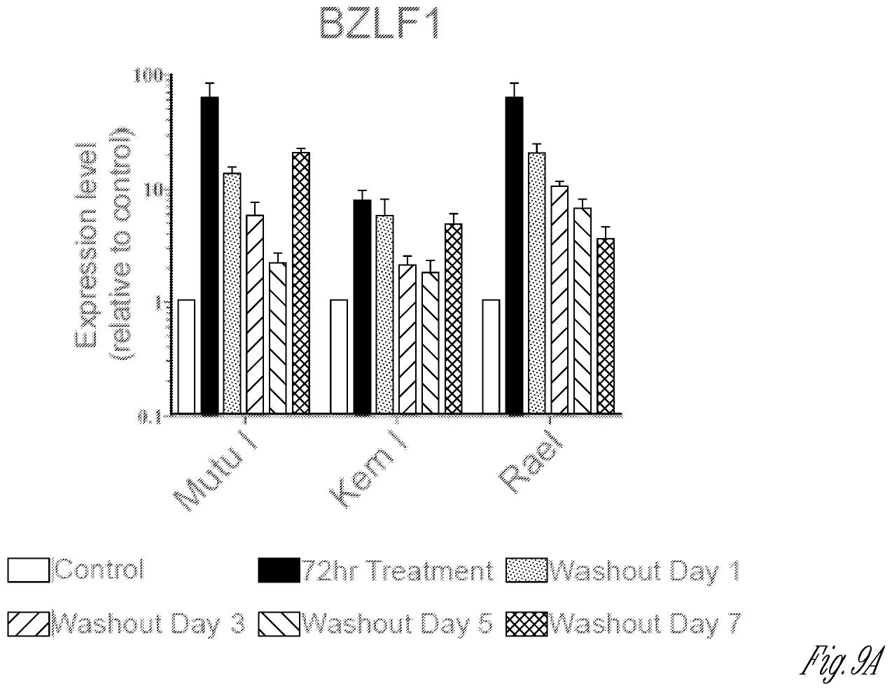

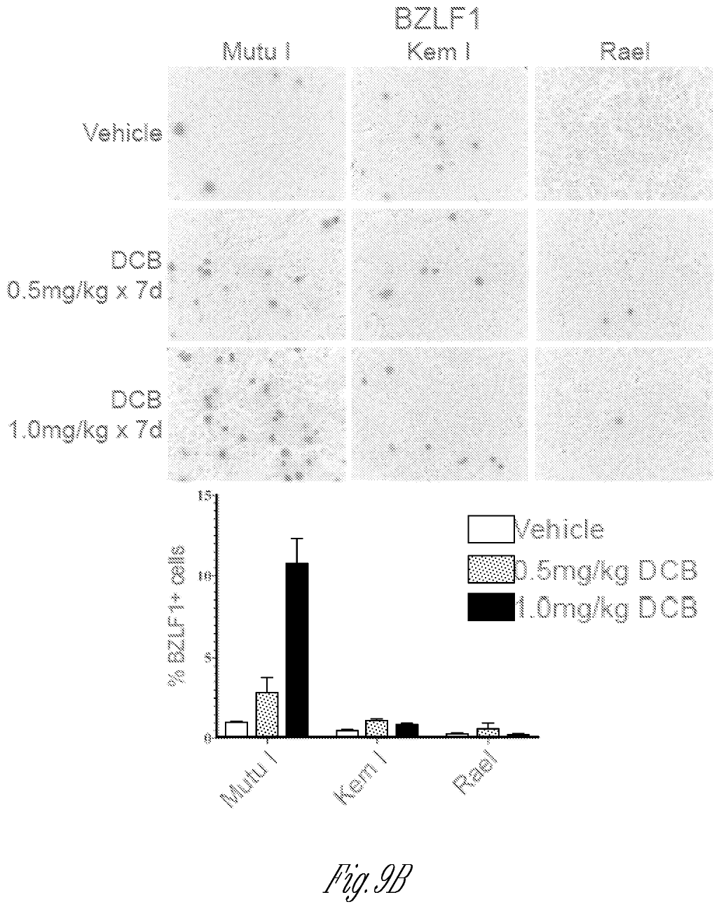

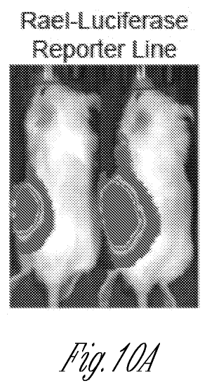

If induction of immunogenic antigens were to be used as therapeutic approach in EBV+ lymphomas, it would be important to ensure that the induction persists after removal of drug to allow time for an adequate T-cell response. The durability of latency III induction was evaluated by treating cell lines with 250 nM of decitabine for 3 days and then evaluating LMP1 and Cp promoter expression after washout of the drug. LMP1 and Cp expression by qRT-PCR persists with minimal decrement at 1, 3, 5, and 7 days after washout of decitabine (FIG. 4A). Rael xenografts were also evaluated for durability of induction in-vivo. Tumors evaluated 4 days after a 7-day course of decitabine demonstrated persistent induction with no decrement in the percentage of EBNA2 positive cells (FIG. 4B). Mice observed at later timepoints continued to express EBNA2 with some areas of tumor remaining EBNA2 positive as late as 63 days post-treatment (FIG. 4C). This suggests that epigenetic induction of latency III proteins is durable long after discontinuation of hypomethylating agents.

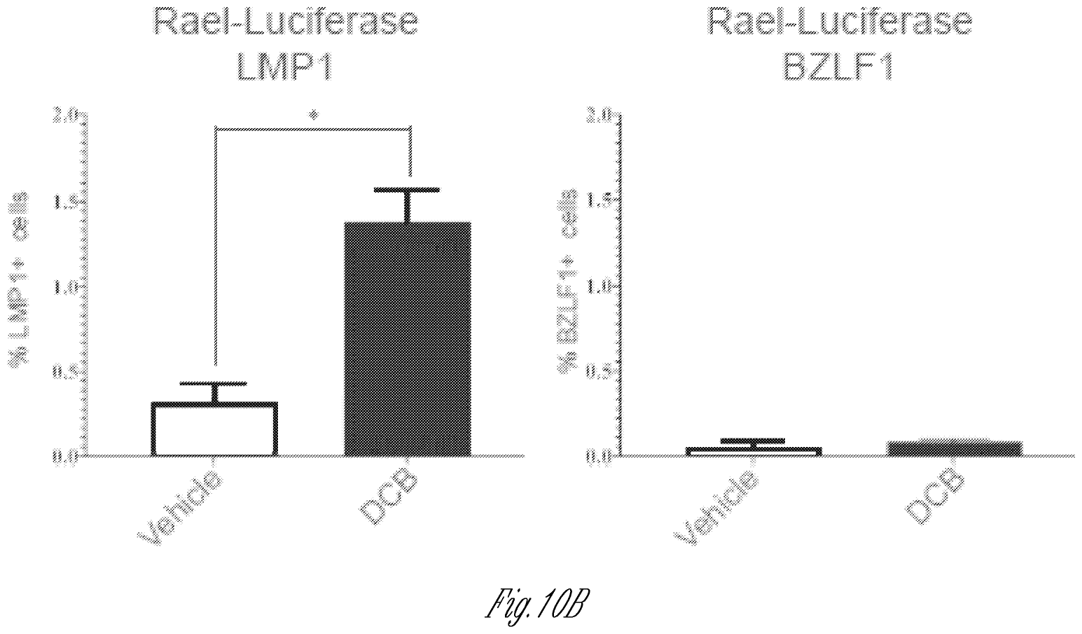

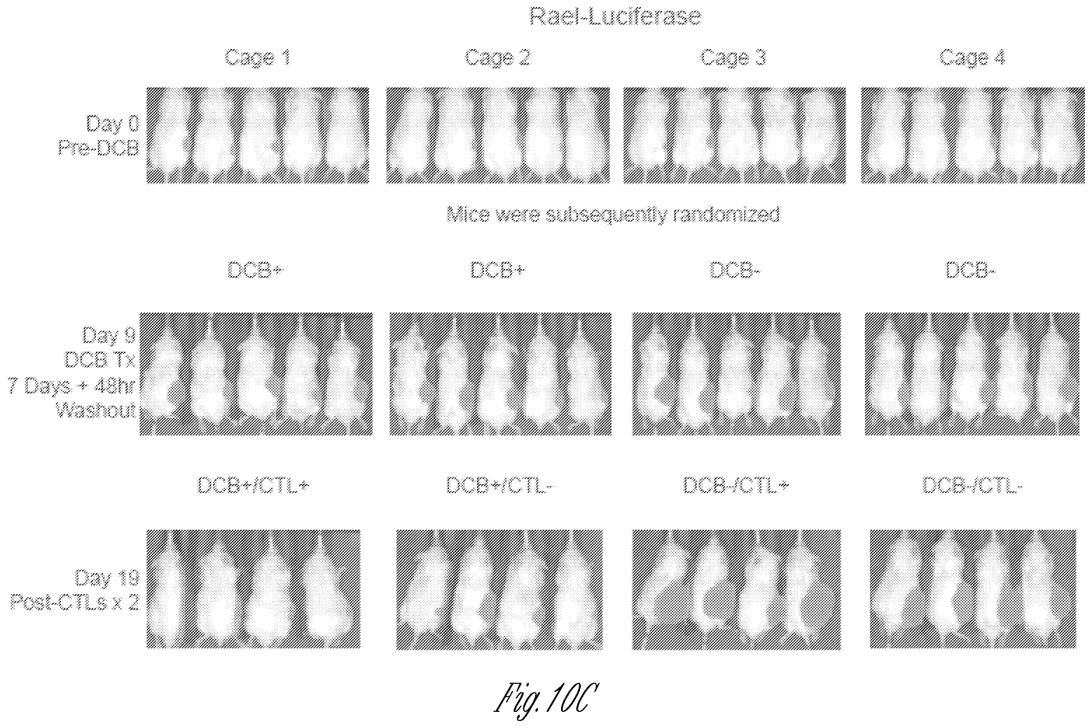

In addition to modulating latent gene expression, 5-azacytidine is known to activate lytic programming in EBV (Bhende, Seaman et al. 2004, Chan, Tao et al. 2004, Bergbauer, Kalla et al. 2010, Kalla, Gobel et al. 2012, Woellmer, Arteaga-Salas et al. 2012). Upon exposure to 5-azacytidine, the Rael cell line generates lytic and latent antigens but in distinct cell populations (Masucci, Contreras-Salazar et al. 1989). To determine if the lytic program was being activated after exposure to decitabine qRT-PCR was performed for BZLF1, the gene responsible for activating early lytic genes in Rael, Mutu I, and Kem I cells. In all three cell lines an increase in BZLF1 was observed, however this decreased over time after removal of drug (FIG. 9), suggesting that this effect may be more transient than the latent transcript activation. In the xenograft models, BLZF1 was evaluated by IHC. BLZF1 induction was observed in Mutu I but minimal or no induction was observed in Kem I or Rael, despite strong expression of EBNA2 and LMP1 in Rael (FIG. 10B). This suggests that separate lytic and latent populations are potentially being activated by decitabine, with the lytic population being more transient.

### Decitabine Induces Hypomethylation at Key Viral Promoters

The effect of decitabine on the human genome is well described, however the effect across the EBV genome is not fully characterized (Sorm, Piskala et al. 1964, Jones and Taylor 1980, Stresemann and Lyko 2008). To better understand the key regions of the EBV viral genome affected by decitabine treatment, targeted DNA methylation analyses of key viral promoters and other regions across the EBV genome were performed using MassARRAY Epityper. The assay was designed to investigate DNA methylation levels of 131 CpGs in 28 regions (1-13 CpGs per region), including EBV gene promoters, gene bodies and introns. Regions covered with this assay include Cp, LMP1, and LMP2A (FIG. 5A, Cp, LMP1, and LMP2A correspond to regions 2, 24 and 26; primers in Table 1). Kern I, Rael, and Mutu I cells were analyzed after treatment with decitabine or vehicle for 48 hours. In vehicle-treated cells, we observed a high degree of DNA methylation across the EBV genome in RaeI, and intermediate levels in Kem I and Mutu I (FIG. 5B). Following decitabine treatment, loss of methylation across the EBV viral genome was observed in all three latency I cell lines, including the Cp promoter and LMP1/2 loci, consistent with upregulation of these promoters.

To evaluate methylation with increased breadth across a focused area of the viral genome, Methyl-Capture sequencing was performed using a custom probe set designed to cover the first 13 kB of the EBV genome including the OriP, EBERs and regions upstream of Cp and EBNAs (FIG. 5A, “capture region”). Kem I, Rael, and Mutu I cells were analyzed after treatment with decitabine or vehicle as well as after decitabine followed by a 7-day washout. DNA methylation in-vivo was also assessed using tumors from Rael xenografts treated with decitabine or vehicle control.

An average of 1,046,254 reads was obtained in the untreated cells, 1,072,309 reads in the decitabine treated cells and 980,781 reads in cells treated with decitabine followed by a washout without drug (washout cells). Greater than 400,000 reads were uniquely mapped to the EBV genome in untreated, treated and washout cells. The average sequencing depth for CpGs covered in the library was 1181.

An analysis of bisulfite treated sequence reads was carried out as previously described (Garrett-Bakelman, Sheridan et al. 2015) with the modification of alignment to the EBV genome. The percentage of bisulfite converted cytosines (representing unmethylated cytosines) and non-converted cytosines (representing methylated cytosines) were recorded for each cytosine position in CpG, CHG, and CHH contexts (with H corresponding to A, C, or T nucleotides). Consistent with our MassARRAY data, global hypomethylation across the covered areas was observed after treatment with decitabine in all three cell lines (FIG. 5C). After removal of drug, a modest increase in methylation was observed, however the genome remained hypomethylated relative to treated cells (FIG. 5C). Tumors from the xenograft models displayed similar global hypomethylation after treatment with decitabine, consistent with the induction of antigens observed in these models. To evaluate the location of differentially methylated regions, differentially methylated areas were mapped to the EBV genome using Integrative Genomics Viewer (FIG. 6). Areas of differentially methylated cytosines (DMCs) included the key latency regulating region Cp as well as LMP2A/B.

### Induction of Latency III Antigens Sensitizes Tumors to T-Cell Mediated Lysis

The induction of highly immunogenic EBV antigens such as LMP1, EBNA3A and EBNA3C may sensitize tumors to autologous T-cell mediated lysis and/or killing with therapeutic administration of EBV-specific cytotoxic T-lymphocytes (EBV-CTLs). EBV-CTLs are generated in response to autologous B-cells transformed with EBV strain B95.8 and principally recognize EBNA3 or LMP1. In latency III EBV+ PTLDs which express EBNA3 and LMP1, adoptive transfer of in-vitro generated EBV-CTLs can induce durable remissions (Prockop, Doubrovina et al., Haque, Wilkie et al. 2002, Haque, Wilkie et al. 2007, Barker, Doubrovina et al. 2010).

It was hypothesized that induction of LMP1 and/or EBNA3 with decitabine treatment would sensitize resistant latency I EBV+BL tumors to third party EBV-CTLs. To identify appropriately HLA-restricted EBV-CTLs for the cell lines high resolution HLA typing was performed on Kem I, Mutu I, and Rael (Table 5). EBV-CTLs were selected from the bank of >330 GMP-grade EBV-CTL lines (Doubrovina, Oflaz-Sozmen et al. 2012). Mutu I and Rael had appropriately matched and HLA-restricted EBV-CTLs available in our biobank. This included EBV-CTLs reactive against EBAN3C, EBNA3A, and LMP1.

TABLE 5
HLA typing of BL cell lines

| | | Locus | | | | |
|---|---|---|---|---|---|---|
| | | A | B | C | DRB1 | DQB1 |
| Kem I | Allele 1 | 30:04:01G | 15:10:01 | 03:04:02 | 01:01:01G | 05:01:01 |
| | Allele 2 | 32:01:01G | 40:12 | 04:04:01 | 12:01:01G | 05:01:01 |
| Mutu I | Allele 1 | 01:01:01G | 15:03:01G | 06:02:01G | 03:01:01G | 02 (novel) |
| | Allele 2 | 02:01:01G | 45:01:01G | 08:02:01G | 07:01:01G | 02 (novel) |
| Rael | Allele 1 | 02:01:01G | 15:03:01G | 02:10:01 | 11:01:02 | 03:19:01 |
| | Allele 2 | 02:01:01G | 15:03:01G | 02:10:01 | 11:01:02 | 03:19:01 |

EBV-CTLs were tested for cytotoxicity against our latency I BL cells using a standard Cr51 release assay. EBNA3C and EBAN3A reactive T-cells were tested against Rael and Mutu I respectively (FIGS. 7A, B). A significant increase in the T-cell mediated lysis was observed in response to Rael and Mutu I cells treated with decitabine across a range of effector-to-target ratios. For example, using a 25:1 effector to target ratio, we observed 26.07% lysis with Rael cells treated with decitabine vs. 6.08% in vehicle treated cells (p=0.0026, FIG. 7A). In Mutu I cells treated with decitabine, 16.17% lysis was observed compared to 0.33% in vehicle treated cells (p=0.0018, FIG. 7B). The degree of cell lysis against decitabine treated Rael and Mutu I cells was comparable to that observed against autologous EBV-positive B-lymphoblastoid cell lines (FIG. 7A-B). To confirm these findings with a third antigen, EBV-CTLs that recognize LMP1 were evaluated. These cells were tested against Mutu I, which we found upregulated LMP1 upon exposure to decitabine (FIGS. 2D, 2F, 3B). LMP1-reactive EBV-CTLs were highly cytotoxic against decitabine-treated Mutu I but not vehicle treated Mutu I at all three effector:target ratios. For example, at a 25:1 effector to target ratio, we observed 74.11% lysis of decitabine-treated Mutu I compared to 0.67% of vehicle treated Mutu I (p<0.0001, FIG. 7C).

### Decitabine Pre-Treatment of EBV+ Tumors Results in T-Cell Homing and Inhibition of Tumor Growth In-Vivo

One potential clinical application of this work is to administer a short course of decitabine followed by appropriately HLA-restricted 3^{rd} party EBV-CTLs to patients with latency I EBV+ B-cell lymphomas. To test this approach in-vivo, EBNA3C reactive EBV-CTL responses against subcutaneous xenografts of Rael cells were evaluated in NSG mice. To quantitate responses Rael cells transduced to express luciferase (FIG. 10A) were used. Once engrafted, mice were assigned to one of four cohorts to receive decitabine vs. vehicle followed by EBV-CTLs vs. vehicle. Assignment was balanced based on BLI signal. Decitabine was administered daily for 7 days (Day 1-7) followed by two days of rest to allow drug clearance and reduce any interference between decitabine and CTLs. EBV-CTLs were then infused on day 9. Tumor burden was measured by bioluminescence. At specific timepoints, mice were humanely sacrificed to evaluate tumors for viral antigen expression and infiltration of T-cells.

Consistent with prior experiments, treatment with decitabine resulted in induction of latent antigens EBNA2 and LMP1 with minimal change in the lytic protein BZLF1 (FIG. 7D, FIG. 10B). To evaluate tumors for T-cell trafficking IHC for CD8 was performed on tumors at day 19, 47, and 70. A robust T-cell infiltrate was observed in mice that received decitabine followed by EBV-CTLs but not in mice that received vehicle followed by EBV-CTLs or any other condition (FIG. 7E). In-vivo bioluminescence imaging was performed weekly on mice to evaluate tumor burden. Inhibition of tumor growth was observed in mice who received decitabine followed by EBV-CTLs when compared to mice who received EBV-CTLs without decitabine (p=0.03, FIG. 7F and FIG. 10C). Notably, decitabine treatment did not increase PD-L1 expression (FIG. 10D) suggesting that this approach can be used without de-repressing PD-L1 in these tumors.

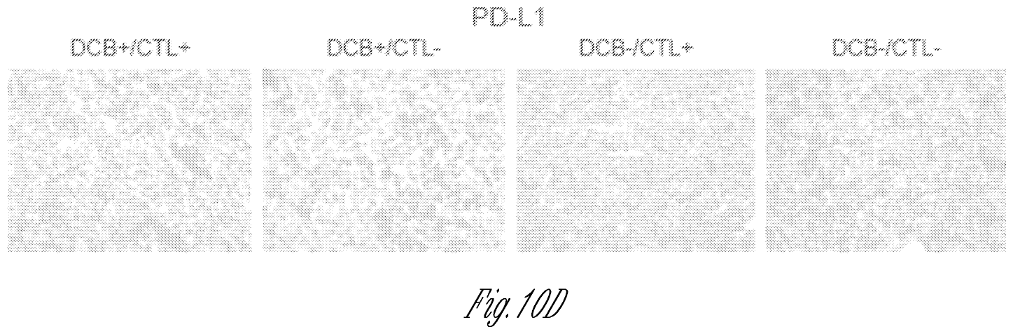

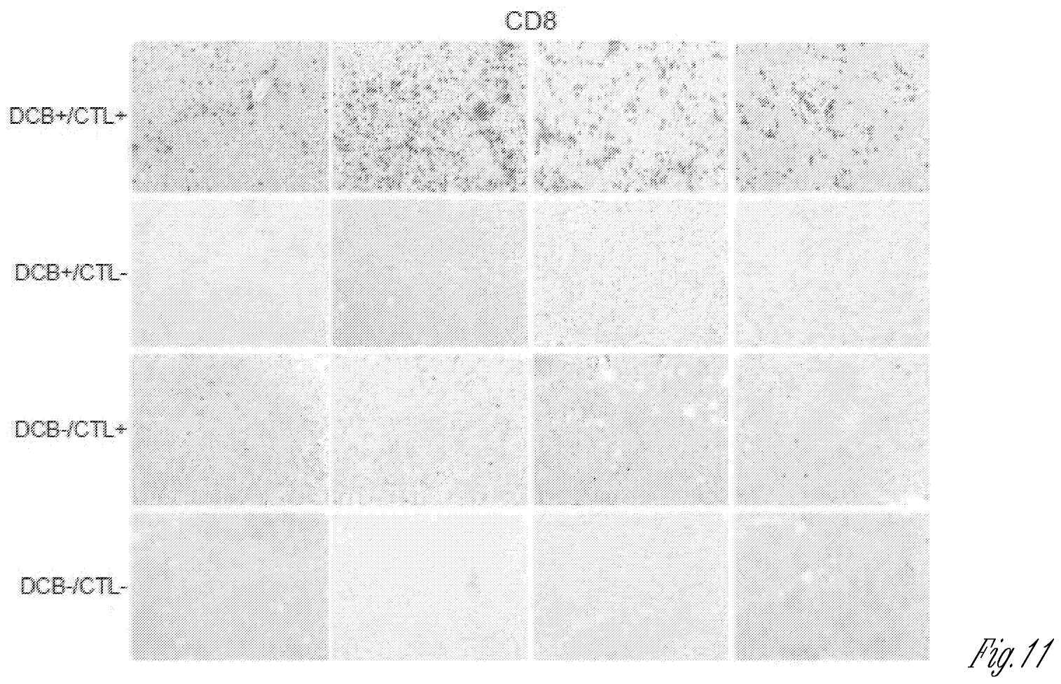

In a second xenograft model, LMP1-reactive EBV-CTLs were evaluated using Mutu I xenografts. Upon engraftment, mice were assigned to receive decitabine vs. vehicle followed by EBV-CTLs vs. vehicle as above. Mice were treated with decitabine at 1 mg/kg/day or vehicle for 3 days followed by EBT-CTLs vs. vehicle. Mutu I tumors grow rapidly in immunocompromised mice which does not allow mice to be followed over the time course needed to observe for anti-tumor effect. Rather, in this experiment, all mice were humanely sacrificed by day 18 to evaluate for T-cell homing. T-cells infiltrates were observed in the tumors of mice treated with decitabine followed by EBV-CTLs but not in the mice who received CTLs without decitabine (2.6% vs 0.08%, p=0.03; FIG. 7G, FIG. 11). These experiments demonstrate that decitabine treatment induces T-cell recognition in-vivo in latency I tumors which otherwise would not elicit a T-cell response.

### Discussion

EBV is present in nearly all cases of endemic Burkitt lymphoma in sub-Saharan Africa and approximately 30% of sporadic Burkitt lymphoma cases throughout other regions of the world (Thorley-Lawson and Allday 2008). EBV is also associated with subsets of DLBCL and classical Hodgkin lymphoma. In these tumors the virus evades immune surveillance through restricted expression of viral antigens. Therapeutic approaches that target EBV are particularly attractive in these tumors which arise in settings where high dose chemotherapy may not be feasible. One approach to EBV-directed therapy is to induce lytic viral replication and then target lytic virus with anti-herpesviral agents such as ganciclovir (Chan, Tao et al. 2004, Kenney and Mertz 2014). Attempts to sensitize tumors to ganciclovir have been limited by the strong EBV propensity to remain latent (Mentzer, Fingeroth et al. 1998, Perrine, Hermine et al. 2007, Wildeman, Novalic et al. 2012, Stoker, Novalic et al. 2015, Novalic, van Rossen et al. 2016). Our work explored a different approach: shifting latency to generate a more immunogenic tumor which could then be targeted by ex-vivo generated EBV-specific cytotoxic T-lymphocytes or, perhaps, the host immune response.

The mechanisms by which EBV maintains restricted latency are not well understood, however epigenetic modulation is likely important (Lieberman 2013, Lieberman 2016, Lu, Wiedmer et al. 2017, Wille, Li et al. 2017). The high throughput pharmacologic screen identified the hypomethylating agents 5-azacytidine and decitabine as potent inducers of LMP1 and EBNA3. No other epigenetic agents in the screen were capable of this level of induction. EBV methylation analysis performed in-vitro and in-vivo demonstrated that decitabine results in global hypomethylation across key latency promoters including LMP1 and Cp, the promoter responsible for latency III EBNA expression, suggesting that hypomethylation of these promoters can release cells from latency I. Collectively, this work demonstrates a crucial role for viral methylation in maintenance of latency in BL. Prior studies have evaluated 5-azacytidine in the Rael cell line and observed expression of Cp promoter transcripts (Masucci, Contreras-Salazar et al. 1989, Robertson, Hayward et al. 1995). Here it was show that short course, low-dose decitabine can de-repress the latency I pattern across a panel of BL cells in-vitro and in-vivo and that the effect is durable long after removal of drug, suggesting that this could be a rationale therapeutic modality to induce latency III.

A crucial unanswered question is why only a portion of EBV infected cells convert to latency III after treatment with hypomethylating agents. One possibility is that cells must be exposed to drug at a specific point in the cell cycle to allow integration of decitabine into viral DNA. Another is that some virions are inherently resistant to latency switch or activate compensatory mechanisms to maintain the restricted state. Although the induction of immunogenic antigens was observed in a subpopulation, this change rendered the cells sensitive to T-cell lysis in-vitro and resulted in substantial T-cell homing to the tumor in-vivo.

In summary, this work demonstrates that hypomethylation of EBV+BL induces expression of immunogenic viral antigens which sensitizes tumors to T-cell mediated killing. Since the induction of latency II/III antigens occurs after low dose, short course therapy with decitabine, this treatment approach followed by EBV-specific CTLs is not likely to add significant toxicity and has the potential to expand the spectrum of diseases that can be treated with third-party cytotoxic T-cells. This therapeutic approach has implications beyond EBV+ lymphomas and could potentially be applied to other EBV-driven malignancies with restricted latency.

### REFERENCES

Arvey, A., A. I. Ojesina, C. S. Pedamallu, G. Ballon, J. Jung, F. Duke, L. Leoncini, G. De Falco, E. Bressman, W. Tam, A. Chadburn, M. Meyerson and E. Cesarman (2015). “The tumor virus landscape of AIDS-related lymphomas.” Blood 125(20): e14-22.
Barker, J. N., E. Doubrovina, C. Sauter, J. J. Jaroscak, M. A. Perales, M. Doubrovin, S. E. Prockop, G. Koehne and R. J. O'Reilly (2010). “Successful treatment of EBV-associated posttransplantation lymphoma after cord blood transplantation using third-party EBV-specific cytotoxic T lymphocytes.” Blood 116(23): 5045-5049.
Bell, A. I., K. Groves, G. L. Kelly, D. Croom-Carter, E. Hui, A. T. Chan and A. B. Rickinson (2006). “Analysis of Epstein-Barr virus latent gene expression in endemic Burkitt's lymphoma and nasopharyngeal carcinoma tumour cells by using quantitative real-time PCR assays.” J Gen Virol 87(Pt 10): 2885-2890.
Bergbauer, M., M. Kalla, A. Schmeinck, C. Gobel, U. Rothbauer, S. Eck, A. Benet-Pages, T. M. Strom and W. Hammerschmidt (2010). “CpG-methylation regulates a class of Epstein-Barr virus promoters.” PLoS Pathog 6(9): e1001114.
Bhende, P. M., W. T. Seaman, H. J. Delecluse and S. C. Kenney (2004). “The EBV lytic switch protein, Z, preferentially binds to and activates the methylated viral genome.” Nat Genet 36(10): 1099-1104.
Bollard, C. M., S. Gottschalk, V. Torrano, O. Diouf, S. Ku, Y. Hazrat, G. Carrum, C. Ramos, L. Fayad, E. J. Shpall, B. Pro, H. Liu, M. F. Wu, D. Lee, A. M. Sheehan, Y. Zu, A. P. Gee, M. K. Brenner, H. E. Heslop and C. M. Rooney (2014). “Sustained complete responses in patients with lymphoma receiving autologous cytotoxic T lymphocytes targeting Epstein-Barr virus latent membrane proteins.” J Clin Oncol 32(8): 798-808.
Burkitt, D. (1958). “A sarcoma involving the jaws in African children.” Br J Surg 46(197): 218-223.
Carbone, A., A. Gloghini and G. Dotti (2008). “EBV-associated lymphoproliferative disorders: classification and treatment.” Oncologist 13(5): 577-585.
Cesarman, E. (2014). “How do viruses trick B cells into becoming lymphomas?” Curr Opin Hematol 21(4): 358-368.
Chan, A. T., Q. Tao, K. D. Robertson, I. W. Flinn, R. B. Mann, B. Klencke, W. H. Kwan, T. W. Leung, P. J. Johnson and R. F. Ambinder (2004). “Azacitidine induces demethylation of the Epstein-Barr virus genome in tumors.” J Clin Oncol 22(8): 1373-1381.
Cohen J I, Fauci A S, Varmus H, Nabel G J. Epstein-Barr virus: an important vaccine target for cancer prevention. Sci Transl Med. 2011; 3(107):107.
Dierickx, D., T. Tousseyn and O. Gheysens (2015). “How I treat posttransplant lymphoproliferative disorders.” Blood 126(20): 2274-2283.
Doubrovina, E., B. Oflaz-Sozmen, S. E. Prockop, N. A. Kernan, S. Abramson, J. Teruya-Feldstein, C. Hedvat, J. F. Chou, G. Heller, J. N. Barker, F. Boulad, H. Castro-Malaspina, D. George, A. Jakubowski, G. Koehne, E. B. Papadopoulos, A. Scaradavou, T. N. Small, R. Khalaf, J. W. Young and R. J. O'Reilly (2012). “Adoptive immunotherapy with unselected or EBV-specific T cells for biopsy-proven EBV+ lymphomas after allogeneic hematopoietic cell transplantation.” Blood 119(11): 2644-2656.
Garrett-Bakelman, F. E., C. K. Sheridan, T. J. Kacmarczyk, J. Ishii, D. Betel, A. Alonso, C. E. Mason, M. E. Figueroa and A. M. Melnick (2015). “Enhanced reduced representation bisulfite sequencing for assessment of DNA methylation at base pair resolution.” J Vis Exp (96): e52246.
Haque, T., G. M. Wilkie, M. M. Jones, C. D. Higgins, G. Urquhart, P. Wingate, D. Burns, K. McAulay, M. Turner, C. Bellamy, P. L. Amlot, D. Kelly, A. MacGilchrist, M. K. Gandhi, A. J. Swerdlow and D. H. Crawford (2007). “Allogeneic cytotoxic T-cell therapy for EBV-positive posttransplantation lymphoproliferative disease: results of a phase 2 multicenter clinical trial.” Blood 110(4): 1123-1131.
Haque, T., G. M. Wilkie, C. Taylor, P. L. Amlot, P. Murad, A. Iley, D. Dombagoda, K. M. Britton, A. J. Swerdlow and D. H. Crawford (2002). “Treatment of Epstein-Barr-virus-positive post-transplantation lymphoproliferative disease with partly HLA-matched allogeneic cytotoxic T cells.” Lancet 360(9331): 436-442.
Jones, P. A. and S. M. Taylor (1980). “Cellular differentiation, cytidine analogs and DNA methylation.” Cell 20(1): 85-93.
Kalla, M., C. Gobel and W. Hammerschmidt (2012). “The lytic phase of epstein-barr virus requires a viral genome with 5-methylcytosine residues in CpG sites.” J Virol 86(1): 447-458.
Kenney, S. C. and J. E. Mertz (2014). “Regulation of the latent-lytic switch in Epstein-Barr virus.” Semin Cancer Biol 26: 60-68.
LaCasce, A. S. (2006). “Post-transplant lymphoproliferative disorders.” Oncologist 11(6): 674-680.
Lieberman, P. M. (2013). “Keeping it quiet: chromatin control of gammaherpesvirus latency.” Nat Rev Microbiol 11(12): 863-875.
Lieberman, P. M. (2016). “Epigenetics and Genetics of Viral Latency.” Cell Host Microbe 19(5): 619-628.
Lu, F., A. Wiedmer, K. A. Martin, P. Wickramasinghe, A. V. Kossenkov and P. M. Lieberman (2017). “Coordinate Regulation of TET2 and EBNA2 Controls the DNA Methylation State of Latent Epstein-Barr Virus.” J Virol 91(20).
Masucci, M. G., B. Contreras-Salazar, E. Ragnar, K. Falk, J. Minarovits, I. Ernberg and G. Klein (1989). “5-Azacytidine up regulates the expression of Epstein-Barr virus nuclear antigen 2 (EBNA-2) through EBNA-6 and latent membrane protein in the Burkitt's lymphoma line rael.” J Virol 63(7): 3135-3141.
McLaughlin-Drubin Me., Munger K. Viruses associated with human cancer. Biochim Biophys Acta. 2008; 1782(3):127-150.
Mentzer, S. J., J. Fingeroth, J. J. Reilly, S. P. Perrine and D. V. Faller (1998). “Arginine butyrate-induced susceptibility to ganciclovir in an Epstein-Barr-virus-associated lymphoma.” Blood Cells Mol Dis 24(2): 114-123.
Novalic, Z., T. van Rossen, A. E. Greijer and J. M. Middeldorp (2016). “Agents and Approaches for Lytic Induction Therapy of Esptein-Barr Virus Associated Malignancies.” Med Chem (Los Angeles) 6: 449-466.
Perrine, S. P., O. Hermine, T. Small, F. Suarez, R. O'Reilly, F. Boulad, J. Fingeroth, M. Askin, A. Levy, S. J. Mentzer, M. Di Nicola, A. M. Gianni, C. Klein, S. Horwitz and D. V. Faller (2007). “A phase 1/2 trial of arginine butyrate and ganciclovir in patients with Epstein-Barr virus-associated lymphoid malignancies.” Blood 109(6): 2571-2578.
Prockop, S. E., E. S. Doubrovina, J. N. Barker, K. Baroudy, F. Boulad, N. A. Kernan, R. Khalaf, R. Kobos, E. Papadopoulos, C. Sauter, A. Scaradavou, S. Suser, G. Wasilewski, N. Zakak and R. O'Reilly “Third Party Donor Derived EBV Specific T Cells for the Treatment of Refractory EBV-Related Post-Transplant Lymphomas.” Biology of Blood and Marrow Transplantation 20(2): S49-S50.
Robertson, K. D., S. D. Hayward, P. D. Ling, D. Samid and R. F. Ambinder (1995). “Transcriptional activation of the Epstein-Barr virus latency C promoter after 5-azacytidine treatment: evidence that demethylation at a single CpG site is crucial.” Mol Cell Biol 15(11): 6150-6159.
Roskrow, M. A., N. Suzuki, Y. Gan, J. W. Sixbey, C. Y. Ng, S. Kimbrough, M. Hudson, M. K. Brenner, H. E. Heslop and C. M. Rooney (1998). “Epstein-Barr virus (EBV)-specific cytotoxic T lymphocytes for the treatment of patients with EBV-positive relapsed Hodgkin's disease.” Blood 91(8): 2925-2934.
Sorm, F., A. Piskala, A. Cihak and J. Vesely (1964). “5-Azacytidine, a new, highly effective cancerostatic.” Experientia 20(4): 202-203.
Stoker, S. D., Z. Novalic, M. A. Wildeman, A. D. Huitema, S. A. Verkuijlen, H. Juwana, A. E. Greijer, I. B. Tan, J. M. Middeldorp and J. P. de Boer (2015). “Epstein-Barr virus-targeted therapy in nasopharyngeal carcinoma.” J Cancer Res Clin Oncol 141(10): 1845-1857.
Stresemann, C. and F. Lyko (2008). “Modes of action of the DNA methyltransferase inhibitors azacytidine and decitabine.” Int J Cancer 123(1): 8-13.
Thorley-Lawson, D. A. and M. J. Allday (2008). “The curious case of the tumour virus: 50 years of Burkitt's lymphoma.” Nat Rev Microbiol 6(12): 913-924.
Wildeman, M. A., Z. Novalic, S. A. Verkuijlen, H. Juwana, A. D. Huitema, I. B. Tan, J. M. Middeldorp, J. P. de Boer and A. E. Greijer (2012). “Cytolytic virus activation therapy for Epstein-Barr virus-driven tumors.” Clin Cancer Res 18(18): 5061-5070.
Wille, C. K., Y. Li, L. Rui, E. C. Johannsen and S. C. Kenney (2017). “Restricted TET2 Expression in Germinal Center Type B Cells Promotes Stringent Epstein-Barr Virus Latency.” J Virol 91(5).
Woellmer, A., J. M. Arteaga-Salas and W. Hammerschmidt (2012). “BZLF1 governs CpG-methylated chromatin of Epstein-Barr Virus reversing epigenetic repression.” PLoS Pathog 8(9): e1002902.

All publications, patents and patent applications are incorporated herein by reference. While in the foregoing specification, this invention has been described in relation to certain embodiments thereof, and many details have been set forth for purposes of illustration, it will be apparent to those skilled in the art that the invention is susceptible to additional embodiments and that certain of the details herein may be varied considerably without departing from the basic principles of the invention.

1. A method to convert EBV latency I tumors in a mammal to EBV latency II/III tumors or to sensitize EBV+ tumors in a mammal to T-cell mediated killing, comprising: administering to the mammal a composition comprising an effective amount of one or more hypomethylating agents or DNA methyl transferase inhibitors.

2. (canceled)

3. A method to modulate viral immunogenicity in a mammal having EBV+ lymphoma, comprising: administering to the mammal a composition comprising an effective amount of one or more hypomethylating agents or DNA methyl transferase inhibitors.

4. The method of claim 1 wherein the mammal is a human.

5. The method of claim 1 wherein the mammal has Burkitt's lymphoma or diffuse large B-cell lymphoma (DLBCL).

6. (canceled)

7. The method of claim 1 wherein the mammal has Hodgkin lymphoma.

8. The method of claim 1 wherein the mammal has nasopharyngeal cancer or gastric cancer.

9. The method of claim 1 wherein the agent increases expression of LMP1, EBNA3C, or both.

10. The method of claim 1 wherein the hypomethylating agent comprises decitabine or azacytidine.

11. The method of claim 1 wherein the agent is a methyltransferase inhibitor.

12. The method of claim 1 wherein the hypomethylating agent is systemically administered.

13. The method of claim 1 wherein the hypomethylating agent is orally administered.

14. The method of claim 1 wherein the hypomethylating agent is injected.

15. The method of claim 1 further comprising administering an immunotherapeutic.

16. The method of claim 15 wherein the immunotherapeutic comprises EBV-specific cytotoxic T-cells.

17. The method of claim 15 wherein the immunotherapeutic is a checkpoint inhibitor.

18. The method of claim 15 wherein the immunotherapeutic is injected.

19. The method of claim 15 wherein the immunotherapeutic is systemically administered.

20. The method of 15 wherein the immunotherapeutic is orally administered.

21. An in vitro method to detect an agent that converts EBV latency I tumor cells to EBV latency II/III tumors, comprising:
contacting EBV latency I tumor cells with one or more agents; and
determining whether the one or more agents convert the EBV latency I tumor cells to EBV latency II/II tumor cells.

22. (canceled)

23. The method of claim 21 wherein the agent increases expression of LMP1, EBNA3C, or both, or wherein the agent increases expression of BLZF1.

24-26. (canceled)
